(19) United States
(12) Patent Application Publication
(10) Pub. No.: US 2009/0304738 A1
(43) Pub. Date: Dec. 10, 2009

(54) Methods for Enhancing Immune Responses

(76) Inventors: Thomas M. Moran, New York, NY (US); Carolina B. Lopez, New York, NY (US); Jacob Yount, Brooklyn, NY (US)

Correspondence Address: KLAUBER & JACKSON, 411 HACKENSACK AVENUE, HACKENSACK, NJ, 07601, US

(21) Appl. No.: 11/922,427

(22) PCT Filed: Jun. 15, 2006
(86) PCT No.: PCT/US2006/023267

§ 371 Date: Aug. 13, 2009

Related U.S. Application Data
(60) Provisional application No. 60/691,312, filed on Jun. 16, 2005.

(51) Int. Cl. A61K 39/12; C12N 7/01; C12N 5/02

(52) U.S. Cl. 424/205.1; 424/204.1; 435/235.1; 435/375

(57) ABSTRACT

The present invention is directed to methods for enhancing immune responses. Such methods serve to enhance dendritic cell activation, which, in turn, promotes a more robust immune response to foreign antigens. As such, the methods and compositions of the invention are for useful in the context of a variety of prophylactic and therapeutic regimens.

### CROSS REFERENCE TO RELATED APPLICATION

This application claims priority under 35 USC §119(e) from U.S. Provisional Application Ser. No. 60/691,312, filed Jun. 16, 2005, which application is herein specifically incorporated by reference in its entirety.

### FIELD OF THE INVENTION

The present invention relates to the fields of molecular biology and immunology. The invention is principally directed to methods for enhancing immune responses. Such methods serve to enhance dendritic cell activation, which, in turn, promotes a more robust immune response to foreign antigens. In a particular aspect, the invention is directed to methods for making improved vaccines. Such vaccines may be used advantageously in prophylactic and therapeutic regimens designed to elicit an immune response in a subject to whom a vaccine of the invention is administered.

### BACKGROUND OF THE INVENTION

Several publications and patent documents are referenced in this application in order to more fully describe the state of the art to which this invention pertains. The disclosure of each of these publications and documents is incorporated by reference herein.

Vaccination and immunization are terms that, in general, refer to the introduction of a non-virulent agent into a subject, which agent is capable of eliciting the subject's immune system to mount an immunological response against a pathogenic challenge. The immune system identifies invading “foreign” compositions and agents primarily by identifying proteins and other large molecules that are not normally present in the individual. The foreign protein or other large molecule may, therefore, be considered an exogenous molecule with respect to the subject. Such molecules are antigenic or immunogenic targets capable of inducing an immune response in immunocompetent subjects.

The immune system is generally viewed as having two major branches, one of which comprises humoral responses and the other comprises cellular responses. Both of these arms of the immune system are known to participate in antigen recognition and elimination. Briefly, the humoral response involves B cells which produce antibodies that are immunologically specific for a particular antigen. The cellular immune response comprises two branches: helper T (Th) cells, which elicit engagement of additional immune cells, including B cells, via direct cellular interaction and by producing cytokines that promote such engagement; and cytotoxic T lymphocytes (CTLs), which are capable of recognizing antigens and targeting a cell or particle to which the antigen is attached for destruction. CTLs are also capable of synthesizing a battery of cytokines, which are typically involved in cell mediated immune responses.

Vaccination has proven to be a successful means for conferring immune protection against many human and animal pathogens. In the search for safe and effective vaccines for immunizing individuals against infective pathogenic agents such as viruses, bacteria, and infective eukaryotic organisms, many strategies have been employed. Typically, such strategies are directed to protecting the individual from pathogenic infection and involve administering a target protein associated with the pathogen to the individual, so as to elicit an immune response to the exogenous pathogen protein. Thus, when the individual is subsequently challenged by the infective pathogen, the individual's immune system recognizes the target protein and mounts an effective defense against the pathogen. There are several vaccine strategies for presenting pathogen proteins that include presenting the protein as part of a non-infective or less infective agent or as a discreet protein composition.

The citation of references herein shall not be construed as an admission that such is prior art to the present invention.

### SUMMARY OF THE INVENTION

The present inventors have made the surprising discovery that defective interfering (DI) particles may be used to promote enhanced immune responses to a variety of pathogenic agents (e.g., viruses, bacteria, and infective eukaryotic organisms) and aberrant host cells (e.g., tumor cells) in vertebrate animals. DI particles are viral replication byproducts that comprise incomplete genomes capable of replicating only in the presence of complementary standard virus. As described herein, the presence of DI particles in combination with a sufficient amount of complementary standard virus enhances dendritic cell maturation and response to antigenic stimuli, which in turn leads to a more robust immune response.

In accordance with this discovery, the present invention is directed to methods for enhancing immune responses in vertebrate animals, whereby DI particles are utilized to promote dendritic cell maturation/activation. Such DI particles may be genetically engineered particles or isolated from viral stocks. In accordance with the present invention, DI particles and compositions thereof are used as natural, biodegradable adjuvants in a variety of vaccination protocols as described herein. Moreover, a composition of DI particles, in conjunction with a sufficient amount of complementary standard virus, may also serve as a natural, biodegradable adjuvant.

The present invention encompasses a method for stimulating an immune response in a subject, comprising administering to a subject at least one antigen, wherein the at least one antigen is administered in conjunction with a defective interfering (DI) particle-enriched viral population, and wherein the at least one antigen and the DI particle-enriched viral population are administered in an effective amount capable of inducing an antigen specific immune response in said subject. The present invention also encompasses use of the at least one antigen and a defective interfering (DI) particle-enriched viral population in the preparation of a medicament for inducing or promoting an antigen specific immune response in a subject.

The at least one antigen may be presented in the form of a live or attenuated infectious agent, such as a parasite, fungus, bacteria, or virus. Administering a live or attenuated infectious agent in conjunction with a DI particle-enriched viral population, wherein each is administered to a subject in an effective amount, confers protective immunity to the subject against the virulent infectious agent. Such administering may be performed for prophylactic or therapeutic purposes.

The at least one antigen may be a peptide, polypeptide, cell, cell extract, polysaccharide, polysaccharide conjugate, lipid, glycolipid, carbohydrate, virus, viral extract or a polypeptide encoded by nucleic acid. Such antigens may be isolated from an infectious agent, including without limitation a virus, bacterium, fungus, or parasite. An antigen of the invention may also be a tumor cell antigen. Such antigens may also be allergens such as those known to elicit exaggerated immune responses in subjects afflicted with allergies.

Administering an antigen(s) isolatable from an infectious agent in conjunction with a DI particle-enriched viral population to a subject, wherein each is administered in an effective amount, confers protective immunity to the subject against an infectious agent from which the antigen(s) is isolated. Such administering may be performed for prophylactic or therapeutic purposes.

Administering a tumor cell antigen in conjunction with a DI particle-enriched viral population to a subject, wherein each is administered in an effective amount, confers upon the subject an enhanced ability to mount an immune response to cells displaying the tumor cell antigen on their surface. With respect to a subject afflicted with a neoplastic disorder such administering enhances the ability of the subject's immune cells to recognize and destroy cells expressing the tumor cell antigen.

Administering an allergen in conjunction with a DI particle-enriched viral population to a subject, wherein each is administered in an effective amount, confers upon the subject an enhanced ability to mount an immune response against the allergen that is predominantly comprised of immunoglobulin G (IgG) antibodies immunologically specific for the allergen. With respect to a subject afflicted with allergies, such administering promotes activation of Th1 cells, rather than Th2 cells, which in turn reduces IgE-mediated release of vaso-active substances from mast cells. In that mast cell activation and subsequent release of vaso-active substances is an important component of allergic responses, a reduction in mast cell activation leads to amelioration of symptoms associated with an allergic response.

In another embodiment, the present invention is directed to a method for stimulating an enhanced immune response in a subject, the method comprising administering to a subject (a) a virus vaccine (e.g., a live attenuated virus vaccine); and (b) a plurality of DI particles complementary to the virus or a plurality of recombinant DI particles complementary to the virus, wherein the virus vaccine and the DI particles are administered in an effective amount capable of eliciting an enhanced immune response to the virus in the subject. The administration of a viral vaccine and a plurality of complementary DI particles or complementary recombinant DI particles may be performed in any order, and may be administered separately in either order or concomitantly. As indicated herein, the term complementary is used to refer to viruses and DI particles or recombinant DI particles that are compatible with respect to providing the machinery necessary for DI particle replication. The DI particles or recombinant DI particles of such complementary combinations may be either homologous or heterologous with respect to the virus. In accordance with the discovery of the present inventors, administration of a conventional virus vaccine supplemented with complementary DI particles or complementary recombinant DI particles serves to elicit an enhanced immune response which is more robust than that stimulated by administration of the virus vaccine alone.

Accordingly, the present invention encompasses use of (a) a virus vaccine (e.g., a live attenuated virus vaccine); and (b) a plurality of DI particles complementary to the virus or a plurality of recombinant DI particles complementary to the virus in the preparation of a medicament for eliciting an enhanced immune response to the virus in a subject.

Also encompassed herein is a method for making an improved vaccine comprising a virus vaccine (e.g., a live attenuated virus vaccine) and supplementing the virus vaccine with a plurality of DI particles complementary to the virus, wherein the ratio of DI particles to virus is at least 5: 1 to 10:1 (i.e., at least five to 10 DI particles per virus).

Subjects treatable using the methods of the invention comprise vertebrate animals, including humans.

In an aspect of the invention, a method for stimulating an immune response in a subject comprises administering to a subject at least one antigen, wherein the at least one antigen is encoded by a recombinant standard virus present in the DI particle-enriched viral population, and wherein the DI particle-enriched viral population is administered in an effective amount capable of inducing an antigen specific immune response in said subject. Recombinant standard viruses of the invention include, without limitation, any virus known or anticipated to make DI particles. A variety of viruses are known to produce DI particles. Exemplary such standard viruses of the invention include, without limitation, those viruses presented in Table 1. DI particle-enriched viral populations of the invention may be either homologous DI particle-enriched viral populations or heterologous DI particle-enriched viral populations.

Accordingly, the present invention includes the use of an at least one antigen encoded by a recombinant standard virus present in the DI particle-enriched viral population in the preparation of a medicament for inducing an antigen specific immune response in a subject.

In another aspect of the invention, the at least one antigen is encoded by a recombinant DI particle present in the DI particle-enriched viral population. Exemplary recombinant DI particles include those derived from a recombinant standard virus capable of making DI particles, such as, for example, those listed in Table 1. Alternatively, a recombinant DI particle encoding at least one antigen may be genetically engineered.

In yet another embodiment, the present invention is directed to a method for stimulating an immune response in a subject, comprising administering to a subject a homologous or heterologous DI particle-enriched viral population, wherein said DI particle-enriched viral population comprises a recombinant standard virus encoding at least one antigen and complementary DI particles (e.g., recombinant DI particles), and said homologous or heterologous DI particle-enriched viral population is administered in an effective amount capable of inducing an antigen specific immune response in said subject. In an aspect of this method, the recombinant standard virus encoding the at least one antigen is a recombinant standard virus capable of complementing DI particles. Exemplary standard recombinant viruses of the invention include recombinant paramyxovirus and recombinant orthomyxovirus. In a particular aspect, the at least one antigen is a peptide or a polypeptide. Such antigens may be isolatable from an infectious agent, including without limitation a virus, bacterium, fungus, or parasite. An antigen of the invention may also be a tumor cell antigen or an allergen.

The present invention further encompasses the use of a homologous or heterologous DI particle-enriched viral population, wherein said DI particle-enriched viral population comprises a recombinant standard virus encoding at least one antigen and complementary DI particles (e.g., recombinant DI particles), in the preparation of a medicament for inducing an antigen specific immune response in a subject.

Administering a heterologous DI particle-enriched viral population, which comprises a recombinant standard virus encoding at least one antigen of an infectious agent and recombinant DI particles, in an effective amount to a subject confers protective immunity to the subject against the infectious agent from which the antigen(s) is isolatable. Such administering may be performed for prophylactic or therapeutic purposes. Subjects treatable using this method of the invention comprise non-human animals, including vertebrate animals, and humans.

Administering a heterologous DI particle-enriched viral population which comprises a recombinant standard virus encoding a tumor cell antigen and recombinant DI particles in an effective amount to a subject confers upon the subject an enhanced ability to mount an immune response to cells displaying the tumor cell antigen on their surface. With respect to a subject afflicted with a neoplastic disorder, such administering enhances the ability of the subject's immune cells to recognize and destroy cells expressing the tumor cell antigen.

The invention also includes a method for stimulating an immune response in a subject, comprising administering to a subject a packaging defective recombinant virus derived from a virus capable of generating DI particles, wherein said packaging defective recombinant virus comprises a complementary antigenomic promoter and a nucleic acid sequence encoding at least one antigen, and said packaging defective recombinant virus is administered in an amount capable of inducing an antigen specific immune response in said subject.

For in vivo use, the packaging defective recombinant virus is delivered via a virus-like particle (VLP) by expressing a construct encoding the packaging defective recombinant virus in a packaging cell line. Such a cell line may express Sendai virus envelope proteins, for example, or envelope proteins from other viruses suitable for encapsulating the packaging defective recombinant virus. A cell line expressing Sendai HN, F and M envelope proteins, for example, may be used to encapsulate a defective recombinant virus within a viral envelope that will serve as a vehicle for delivery to the particular cell or cell type. Alternatively, a packaging cell that expresses envelope proteins of another suitable virus (e.g., VSV or NDV) may be used. Packaging cell lines may also express a targeting molecule designed to deliver the defective recombinant virus with greater specificity to a particular cell or cell type. Such targeting molecules are incorporated into the envelope of VLPs generated in these packaging cell lines. A protein such as an antibody or portion thereof (e.g., an Fv-like domain) may, for example, be incorporated into the viral envelope to target the defective recombinant virus more effectively.

Also encompassed is the use of a packaging defective recombinant virus derived from a virus capable of generating DI particles, wherein said packaging defective recombinant virus comprises a complementary antigenomic promoter and a nucleic acid sequence encoding at least one antigen, in the preparation of a medicament for inducing an antigen specific immune response in a subject.

In a particular aspect, the invention is directed to a method for stimulating an immune response in a subject comprising administering to a subject a packaging defective recombinant paramyxovirus, wherein the packaging defective recombinant paramyxovirus comprises a complementary antigenomic promoter and a nucleic acid sequence encoding at least one antigen, and said packaging defective recombinant paramyxovirus is administered in an amount capable of inducing an antigen specific immune response in the subject. In a particular aspect, the packaging defective recombinant paramyxovirus is a packaging defective recombinant Sendai virus. In another aspect of the method, a packaging defective recombinant orthomyxovirus is used, wherein the packaging defective recombinant orthomyxovirus comprises a complementary antigenomic promoter and a nucleic acid sequence encoding at least one antigen, and the packaging defective recombinant orthomyxovirus is administered in an amount capable of inducing an antigen specific immune response in said subject. This approach may also be applied to any of the other paramyxoviruses upon development of a recombinant system, examples of which include New Castle Disease virus and Respiratory Syncytial Virus.

Also presented is a method for activating a dendritic cell, comprising contacting a dendritic cell with at least one antigen in conjunction with a DI particle-enriched viral population, in an amount effective to activate the dendritic cell, wherein the method is performed ex vivo. The method may further comprise administering the ex vivo activated dendritic cell to a subject as described herein below. Use of an at least one antigen in conjunction with a DI particle-enriched viral population in the preparation of a medicament for activating a dendritic cell is also envisioned.

In one aspect, the invention encompasses a method comprising isolating DCs from a subject and treating these DCs ex vivo with a DI particle-enriched viral population (e.g., a DI particle-enriched Sendai virus population) in combination with an antigen of interest and re-introducing these activated, mature DCs back into the subject. Ex vivo DCs may be obtained directly from blood or other tissue and purified using standard techniques or grown from blood monocytes or bone marrow in the presence of appropriate growth factors. Once the cells are isolated they can be infected with the DI particle-enriched viral population. Recombinant DI particle-enriched viral populations that are engineered to express the antigen of interest may also be used in this aspect of the invention. Re-introduction of such matured DCs confers upon the subject an enhanced ability to mount an immune response against the antigen of interest. Antigens of utility for such purposes are described in detail herein and include, without limitation, antigens isolatable from an infectious agent, tumor cell antigens, and allergens.

In another embodiment, a method comprising isolating DCs from a subject and treating these DCs ex vivo with a plurality of recombinant packaging defective DI particles (e.g., a plurality of recombinant packaging defective Sendai virus DI particles) in combination with an antigen of interest and re-introducing these activated, mature DCs back into the subject is described. Such a recombinant defective DI particle may alternatively be engineered to express the antigen of interest. Re-introduction of such matured DCs confers upon the subject an enhanced ability to mount an immune response against the antigen of interest. Antigens of utility for such purposes are described in detail herein and include, without limitation, antigens isolatable from an infectious agent, tumor cell antigens, and allergens. This aspect of the invention may be used to particular advantage with immunocompromised patients because the defective DI particles are not capable of producing any infectious viral particles.

In another embodiment, a method comprising isolating DCs from a subject and transfecting these DCs (ex vivo or in vivo) with a plurality of recombinant packaging defective DI constructs (e.g., a plurality of recombinant packaging defective Sendai virus DI constructs), wherein the recombinant packaging defective DI constructs comprise a nucleic acid sequence encoding an antigen of interest, and re-introducing these activated, mature DCs back into the subject is described. Re-introduction of such matured DCs confers upon the subject an enhanced ability to mount an immune response against the antigen of interest. Antigens of utility for such purposes are described in detail herein and include, without limitation, antigens isolatable from an infectious agent, tumor cell antigens, and allergens. This aspect of the invention may be used to particular advantage with immunocomprorised patients because the defective DI constructs are not capable of producing any infectious viral particles and the presence of packaging proteins (e.g., capsid proteins) is eliminated. Accordingly, administration of such activated DCs can be performed multiple times without encountering complications associated with an immune response generated against viral packaging proteins. Use of such activated DCs in the preparation of a medicament for enhancing an ability to mount an immune response against a particular antigen of interest in a subject is also included.

The invention also encompasses a method for maturing DCs ex vivo, wherein DCs and cancer cells (e.g., tumor cells) are isolated from a patient suffering from a cancer and contacting the DCs ex vivo with isolated cancer cells infected with a DI particle-rich virus population (e.g., a DI particle-rich Sendai virus population) or transfected with a recombinant defective construct as described herein. Mature ex vivo treated DCs are then re-introduced into the patient wherein they promote a robust immune response directed against the cancer cells.

The invention also encompasses a mixture of a DI particle-enriched viral population and a conventional vaccine. Such a conventional vaccine may be a subunit vaccine, a recombinant live viral-delivery vector, a recombinant live bacterial vaccine-delivery vector, a nucleic acid vaccine, virus-like particles (VLPs), a modified virus vaccine, an inactivated virus vaccine, or a live attenuated virus vaccine. Compositions comprising such a mixture and a pharmaceutically acceptable carrier are also envisioned. Such mixtures and compositions thereof may be used in the preparation of a medicament for promoting augmented response to a conventional vaccine.

The invention is also directed to heterologous DI particle-enriched viral populations. In a broad aspect of the invention, a heterologous DI particle-enriched viral population comprises standard virus of a first virus strain and DI particles of a second virus strain, wherein the DI particles are complemented by the first virus strain. In a particular embodiment, a heterologous DI particle-enriched viral population comprises standard virus of a first paramyxovirus strain and DI particles of a second paramyxovirus strain. In a different embodiment, a heterologous DI particle-enriched viral population comprises standard virus of a first orthomyxovirus strain and DI particles of a second orthomyxovirus strain. Also envisioned are compositions comprising heterologous DI particle-rich viral populations of the invention and pharmaceutically acceptable carriers. Methods of using heterologous DI particle-rich viral populations of the invention and compositions thereof are also described herein. Use of such heterologous DI particle-rich viral populations and compositions thereof in the preparation of a medicament for inducing an immune response in a subject are also envisioned.

Recombinant standard viruses and recombinant DI particles derived therefrom and compositions thereof are also encompassed by the present invention, as are methods of using same. Moreover, DI particle-rich viral populations derived from recombinant standard viruses of the invention and compositions thereof are also included in the present invention, as well as methods of using such DI particle-rich viral populations. Use of recombinant standard viruses and recombinant DI particles derived therefrom and DI particle-rich viral populations derived from recombinant standard viruses and compositions of any of the aforementioned in the preparation of a medicament for promoting an immune response in a subject are also foreseen.

In an aspect of the invention, recombinant standard viruses that comprise a nucleic acid sequence encoding a polypeptide of interest (e.g., an exogenous antigenic polypeptide) and genetically engineered DI particles derived from such standard viruses are envisioned. In a particular embodiment, the invention encompasses recombinant Sendai virus that is engineered to comprise a nucleic acid sequence encoding an antigenic polypeptide of interest. DI particle-rich virus populations of such recombinant Sendai viruses can be generated as described herein and administered to a subject to induce a potent immune response against the encoded polypeptide.

The present invention also presents recombinant packaging defective viruses derived from a virus capable of making DI particles (such as those listed in Table 1), wherein the recombinant packaging defective virus comprises a complementary antigenomic promoter. Exemplary recombinant packaging defective viruses of the invention include paramyxoviruses or orthomyxoviruses comprising a complementary antigenomic promoter. Such recombinant packaging defective viruses may further comprise a nucleic acid sequence encoding an antigen. Also encompassed are recombinant packaging defective paramyxovirus or orthomyxovirus constructs comprising a complementary antigenomic promoter. Such recombinant packaging defective viral constructs may further comprise a nucleic acid sequence encoding an antigen. Compositions comprising recombinant packaging defective paramyxovirus or orthomyxovirus comprising a complementary antigenomic promoter or recombinant DI particles derived therefrom and a pharmaceutically acceptable carrier are also included. Compositions comprising recombinant packaging defective paramyxovirus or orthomyxovirus constructs comprising a complementary antigenomic promoter and a pharmaceutically acceptable carrier are also envisioned.

In an aspect of the present invention, a method is presented for making improved vaccine compositions, administration of which confers protection against infective pathogenic agents (such as, e.g., viruses, bacteria, and infective eukaryotic organisms) to susceptible vertebrates. Such improved vaccine compositions may be made by combining, for example, at least one antigen with a DI particle-rich viral population. The at least one antigen may be provided as a polypeptide, for example, or as a nucleic acid sequence encoding the polypeptide. Complex antigenic compositions comprising, for example, a live attenuated virus, killed virus, or whole cell (e.g., an irradiated tumor cell) are also envisioned for administration in conjunction with a DI particle-rich viral population. Such improved vaccine compositions may also comprise, for example, recombinant viruses capable of making DI particles that have been engineered to express at least one antigen of interest and DI particles derived therefrom and genetically engineered DI constructs. As detailed herein, paramyxoviruses and orthomyxoviruses capable of generating DI particles are exemplary viruses of the invention.

Methods described herein and improved vaccine compositions of the present invention may be used to modulate a subject's immune system by stimulating a humoral immune response, a cellular immune response, or a combination thereof. As used herein, a subject may refer without limitation to: humans, primates, horses, cows, sheep, pigs, goats, dogs, cats, avian species, fish, and rodents.

The present invention is also directed to a method for screening to identify an agent capable of modulating retinoic acid inducible gene I (RIG-I) activity, wherein the method comprises contacting RIG-I molecules with a plurality of agents, wherein each of the agents is contacted individually with RIG-I, and wherein a change in RIG-I activity in the presence of an agent identifies the agent as a RIG-I modulator. In accordance with the invention, the contacting may occur in a test tube, a cell, or in an animal. RIG-I modulators so identified may increase or decrease RIG-I activity.

The invention also encompasses agents identified using these methods and compositions thereof, as well as methods of use for same.

Other objects and advantages will become apparent to those skilled in the art from a review of the following description which proceeds with reference to the following illustrative drawings.

### BRIEF DESCRIPTION OF THE DRAWINGS

FIGS. 1A-F show histogram bar graphs (A, B, D, E, F) and an immunoblot (C) revealing that enhanced type I IFN induction ability of SeV-C is not due to the absence of a functional type I IFN antagonist. (A) mRNA was extracted from SeV-infected DC2.4 cells at 1, 3, 6, 24, or 48 h post infection. Reverse transcription and quantitative PCR (qPCR) using primers for IFN α and β were performed. (B) Cytokines were detected by ELISA in the supernatants of DC2.4 cells infected with SeVs for 24 h. (C) NIH-3T3 cells were infected with SeV-52 or SeV-C for 6 h or mock infected (Ni). Cells were subsequently treated with IFNβ or mock treated and whole cell extracts were immunoblotted for STAT1 and phosphorylated STAT1. (D-F) qRT-PCR analysis was performed for IFNβ on mRNA extracted from DC2.4. Error bars represent standard deviation of triplicate measurements. (D) Cells were infected with indicated MOIs of SeV-52 and/or SeV-C. (E) Cells were pre-infected with SeV-52 for 18 h and subsequently infected with indicated MOIs of SeV-C. (F) Cells were infected with influenza PR8 and/or ΔNS1 at the indicated MOIs for 24 h. Results are representative of three or more experiments.

FIGS. 2A-B show histogram bar graphs depicting that enhanced type I IFN induction ability of SeV-C correlates with higher dsRNA compared to SeV-52. (A) 293T cells were cotransfected with the influenza NS1 protein dsRNA binding domain (amino acids 1-73) or empty vector and a luciferase reporter construct driven by the IFNβ promoter. Cells were infected with SeVs for 24 h and whole cell lysates were tested for luciferase activity. (B) DC2.4 cells were infected with SeV-52 or SeV-C MOI 5 for 3, 6, or 24 h. Reverse transcription of extracted mRNA and qPCR using primers for a region of the NP gene that is identical in both SeVs were performed. Error bars represent standard deviations from triplicate wells in each experiment. Results are representative of three or more experiments.

FIGS. 3A-C show a flow cytometry histogram (A) and histogram bar graphs (B,C) showing that DI particles contribute to the enhanced induction of DC maturation and type I IFN by SeV-C. BM-DCs were infected with various SeV preparations. (A) Cells infected for 24 h were labeled with FITC-conjugated antibodies for CD80 or CD86 and analyzed by flow cytometry. The histograms show an overlay of the isotype control (filled), the mock infected cells (dotted line), and the cells infected with different viruses (black line). The numbers indicate the percentage of cells in the infected cultures showing high surface expression of the marker. (B) Cytokine ELISA was performed on cellular supernatants 24 hours post infection (hpi) for IL-6, TNFα, IL-12p40 and IFNα. Error bars represent standard deviations of triplicate measurements. (C) RNA was extracted 6 h after infection for qRT-PCR analysis of NP gene expression and 24 h after infection for IL-12p35 analysis. Results are representative of more than five experiments.

FIGS. 4A-B are histogram bar graphs showing that DI particles utilize RIG-I in enhancing cellular stimulation by virus infection. (A, B) 293T cells were co-transfected with empty vector, or vector expressing RIG-I or RIG-IC along with a luciferase construct driven by the IFNβ promoter. Cells were infected with standard SeV-C (A, B) or SeV-C low DI (B) for 24 h and whole cell lysates were tested for luciferase activity. Error bars depict standard deviation of triplicate wells in each experiment and results are representative of three experiments.

FIGS. 5A-C show a photograph of an ethidium bromide stained agarose gel (A), a histogram bar graph (B), and a flow cytometry histogram (C) revealing that DI particles enhance the ability of SeV-52 to induce DC maturation. (A) RNA was extracted from SeV-52 or SeV-52 hi DI preparations and reverse transcribed using a primer specific for the SeV antigenomic promoter. Lanes 1 and 3 show PCR products obtained using one primer against the SeV antigenomic promoter (1 primer) indicative of DI particles. Lanes 2 and 4 show amplification of a 3400 bp fragment of the L gene as a positive control for the presence of standard virus genome (2 primers). Arrows indicate amplified copy-back DI particles. (B) BM-DCs were infected with SeV-52 or SeV-52 hi DI preparations at a MOI of 0.5 for 24 h. Supernatants from infected cells were analyzed by BLISA for IL-6 and IL-12. Error bars represent standard deviation of triplicate measurements in each ELISA. (C) The infected cells were also analyzed for surface expression of CD80 and MHC II by flow cytometry. The histograms show an overlay of the isotype control (filled), the mock infected cells (dotted line), and the cells infected with different viruses (black line). The indicated numbers are the percentage of cells showing high surface expression of the marker. Results are representative of three experiments.

FIGS. 6A-E show an electron micrograph (A), histogram bar graphs (B-D), and a flow cytometry histogram (E) demonstrating that pDI particles enhance DC maturation in a replication dependent manner. (A) SeV-C and pDI particles were visualized by transmission electron microscopy. (B) BM-DCs were infected with SeV-C low DI, SeV-C low DI plus increasing doses of pDI particles (5, 10, 50, 100, 200, or 500 HA units), or standard SeV-C (5, 10, 50, 100, or 200 HA units). (B) Cellular RNA was extracted 6 hpi and analyzed by qRT-PCR for production of viral NP mRNA. Error bars represent standard deviation of duplicate wells. Cytokine ELISAs for IFNα (C), IL-12p40, TNFα, and IL-6 (D) were performed on cellular supernatants. Error bars represent standard deviation of duplicate (C) or triplicate (D) measurements in each ELISA. (E) Flow cytometry for MHC II or CD80 was performed on cells infected with an MOI of 1.5 with the indicated virus and/or treated with 200HA units of pDI particles. The histograms show an overlay of the isotype control (dotted line), the mock infected cells (grey filled), and the cells infected with different viruses (black filled). The indicated numbers are the percentage of infected cells showing high surface expression of the marker. Results are representative of more than 5 experiments.

FIGS. 7A-C show flow cytometry histograms (A, B) and histogram bar graphs (C) revealing that pDI particles enhance the ability of DCs to prime naive CD8⁺ T cells. BM-DCs incubated with OVA SIINFEKL peptide (SEQ ID NO: 18) and infected with SeV-C low DI alone or in the presence of 100 HA units of pDI particles were cultured at a 1:10 or 1:20 ratio with CFSE-labeled TCR transgenic OT-I cells for 3 days. (A) Proliferation of OT-I cells after 3 days of culture with BM-DCs treated as indicated. (B) Proliferation and expression of CD25 and CD69 by OT-I cells incubated with DCs treated as indicated in the presence of SIINFKL peptide (SEQ ID NO: 18). Cells are gated on CD8⁺ T cells (90% of the culture). The numbers correspond to the percentage of CD8⁺ T cells expressing the marker. (C) Cytokine secretion measured by multiplex analysis. Error bars represent standard deviation and the asterisk indicate a p<0.05. Results are representative of three experiments.

FIG. 8 shows a schematic diagram of a packaging defective recombinant Sendai virus.

FIG. 9 shows a schematic diagram of a reverse genetic system for the generation of DI particles.

FIG. 10 shows a cartoon of downstream signaling pathways involved in cDC maturation upon virus infection. Virus infection triggers induction of both type I IFN and DC maturation through the activation of transcription factors such as IRF3 and NF-κB. DC maturation is characterized by the up-regulation of MHC and costimulatory molecules as well as by the secretion of pro-inflammatory cytokines such as IL-12. Complete maturation of cDCs in response to virus does not rely on secreted type I IFN or TLR signaling. Concomitant activation of the type I IFN induction pathway and DC maturation pathway does, however, suggest that dsRNA-binding molecules known to be involved in type I IFN induction, such as RIG-I or mda-5, may also lead to DC maturation, while the dsRNA-binding protein PKR has been shown to be unnecessary for complete DC maturation in response to live viruses (gray signaling pathways are dispensable for complete DC maturation).

FIGS. 11A-E show histograms depicting results generated following flow cytometry.

FIGS. 12A-G presents nucleic and amino acid sequences of human and mouse RIG-I.

FIGS. 13A-AM presents amino acid sequences corresponding to a variety of tumor cell antigens and allergens.

FIGS. 14A-C show histogram bar graphs (A) and flow cytometry histograms (B, C) demonstrating that human monocyte-derived DCs mature more efficiently upon infection by a SeV having a high content of DI particles.

FIGS. 15A-B show histogram bar graphs revealing that DCs infected with SeV in the presence of DI particles induce more efficient secretion of IFN gamma from T cells than DCs infected in the absence of DI particles.

FIGS. 16A-B show histogram bar graphs signifying that DI particles purified from SeV-C enhance DC maturation ability of complementary viral strains in a Type I IFN independent manner.

FIGS. 17A-D show histogram bar graphs demonstrating that enhancement of DC maturation provided by DI particles requires virus and DI particle replication.

FIGS. 18A-N show flow cytometry histograms depicting the effects of ex vivo activated DCs.

### DETAILED DESCRIPTION OF THE INVENTION

The present invention is based on the surprising discovery that defective interfering (DI) particles may be used to enhance or augment immune responses in vertebrate animals. As described herein, the presence of DI particles enhances dendritic cell maturation and response to antigenic stimuli, which in turn leads to a more robust immune response. These findings stand in marked contrast to prevailing thought in the field of vaccine development, for example, which views DI particles as undesirable components of vaccine compositions, the presence of which should be reduced or ideally eliminated.

A variety of viruses are known to produce DI particles. As described herein and known in the art, DI particles comprise incomplete viral genomes that replicate only in the presence of standard virus. The present invention encompasses the use of any virus capable of generating DI particles. Exemplary such viruses of the invention include, without limitation, paramyxoviruses and orthomyxoviruses. A partial list of viruses capable of producing DI particles is presented in Table 1.

Table 1 presents a non-limiting list of viruses known to make DI particles.

| Virus Family | Virus | Host |
|---|---|---|
| Paramyxoviridea: | Sendai virus | mouse |
| | Human Parainfluenza virus | human |
| | Measles virus | human |
| | Respiratory syncytial virus | human |
| | Simian virus 5 | monkey |
| | Newcastle disease virus | birds |
| | Mumps | human |
| Togaviridea | Sindbis virus | |
| | Semliki Forest virus | |
| Orthomyxoviridea: | Influenza | vertebrates |
| Picornaviridea: | Foot and Mouth disease virus | human |
| | Poliovirus | human |
| | Hepatitis A | human |
| Rhabdoviridae: | Vesicular Stomatitis virus | mammals, insects |
| | Rabies | dogs, cats, |
| | | bats, human, etc. |
| Flaviviridae: | Hepatitis C | human |
| | Classical Swine fever virus | pigs |
| | West Nile virus | human, birds, |
| | | other animals |
| | Dengue virus | human |
| | Yellow Fever Virus | human |
| Coronaviridae: | Severe acute respiratory | |
| | syndrome (SARS) | |
| Reoviridae: | | mammals, plants |
| Retroviridae: | HIV | human |
| Hepadnaviridae | Hepatitis B | human, chimps |
| Bunyaviridae: | Rift valley fever virus | human, animals |
| Unclassified: | Hepatitis delta | |

Viruses capable of generating DI particles can, moreover, be propagated so as to increase the number of DI particles in a viral population produced. Multiple undiluted passages of Sendai virus (SeV) strains, for example, result in enhanced DI particle production. In general, DI particles enrich their proportion in co-infected cells by replicating faster than standard virus genomes due to their smaller length and differential promoter efficiencies [Calain & Roux. Virology 212, 163-73 (1995); Tapparel & Roux. Virology 225, 163-71 (1996)]. DI particles may be purified by glycerol centrifugation or on sucrose gradients using standard protocols known in the art.

As described herein, the present inventors have made the surprising discovery that a viral population comprising both standard viral particles and DI particles, wherein the ratio of DI particles is enriched relative to that of the standard viral particles, can elicit a more robust immune response than a similarly composed viral population, wherein the ratio of DI particles is not enriched relative to that of the standard viral particles. In view of the above, the present inventors are the first to appreciate that enrichment of DI particles relative to standard virus in a viral vaccine, for example, produces an improved vaccine having superior properties with which to promote enhanced immune responses. This is a particularly surprising finding when considered in light of the prevailing opinion in the field that the presence of DI particles in a viral vaccine reduces vaccine efficacy via DI particle mediated competition with, for example, a live, attenuated virus in such a vaccine. Along these lines, several U.S. Pat. No. (e.g., 5,980,901; 5,847,096; and 5,716,821) propose the use of DI particles as anti-viral agents that act by successfully competing with various pathogenic viruses (Hepatitis B virus, Human Immunodeficiency Virus, and Respiratory Syncytial virus, respectively), thereby reducing pathogenic viral load.

### DEFINITIONS

The following terms are defined as used herein. Terms not defined should be interpreted as is usual and customary in the fields of molecular biology and virology.

As used herein, the term “subject” is used to refer to any member of the subphylum Vertebrata. The term “vertebrate” is intended to encompass its normal meaning and thereby refers to any animal having an internal skeleton made of bone, including a backbone. Vertebrate animals include: amphibians, birds, fish, reptiles, and mammals.

The terms “mammal” and “mammalian” are intended to encompass their normal meaning. While the invention is most desirably intended for efficacy in humans and other primates, it is also applicable to domestic mammalian species, including without limitation, dogs, cats, cows, horses, pigs, sheep, goats, mice, rabbits, and rats, and fish, etc. and birds, for example, chickens, turkeys, and domestic waterfowl.

As used herein, the term “antigen” is used to refer to any substance that causes a subject's immune system to generate an immune response (e.g., to produce antibodies) against the substance. The antigen may be a foreign substance from the environment (such as, e.g., chemical, bacteria, virus, or pollen) or formed within the body (such as, e.g., a bacterial toxin or a substance produced by a cell and/or tissue).

As used herein, the term “allergen” is a substance that can elicit an allergic reaction. Allergens are substances that can cause mild to life-threatening allergic responses in individuals with allergies. Common allergens include: plants; drugs (e.g., antibiotics and serums); foods (e.g., milk, chocolate, strawberries, wheat, and nuts); infectious bacteria; viruses; animal parasites; and inhalants (e.g., dust, pollen, perfumes, and smoke).

By “immune response” or “immunity” as the terms are interchangeably used herein, is meant the induction of a humoral (i.e., B cell) and/or cellular (i.e., T cell) response. Suitably, a humoral immune response may be assessed by measuring the antigen-specific antibodies present in serum of immunized animals in response to introduction of a vaccine of the invention into the host. In an exemplary embodiment, the immune response is assessed by the enzyme linked immunosorbant assay of sera of immunized mammals, or by microneutralization assay of immunized animal sera. A CIL assay can be employed to measure the T cell response from lymphocytes isolated from the spleen of immunized animals.

The terms “enhanced immune response” or “enhanced immunity” are used herein to refer to an immune response or immunity characterized by an increase in immune cell activation in response to an antigen and/or pathogen, the rapidity with which the immune system recognizes and mounts a detectable response to an antigen and/or pathogen, and/or an increase in the duration of the response of the immune system to an antigen and/or pathogen. The duration of an immune response may refer to both short term immunity (e.g., duration of the instant response to an antigenic stimulus) and long term immunity (the ability to mount an effective immune response following a subsequent encounter with the antigenic stimulus).

With respect to antibodies, the term “immunologically specific” refers to antibodies that bind to one or more epitopes of a protein of interest, but which do not substantially recognize and bind other molecules in a sample containing a mixed population of antigenic biological molecules.

The term “vaccine” or “vaccine composition” refers to a composition comprising at least one antigen which, upon inoculation into a subject, induces an immune response specific for that antigen or a cell or organism expressing the antigen and thereby confers protective immunity to the vaccinated subject against the antigen or a cell or organism expressing the antigen. With respect to a “vaccine” or “vaccine composition” comprising, e.g., a live, attenuated virus, inoculation into a subject induces a complete or partial immunity to the pathogenic version of the virus, and/or alleviates the symptoms of disease caused by pathogenic versions of the virus. The protective effects of a vaccine composition against a virus are normally achieved by inducing in the subject an immune response, either a cell-mediated or a humoral immune response, or a combination of both. Generally speaking, abolished or reduced incidence of viral infection, amelioration of symptoms, or accelerated elimination of the viruses from infected subjects are indicative of the protective effects of the vaccine composition.

A therapeutically effective amount or dose refers to that amount of a compound sufficient to result in a healthful benefit in the treated subject. Amounts effective for this use will depend on the purpose of the intervention (e.g., therapeutic or prophylactic), the severity of disease if the subject is afflicted by a disease and the weight and general state of the patient.

An “effective amount” of live-virus or subunit vaccine, for example, can be administered to a subject (human or animal) alone or in conjunction with an adjuvant (e.g. as described in U.S. Pat. No. 5,223,254 or Stott et al., (1984) J. Hyg. Camb. 251-261) to induce an active immunization against a pathogenic infection. An effective amount is an amount sufficient to confer immunity against the pathogen and can be determined by a skilled practitioner using routine experimentation. In the case of influenza vaccine, for example, 15 μg of antigen has been determined to be an effective amount.

Determination of a therapeutically effective amount may take into account such factors as the weight and/or age of the subject and the selected route for administration. Vaccines can be administered by a variety of methods known in the art. Exemplary modes include oral (e.g., via aerosol), intradermal, intramuscular, intraperitoneal, intravenous, subcutaneous, parental, transdermal and intranasal routes. If necessitated by a particular mode, the vaccine may be encapsulated.

The term “viruses”, “viral isolates” or “viral strains” as used herein refers to viral particles or virions that contain viral genomic DNA or RNA, associated proteins, and other chemical constituents (such as lipids). By “nucleic acid molecule encoding a virus” or “nucleic acid molecule of a virus” is meant the genomic nucleic acid sequence of the virus, either in the form of RNA or DNA.

The term “attenuation” is used to refer to a virus that has lost some or all of its ability to proliferate and/or cause disease in a subject infected with the virus. An attenuated virus can, for example, be a virus that is unable to replicate or is limited to one or a few rounds of replication, or restricted in cell or tissue tropism, when present in a subject in which a wild type pathogenic version of the attenuated virus is competent to replicate.

An attenuated virus may have one or more mutations in a gene or genes that are involved in pathogenicity of the virus. Such mutations are also referred to herein as “attenuating mutation(s)”. An attenuated virus can be produced from the wild type, pathogenic virus by UV irradiation, chemical treatment, or high serial passage of the wild type, pathogenic virus in vitro. Alternatively, an attenuated virus can be produced from wild type, pathogenic virus by deleting viral sequences known to confer virulence, insertion of sequences into the viral genome, or making one or more point mutations in the viral genome. An attenuated virus can be a viral isolate obtained from an animal, which isolate is derived from the wild type, pathogenic version of the virus through events other than artificial means, e.g., events that have occurred in a host animal such as recombination.

By “mutation” is meant to include deletion, insertion or substitution of one or more nucleotides, or a combination thereof. In accordance with the present invention, the mutation preferably confers a desirable phenotype, for example attenuation of virulence, alteration of cellular tropism or biotype, alteration of species tropism, or expression of a foreign gene cassette.

The terms “prime” and “boost” are intended to have their ordinary meanings in the art. “Priming” refers to immunizing with a first composition, which induces a higher level of immune response to the antigen upon subsequent immunization or “boosting” with the same or another composition, than the immune response obtained by immunization with a single vaccine composition, e.g., the priming composition alone or the boosting composition alone.

A vaccine may comprise a live attenuated virus in a suitable pharmaceutically-, or physiologically acceptable carrier, such as isotonic saline or isotonic salts solution. The appropriate carrier will be evident to those skilled in the art and will depend in large part upon the route of administration. Alternatively, vaccines composed of polynucleotide molecules desirably contain optional polynucleotide facilitating agents or “co-agents”, such as a local anaesthetic, a peptide, a lipid including cationic lipids, a liposome or lipidic particle, a polycation such as polylysine, a branched, three-dimensional polycation such as a dendrimer, a carbohydrate, a cationic amphiphile, a detergent, a benzylammonium surfactant, or another compound that facilitates polynucleotide transfer to cells. Non-exclusive examples of such facilitating agents or co-agents useful in this invention are described in U.S. Pat. Nos. 5,593,972; 5,703,055; 5,739,118; 5,837,533; International Patent Application No. WO96/10038, published Apr. 4, 1996; and International Patent Application No WO94/16737, published Aug. 8, 1994, which are each incorporated herein by reference.

As used herein, the term “a fragment” of a nucleic or amino acid sequence refers to a portion or partial domain of an indicated nucleic or amino acid sequence. When used in the context of a fragment of a larger amino acid sequence, such a fragment may comprise, for example, an antigenic epitope.

As used herein, the term “incomplete particle” shall refer to virus variants that are missing a portion of a viral genome and are replication-defective. They can be rescued by and interfere with the replication of complementary helper viruses.

As used herein, the term “defective interfering” shall refer to viral particles that are replication defective, rescuable by helper viruses, have an interference effect on wild type virus, and enrich replication defective virus. Defective interfering particles are wide-spread in many DNA and RNA viruses in bacteria, plants and animals.

As used herein, the term “replicative defective” is used to refer to a genetic element or virus that is unable to duplicate or multiply by itself. As used herein, the term “packaging defective” is used to refer to a genetic element or virus that is unable to package itself into a viral particle.

As used herein, the term “standard virus” refers to replication competent virions capable of complementing DI particles.

As used herein, the terms “complementary” or “trans complementary” are used to refer to different genetic entities, e.g., two different viruses (or strains of the same virus) or plasmids that are capable of genetically complementing or rescuing each other.

As used herein, the term “rescuability” shall mean the ability to survive when supplied with the normal functional core proteins.

The term “enrichment” is used to refer to an increase in proportion in a mixture of different viruses.

The term “DI particle-rich viral population” or “DI particle-rich viral preparation” is used herein to refer to a viral population wherein the ratio of DI particles is enriched relative to that of the standard virus present in the population. In general, and particularly with respect to in vitro applications, a DI particle-rich viral population refers to a viral population wherein the ratio of DI particles to standard virus is at least 2:1 (i.e., at least two DI particles per standard virus). For in vivo applications, wherein the presence of DI particles essentially serves the role of an adjuvant that enhances dendritic cell maturation, a DI particle-rich viral population refers to a viral population wherein the ratio of DI particles to standard virus is at least 5:1 to 10:1 (i.e., at least five to 10 DI particles per standard virus).

As used herein, the term “contacting” broadly refers to the act of bringing two or more agents or components into physical association or contact. With respect to populations of potentially infectious agents (e.g., DI particle enriched viral populations), the term contacting may be used to refer to cells brought into contact with infectious particles under conditions wherein the infectious particles can infect the cells. With respect to genetic constructs, for example, the term contacting may be used to refer to cells brought into contact with a genetic construct under conditions wherein the cells are transfected with the genetic construct or with cell homogenates (e.g., tumors) to “feed” the cells with the antigens.

As used herein, the term “homologous DI particles” or “endogenous DI particles” refers to DI particles that are derived from a first type of standard virus and are, therefore, homologous with respect to the first type (the same type) of standard virus. Sendai virus DI particles, for example, are homologous with respect to Sendai standard virus. The terms may also be used in the context of DI particles that are homologous to a first type of standard virus, but have been genetically engineered or isolated and then added to a population of a first type of standard virus. The term “homologous DI particle-rich viral population”, therefore, refers to a homologous mixture of DI particles and like standard virus.

As used herein, the term “heterologous DI particles” or “exogenous DI particles” refers to DI particles that are derived from a first type of standard virus and are, therefore, heterologous with respect to a second type (a different type) of standard virus. Sendai virus DI particles are, for example, heterologous with respect to vesicular stomatitis virus standard virus. Heterologous may also refer to different strains of related viruses, for example, Sendai Cantell and Sendai 52, or H3 influenza with an H2 influenza or influenza B. The term “heterologous DI particle-rich viral population”, therefore, refers to a heterologous mixture of DI particles and standard virus that is different from that of the DI particles.

A list of paramyxoviruses and their hosts includes, but is not limited to: Parainfluenzavirus 14 (Hurna); Sendai virus (Mouse); Bovine Parainfluenzavirus type 3 (Cows); Simian virus 5 (Monkeys); Mumps virus (Human); Newcastle disease virus (Birds); Measles virus (Human); Canine Distemper Virus (Dogs); Hendra virus (Human); Nipha virus (Human); Equine morbillivirus (Horse); Respiratory Syncytial Virus (Human); Bovine Respiratory Syncytial Virus (Cows); Avian pneumovius (Birds); Atlantic salmon paramyxovirus (ASPV; Fish; see, e.g., Kvellestad et al. J Gen Virol., 2003, 84, 2179-2189).

A non-limiting list of viruses known to be capable of making DI particles is presented in Table 1.

### Methods and Compositions for Enhancing Immune Responses

The present invention embraces a method for enhancing an immune response to an antigen, wherein the antigen is administered to a subject along with a mixture of DI particles and a small amount of replicating virus capable of supplementing essential functions missing from the DI particles (DI particle-rich viral population), such as, for example, replication machinery. Such DI particle-rich viral populations may comprise homologous and/or heterologous mixtures of DI particles and complementary standard virus. The presence of the DI particles enhances the immune response to the antigen by inducing DC maturation which in turn promotes antigen specific immunologic reactivity. Such antigens include, but are not limited to: antigenic motifs, polypeptides, and epitopes of infectious agents such as viruses, bacteria, and eukaryotic infective organisms, as well as allergens and antigens characteristic of infected or transformed host cells. These antigens may be administered as, for example, polypeptides or polypeptide complexes. Nucleic acid sequences encoding antigenic polypeptides are also envisioned as a means for expression and delivery of antigenic polypeptides. More complex antigens that may be administered in conjunction with DI particle-rich viral populations include, for example, live attenuated virus, dead virus, non-viable cells (e.g., killed tumor cells expressing at least one tumor cell antigen), and tumor homogenates. Details pertaining to therapeutic approaches involving a combination of DI particle-rich viral populations of the invention and conventional vaccine strategies are described in detail herein below.

The present invention encompasses a method for stimulating an immune response in a subject, comprising administering to a subject at least one antigen, wherein the at least one antigen is administered in conjunction with a defective interfering (DI) particle-enriched viral population, and wherein the at least one antigen and the DI particle-enriched viral population are administered in an effective amount capable of inducing an antigen specific immune response in said subject.

With respect to effective dose ranges for administering DI particle-enriched viral populations in conjunction with either an isolated antigen or a conventional vaccine, a range of 1,000 to 20,000 infectious particles is envisioned. In a particular embodiment, a range of 5,000 to 15,000 infectious particles is envisioned. In a particular embodiment, 10,000 infectious particles is envisioned. It is anticipated that at least 5 times more DIs than standard virus would be an effective amount with which to supplement a vaccine. One of ordinary skill in the art would appreciate that the ratio of DI to standard virus may be varied in accordance with the particular combination of DI particles and virus. A ratio of 5:1 with respect to DI particles and standard virus may, therefore, be viewed as a starting point from which variation in the ratio may be introduced for the purposes of optimization.

In another embodiment, the present invention is directed to a method for stimulating an enhanced immune response in a subject, the method comprising administering to a subject (a) a virus vaccine (e.g., a live attenuated virus vaccine); and (b) a plurality of DI particles complementary to the virus or a plurality of recombinant DI particles complementary to the virus, wherein the virus vaccine and the DI particles are administered in an effective amount capable of eliciting an enhanced immune response to the virus in the subject. The administration of a viral vaccine and a plurality of complementary DI particles or complementary recombinant DI particles may be performed in any order, and may be administered separately in either order or concomitantly. As indicated herein, the term complementary is used to refer to viruses and DI particles or recombinant DI particles that are compatible with respect to providing the machinery necessary for DI particle replication. The DI particles or recombinant DI particles of such complementary combinations may be either homologous or heterologous with respect to the virus. In accordance with the discovery of the present inventors, administration of a conventional virus vaccine supplemented with complementary DI particles or complementary recombinant DI particles serves to elicit an enhanced immune response which is more robust than that stimulated by administration of the virus vaccine alone. The present inventors have demonstrated that enhanced immune responses so generated in mouse models, for example, are 15-20 times more robust with respect to a viral response and the antibody response is at least 10 times better in mouse models.

The present invention further includes a method for enhancing an immune response to an antigen, wherein a recombinant virus (e.g., a recombinant paramyxovirus or orthomyxovirus) capable of generating DI particles is engineered to encode an antigen of interest and is subsequently passaged to generate a population of recombinant viruses and DI particles derived therefrom, and administration of this DI particle-rich viral population to a subject promotes dendritic cell maturation and thus enhances the subject's immune response to the encoded antigen. A DI particle-rich viral population so generated is a homologous DI particle-rich viral population. An exemplary homologous DI particle-rich viral population comprises, e.g., a recombinant Sendai virus encoding an antigen of interest and homologous (Sendai virus) DI particles. As described herein, Sendai virus is an exemplary paramyxovirus of the invention because it produces DI particles and has been shown to be safe for use in humans. See, e.g., Slobod et al. Vaccine 22:3182-3186 (2004).

Recombinant viruses (e.g., paramyxoviruses and orthomyxoviruses) and recombinant DI particles derived therefrom and compositions thereof are also encompassed by the present invention. DI particle-rich viral populations derived from, e.g., recombinant paramyxoviruses and orthomyxoviruses of the invention and compositions thereof are also included in the present invention.

The invention also encompasses a method for enhancing an immune response to an antigen, wherein a recombinant virus (e.g., a recombinant paramyxovirus or orthomyxovirus) capable of generating DI particles is engineered to encode an antigen of interest and mixed with homologous (of the same virus strain) and/or heterologous (of a different virus or virus strain) complementary DI particles and administration of the resultant DI particle-rich viral population to a subject promotes dendritic cell maturation and thus enhances the subject's immune response to the encoded antigen. A DI particle-rich viral population so generated may be either a homologous DI particle-rich viral population or a complementary heterologous DI particle-rich viral population. An exemplary homologous DI particle-rich viral population may comprise a recombinant Sendai Cantell virus encoding an antigen of interest and homologous (Sendai Cantell virus) DI particles, whereas an exemplary heterologous DI particle-rich viral population may comprise a recombinant Sendai Cantell virus encoding an antigen of interest and heterologous (non-Sendai virus or Sendai 52) complementary DI particles.

The present invention further encompasses heterologous DI particle-rich viral populations comprising mixtures of a first standard virus population and DI particles isolated from a second standard virus population or genetically engineered based on the genomic composition of a second complementary standard virus. In an aspect of the invention, a heterologous DI particle-rich viral population comprises a first standard paramyxovirus population and DI particles isolated from a second complementary standard paramyxovirus population. Paramyxoviruses of utility in the compositions and methods of the present invention include, but are not limited to: Parainfluenzavirus 1-4, Sendai virus, Bovine Parainfluenzavirus type 3, Simian virus 5, Mumps virus, Newcastle disease virus, Measles virus, Canine Distemper Virus, Hendra virus, Nipha virus, Equine morbillivirus, Respiratory Syncytial Virus, Bovine Respiratory Syncytial Virus, and Avian pneumovirus. Orthomyxoviruses are also useful in the compositions and methods of the present invention. Additional viruses from the families shown in Table I are also envisioned as useful in the invention.

With respect to complementation among viruses, different strains of influenza virus (influenza A and B) have been shown to complement each other (Jambrina et al. Virology, 1997 235:209). Data presented herein reveal that Sendai Cantell DI particles can be complemented by Sendai 52. Some strains of West Nile virus have also been shown to replicate DIs from other strains (Debnath et al. J Gen Virol, 1991 72:2705). With respect to different togaviruses, Sindbis virus and Semliki Forest virus are known to complement each other (Barrett et al. J Gen Virol, 1984, 65:1119). In that all paramyxoviruses have very similar requirements for replication, and high homology between crucial elements in their promoters, it is likely that different paramyxoviruses will generally complement each other. A critical component for heterologous complementation appears to be the relatedness of the polymerases of the different viruses being investigated. In other words, polymerase compatibility is an important component that contributes to the ability of different viruses to cross-complement heterologous DI particles.

Compositions comprising heterologous DI particle-rich viral populations of the invention are also envisioned.

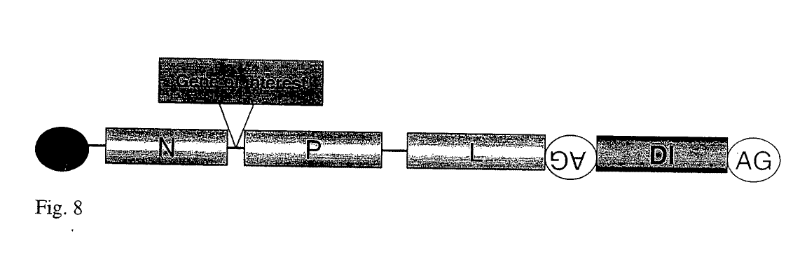

In another embodiment of the invention, a recombinant defective Sendai virus construct is envisioned. As shown in FIG. 8, a recombinant defective Sendai virus construct includes a virus genome, which is flanked by the normal genomic (G) and antigenomic (AG) promoters, and minimally comprises the genes necessary for replication (NP, P, and L) and a nucleic acid sequence encoding a polypeptide of interest. The absence of packaging proteins renders such viral constructs defective in packaging and spreading. Recombinant viruses encoded by such constructs are propagated in a cell line that expresses the additional proteins necessary for packaging a viral genome. In that these viruses code for a DI particle flanked by the AG promoter and a complementary copy of this promoter, the genomes are efficiently replicated and thus produce dsRNA, which in turn mediates the effects of DI particle enhancement of immunity. Experimental evidence generated by the present inventors suggests that the dsRNA effective for the enhancement of DC maturation comes from viral genome replication and is independent of sense strand synthesis. Viral genome replication is, therefore, believed to be the process whereby dsRNA is produced. The secondary structure of the DIs (as a panhandle) provides another possible source of dsRNA.

The invention also presents recombinant packaging defective paramyxoviruses or orthomyxoviruses comprising a complementary antigenomic promoter. With respect to the position of the additional complementary antigenomic promoter, in general, the shorter the DI the more efficient it is anticipated to be. For Sendai virus, for example, whose full length DI is about 1400 nucleotides, a shorter antigenomic promoter bearing DI is envisioned to be about 600-700 nucleotides. In other words, the inverted antigenomic promoter is positioned at this distance relative to the 5′ end of the Sendai genome. It is also envisioned that a particular sequence may be identified that is responsible for the production of DI particles. Such a sequence could be used instead of an inverted antigenomic promoter to produce DIs. If such a sequence is present in the genome and acts as the “signal” for the formation of the DIs, such “genetic-DI-forming signal sequences” could be added to recombinant viruses to promote a higher rate of DI-production.

In an aspect of the invention, a recombinant defective Sendai virus construct of the invention is transfected into DCs isolated from a patient (or expanded in vitro following isolation from the patient's blood or bone marrow) so as to induce DC maturation. This approach reduces complications that could potentially arise from immune reactions generated against viral coat proteins (packaging proteins), such as the generation of neutralizing antibodies. As such, a transfectable recombinant defective Sendai virus construct could be used for multiple immunizations.

Recombinant defective paramyxoviruses and orthomyxoviruses, recombinant DI particles derived therefrom, and compositions thereof are also included in the present invention, as are methods of using same.

In another embodiment, the invention encompasses genetically engineered Sendai virus DI particles that comprise a nucleic acid sequence encoding a polypeptide of interest. Such genetically engineered Sendai virus DI particles may be isolated from a recombinant Sendai virus or generated de novo from a construct.

With respect to recombinant Sendai virus, which is replicative, such a virus comprises all of the necessary proteins that enable it to multiply in vivo, and further includes a cloning site for a protein of interest Recombinant Sendai virus may also be engineered to produce “more” DIs by inserting either a complementary inverted antigenomic promoter at an appropriate position or a DI-“making sequence”.

Alternatively, such a recombinant Sendai virus may also be passaged in such a manner so as to promote DI particle production and DI particles so generated are isolated as described herein.

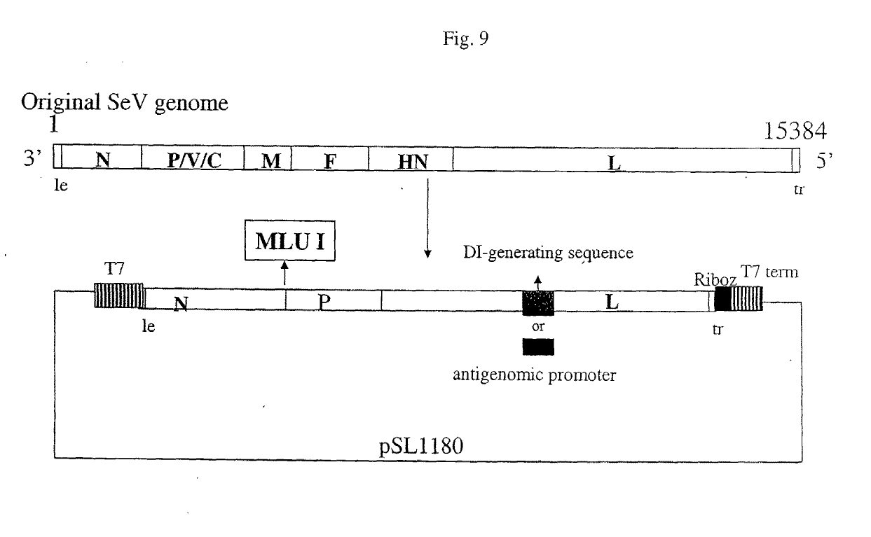

Alternatively, it is possible to use reverse genetics to generate plasmids capable of producing large populations of DI particles, wherein such plasmids comprise DI-particle generating sequences (see FIG. 9). When co-infected with a complementary virus into a cell or supplemented in vitro by providing the proteins necessary to generate viral particles, such plasmids comprising DI-particle generating sequences can be used as a source of DI particles. The DI-particle producing plasmids may also comprise a cloning site (e.g., Mlu I) into which a nucleic acid sequence encoding a protein of interest may be inserted, so as to facilitate expression of the protein. Alternatively, a plasmid may comprise a complementary antigenomic promoter which would enable generation of DI particles upon genome replication.

Compositions comprising genetically engineered Sendai virus DI particles and methods of using same are also encompassed by the present invention.

Also presented is a method for activating a dendritic cell, comprising contacting a dendritic cell with at least one antigen in conjunction with a DI particle-enriched viral population, in an amount effective to activate the dendritic cell, wherein the method is performed ex vivo. The method may further comprise administering the ex vivo activated dendritic cell to a subject as described herein below.

In one aspect, the invention encompasses a method comprising isolating DCs from a subject and treating these DCs ex vivo with a DI particle-enriched viral population (e.g., a DI particle-enriched Sendai virus population) in combination with an antigen of interest and re-introducing these activated, mature DCs back into the subject. Ex vivo DCs may be obtained directly from blood or other tissue and purified using standard techniques or grown from blood monocytes or bone marrow in the presence of appropriate growth factors. Once the cells are isolated they can be infected with the DI particle-enriched viral population. Recombinant DI particle-enriched viral populations that are engineered to express the antigen of interest may also be used in this aspect of the invention. Re-introduction of such matured DCs confers upon the subject an enhanced ability to mount an immune response against the antigen of interest. Antigens of utility for such purposes are described in detail herein and include, without limitation, antigens isolatable from an infectious agent, tumor cell antigens, and allergens.

Experimental data generated in mouse models reveal that a significant immune response is induced upon administration of 100,000 activated (matured) DCs. For humans, approximately 1,000,000 activated, matured DCs may be administered to achieve a significant immune response. With respect to antigen, at least one standard virus particle plus complementary DIs (approximately a 5:1 ratio of DI particles to standard virus) per cell may be used to advantage to infect isolated DCs. The above indicated guidelines also apply to applications wherein defective packaging viruses are used. Where appropriate, methods for transfecting a viral construct may also be used to mature a DC. Transfecting means such as those known in the art, including electroporation, and calcium phosphate or liposomal mediated transfection are, therefore, envisioned as alternatives to infecting DCs with viral constructs or particles.

Applications Directed to Cancer Therapy

Recombinant Sendai virus: Recombinant virus capable of making DI particles (e.g., Sendai virus) expressing a tumor-specific protein can be engineered. Such tumor-specific antigens include, but are not limited to Carcinoembryonic antigen (CEA); Alpha-fetoprotein; Alkaline phosphatase isoenzyme; Sialyl tn; MART-1/melan-A; NY-ESO-1; P53; KLF6; Tyrosinase; RAS; and oncogene products in general. Other oncogene products of utility in the present invention are known to skilled practitioners. Recombinant virus can then be grown in conditions that encourage the production of DI particles. Such conditions are described herein and known in the art and include: multiple passages of undiluted recombinant virus and supplementation with compatible or complementary DI particles. DI particle-rich virus preparations can then be administered to a subject to induce a potent immune response against the tumor antigen, and thus confer to the subject an enhanced ability to target cancer cells expressing the tumor cell antigen for lysis by cytotoxic T-cells.

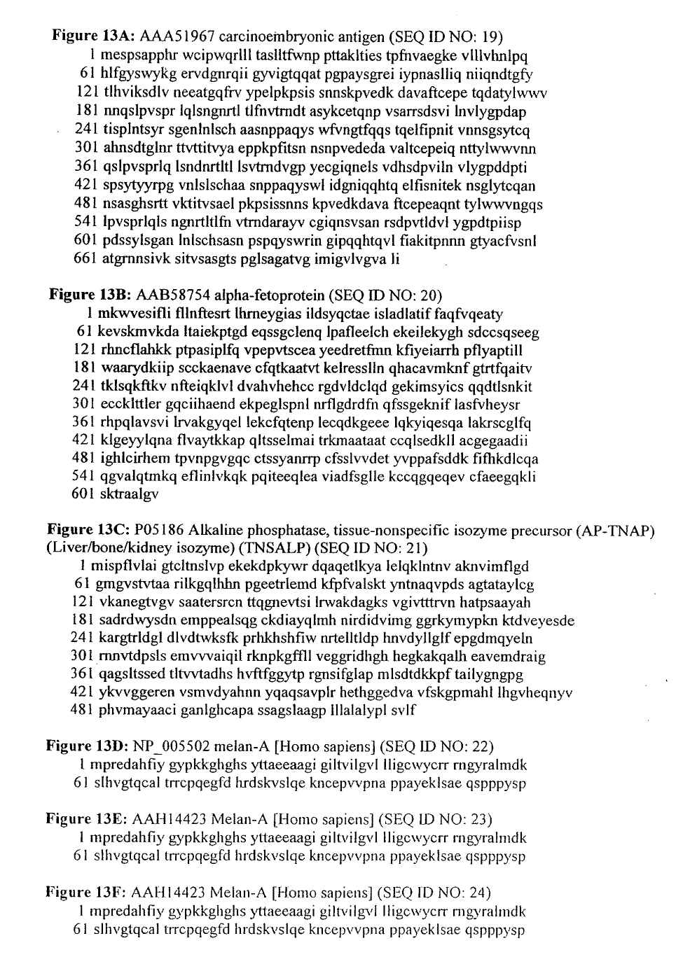

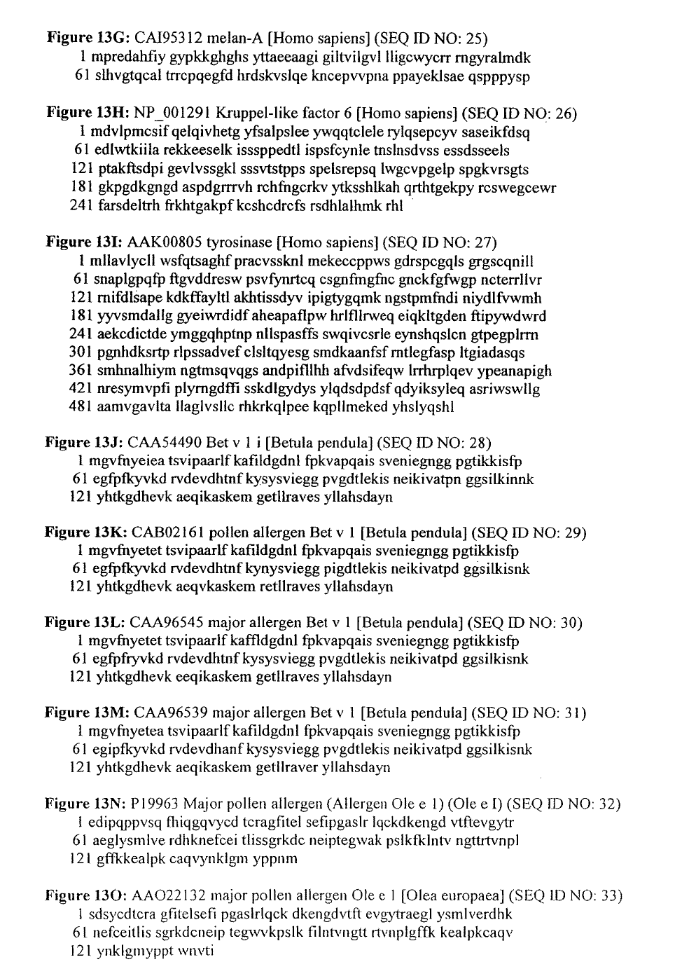

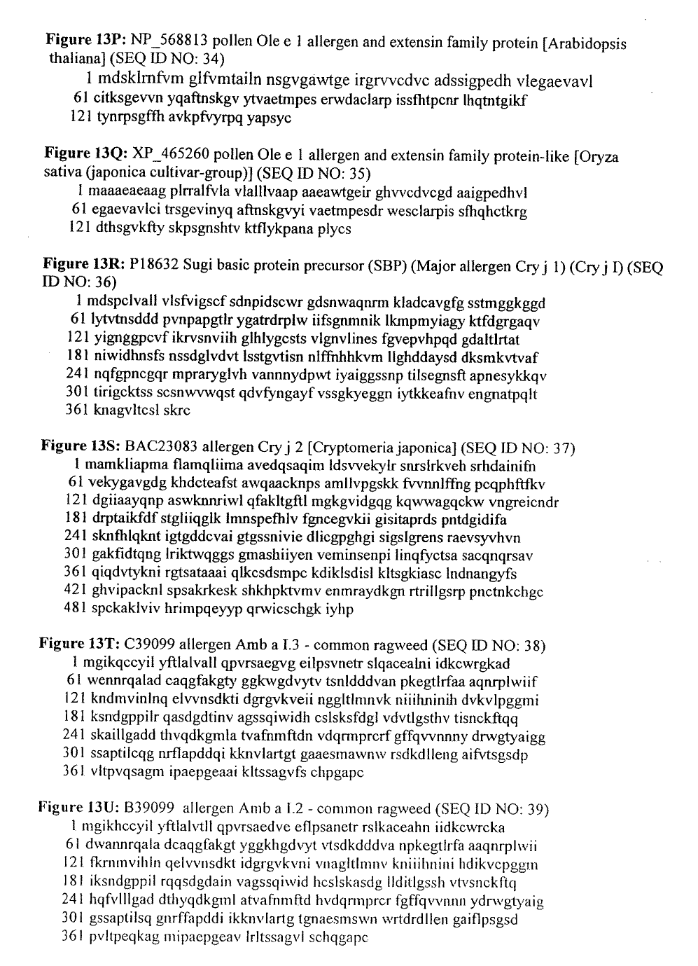

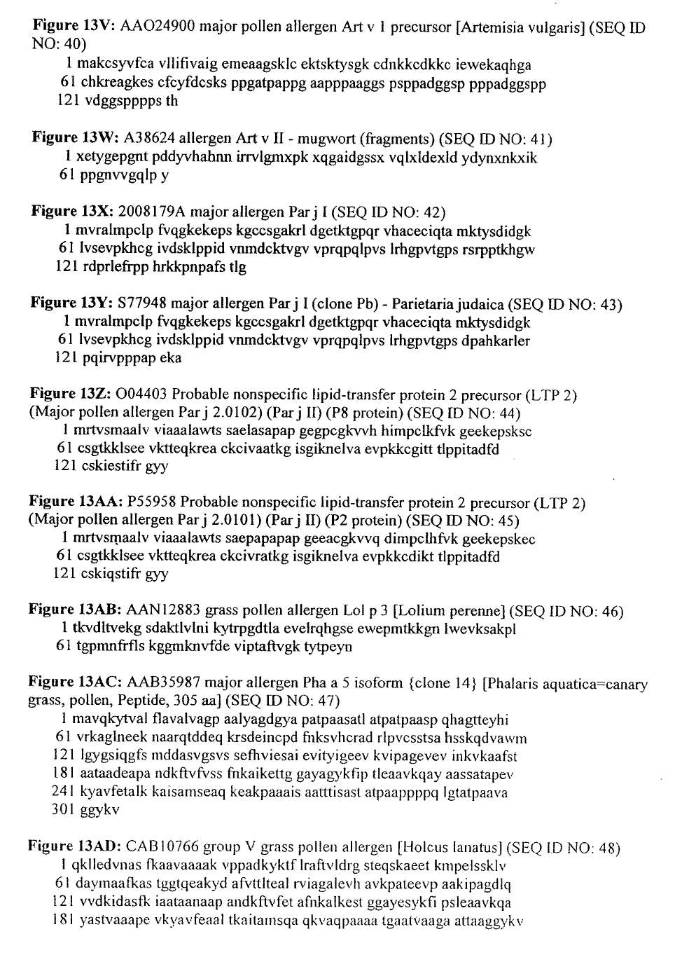

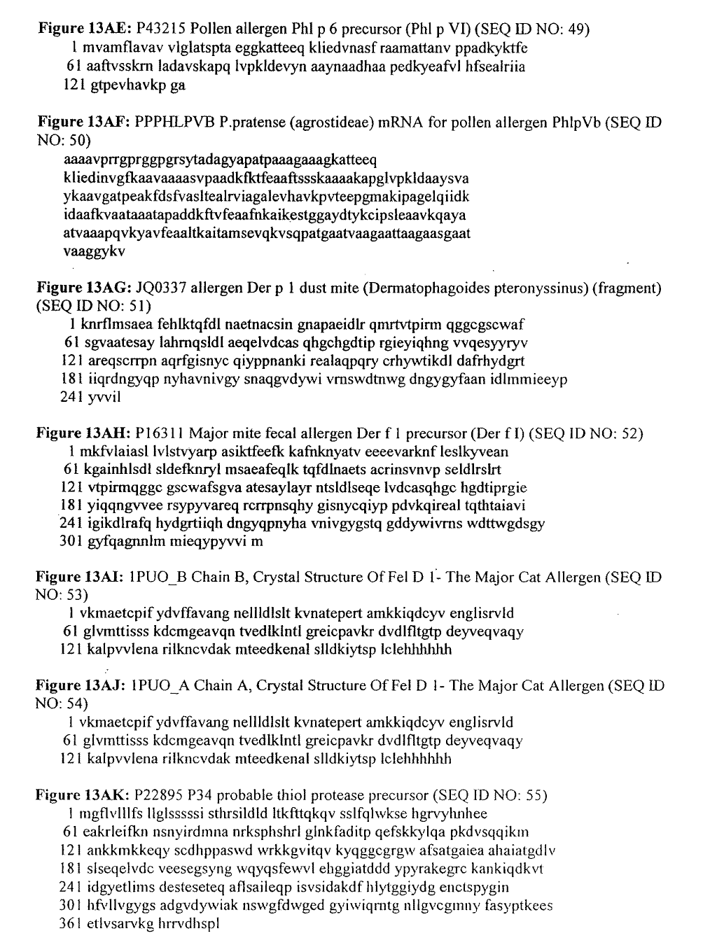

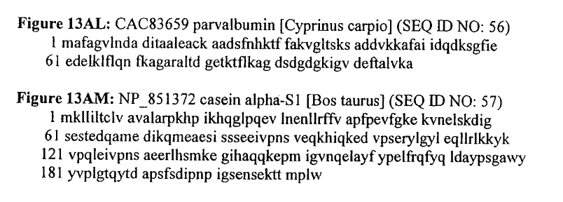

The following list of GenBank Accession numbers presents amino acid sequences corresponding to exemplary tumor antigens which may used in the context of the present invention. Exemplary such tumor antigens include, but are not limited to: CEA: AAA51967; ALPHA-FETOPROTEIN: AAB58754; ALKALINE PHOSPHATASE ISOENZYM: P05186; MART-1/MELAN-A: NP_{—}005502, AAH14423, A55253, CAI95312; KRUPPEL-LIKE FACTOR-6 (KLF-6): NP_{—}001291; and TYROSINASE: AAK00805. See FIG. 13 for sequences.

Packaging defective recombinant Sendai virus: Packaging defective recombinant viruses are also encompassed by the present invention. A packaging defective recombinant Sendai virus construct, for example, is an exemplary recombinant viral construct of the invention. See FIG. 8. As shown therein, the virus genome is flanked by the normal genomic (G) and antigenomic (AG) promoters. This virus would minimally comprise the genes necessary for replication (NP, P, and L) and a tumor-specific gene of interest. The absence of packaging proteins renders such viruses defective in packaging and spreading. Recombinant viruses encoded by such constructs are propagated in a cell line that expresses the additional proteins necessary for packaging the viral genome. In that these viruses code for a DI particle flanked by the AG promoter and a complementary copy of this promoter, the genomes are efficiently replicated and thus provide the dsRNA thought to mediate the effects of DI particle enhancement of immunity. In view of these features, these recombinant viruses are potent inducers of dendritic cell maturation and immunity.

Defective recombinant virus may also be used in conjunction with anti-cancer agents to augment cancerous cell death by creating a more immunogenic environment in a subject afflicted with cancer. Such recombinant DI particles may be engineered to encode a chemotherapeutic. Alternatively or in addition, DI particle enriched populations or recombinant DI particles may be administered in conjunction with anti-cancer agents. Such combination therapy enhances cancer cell death and promotes a robust immune response capable of killing any residual cancer cells that have escaped treatment. [Lake et al. New Engl J of Med 354, 2503 (2006)].

Alternatively, DCs isolated from a subject can be transfected ex vivo with packaging defective recombinant viral constructs, such as the exemplary packaging defective recombinant Sendai virus construct. Accordingly, administration of such activated DCs can be performed multiple times without encountering complications associated with an immune response generated against viral packaging proteins. Alternatively, DCs may be transfected in vivo with the packaging defective recombinant viral constructs of the invention. Transfection protocols for ex vivo and in vivo applications are described herein and known to ordinarily skilled practitioners.

Induction of cross priming against tumor cells: Cross priming is the process by which dendritic cells phagocytose other cells and present the proteins from these cells in association with MHC class I. Under normal circumstances, a DC is constantly processing dying cells within the body and presenting antigens thereof. In general, however, this does not result in an immune response since these dendritic cells tend to be in an immature state. Recent findings reveal that a more effective immune response is generated through the cross priming pathway when the phagocytosed cell is infected with virus or contains dsRNA. This is most likely due to maturation of the DC via dsRNA mediated activation of TLR3. The present invention presents methods to promote such DC maturation for the purposes of cancer therapy. In brief, cancer cells (e.g., tumor cells) are isolated from a patient, lethally irradiated (e.g., the irradiation dose for human tumor cells is 20,000 rad), infected with DI particle-rich Sendai virus or a replication defective construct as described above and injected back into the patient. The DI particles provide a strong source of dsRNA which would activate the immune system against the cancer cell or tumor through the cross priming pathway.

Alternatively, cancer cells (e.g., tumor cells) isolated from a patient can be infected with DI particle-rich virus population (e.g., a DI particle-rich Sendai virus population) or a packaging defective construct as described above and used to treat DCs ex vivo. The mature ex vivo treated DCs are then re-introduced into the patient wherein they promote a robust immune response directed against the cancer cells.

Applications Directed to Allergen Desensitization

Subjects afflicted with allergy have an exaggerated immune response to allergens, which is characterized by the production of allergen-specific antibodies of the IgE type. T cells from allergic patients respond to particular allergens by inducing expression of cytokines typically produced by T helper 2 (Th2) cells [interleukin-4, -5, and -6 (IL-4, IL-5, and IL-13)], rather than cytokines produced by T helper 1 (Th1) cells [interferon gamma (IFN-gamma) and IL-2]. Th2 cell cytokines stimulate the production of IgEs, whereas Th1 cytokines bias towards IgG subtype production. It is, therefore, desirable to bias anti-allergen immunity towards a Th1 type so as to modulate the allergic response. In this scenario, an allergen is more likely to encounter and bind to IgG antibodies, rather than IgE antibodies, upon entering the body. Thus, IgE-mediated responses such as release of vasoactive substances from mast cells are ameliorated or abrogated.

A DI-rich viral preparation mixed with the allergen or a construct expressing the allergen of interest and able to make hi DIs, could be used to bias immunity against the allergen toward a Th1 type. The present inventors and others have shown that the nature of the stimuli for DC maturation determines the kind of T helper immune response. Notably, DI-mediated maturation of DCs promotes the generation of Th1-inducers. Secretion of IL-12 is, for example, a hallmark for Th1-inducing DCs. See also Kay. N Engl J. Med. 2001, 344(2):109-13; Jenmalm et al. Clin Exp Allergy. 2001, 31(10):1528-35; Stokes et al. Ann Allergy Asthma Immunol. 2004, 93(3):212-7; quiz 217-9, 271 for additional commentary.

The following list of GenBank Accession numbers presents amino acid sequences corresponding to exemplary allergens which may used in the context of the present invention. Exemplary such allergens include, but are not limited to: tree pollen major allergens: Bet v 1: CAA54490, CAB02161, CAA96545, CAA96539; Ole e 1: P19963, AA022132, NP_{—}568813, XP_{—}465260, Cry j 1: P18632, and Cry j 2: BAC23083; ragweed: Amb a 13: C39099 and Amb a 12: B39099; mugwort: Art v 1: AA024900, A38624; pellitory: Par j 1: 2008179A, S77948, and Par j 2: 004403, P55958; grass pollen: Lol p 3: AANI2883, Pha a 5: AAB35987, group V: CAB 10766, Phlp 6: P43215, PhlpVb: PPPHLPVB; dust mite allergens: Der p 1: IQ0337 and Der f 1: P16311; cat allergens: Fed d 1: IPUOB, IPUOA; soybean major allergen: P34: P22895; fish: parvalbumins: CAC83659; and milk: caseins: NP_{—}851372. See FIG. 13 for sequences.

Recombinant Sendai vines: Recombinant Sendai virus expressing an allergen can also be engineered. Such allergens include, but are not limited to allergenic polypeptides such as those that trigger allergic reactions to toxins, drugs (e.g., antibiotics and serums), foods (e.g., milk, chocolate, strawberries, wheat, and nuts), infectious bacteria; viruses, animal parasites, and inhalants (e.g., dust, pollen, perfumes, and smoke, animal allergens,). Additional allergens of utility in the present invention are known to skilled practitioners. Such recombinant virus can be grown in conditions that encourage the production of DI particles. Such conditions are described herein and known in the art and include multiple passages of undiluted recombinant virus. DI particle-rich virus preparations can then be administered to a subject to modulate their immune response to the allergen to promote a predominantly Th1-driven response.

Packaging defective recombinant Sendai virus: Replication defective recombinant viruses are also encompassed by the present invention. Replication defective recombinant Sendai virus, for example, is an exemplary recombinant virus of the invention. See FIG. 8. As shown therein, the virus genome is flanked by the normal genomic (G) and antigenomic (AG) promoters. This virus would minimally comprise the genes necessary for replication (NP, P, and L) and a gene encoding an allergen of interest. The absence of packaging proteins renders such viruses defective in packaging and spreading. Recombinant viruses are propagated in a cell line that expresses the additional proteins necessary for packaging the viral genome. In that these viruses code for a DI particle flanked by the AG promoter and a complementary copy of this promoter, the genomes are efficiently replicated in both positive and negative RNA sense and thus provide the dsRNA thought to mediate the effects of DI particle enhancement of immunity. In view of these features, these recombinant viruses are potent inducers of dendritic cell maturation and immunity.

Combined Use of DI Particle-rich Viral Populations and Conventional Vaccine Strategies The present invention encompasses the use of mixtures of DI particles and standard virus capable of complementing these DI particles functionally (DI particle-rich viral populations) in conjunction with various active vaccination protocols well known in the art. In contrast to passive vaccination, active vaccination is intended to stimulate a subject's immune system to mount a humoral and/or cellular immune response to, for example, an antigen or a pathogen. Such DI particle-rich viral populations may be homologous or heterologous mixtures of DI particles and standard virus. Moreover, it is envisioned that DI particle-rich viral populations may be added separately, but in conjunction with conventional vaccine formulations, or mixed with conventional vaccine formulations. Such combined vaccination strategies are designed to enhance or augment immune responses elicited by the “conventional vaccine” by enhancing DC maturation. Conventional vaccine strategies that may be used advantageously in combination with DI particle-rich viral populations include, but are not limited to: subunit vaccines; recombinant live viral and bacterial vaccine-delivery vectors; nucleic acid vaccines; virus-like particles (VLPs); modified virus vaccines; inactivated virus vaccines; and live attenuated virus vaccines. Such conventional vaccine strategies are known in the art and described briefly herein below. Conventional vaccines that may be used as components of combination therapy with DI particle-rich viral populations include without limitation: Hepatitis B, streptococcus pneumoniae, neisseria meningitidis, Hemophilus influenzae, pertussis toxin, tetanus toxin, and inactivated virus vaccines.

Presently available viral vaccines (conventional viral vaccines) include killed or attenuated live viral vaccines, live-vectored vaccines, subunit vaccines, and DNA or RNA vaccines. See Roth et al., “New Technology For Improved Vaccine Safety And Efficacy”, Veterinary Clinics North America: Food Animal Practice 17(3): 585-597 (2001). Attenuation of viruses can be achieved by UV irradiation, chemical treatment, or high serial passage in vitro. The number, position and nature of mutations induced by these methods are unknown absent genomic sequence analyses. Attenuation can also be achieved by making defined genetic alterations, for example, specific deletion of viral sequences known to confer virulence, or insertion of sequences into the viral genome.

Live, attenuated virus vaccines mimic natural exposure while avoiding disease, with the expectation that immunologic memory and lifelong immunity will be induced. Such vaccines effectively induce both humoral and cell-mediated immunity, and generally require only one or two immunizations, since the immune responses they induce are very durable. Most licensed vaccines in use today are based on this concept. Exemplary vaccines comprising live, attenuated viruses include, for example, vaccines for the measles, mumps, and rubella (“German measles”), chicken pox (Varicella), yellow fever, small pox, and the Sabin oral polio vaccine (OPV).

Genetic Vaccines

Subunit vaccines. Subunit vaccines, which generally consist of one or more isolated proteins, peptides, or polysaccharides derived from a pathogen, may be used to advantage to confer immunity to infection by the pathogen. These isolated proteins, peptides, or polysaccharides act as target antigens against which an immune response may be mounted. The proteins, peptides, or polysaccharides selected for a subunit vaccine are normally displayed on the cell surface of the pathogen, such that when the subject's immune system is subsequently challenged by the pathogen, it recognizes and mounts an immune reaction to the cell surface proteins, peptides, or polysaccharides and, by extension, the attached pathogen. Because subunit vaccines are not whole infective agents, they are incapable of becoming infective. Thus, they present no risk of undesirable virulent infectivity, a significant drawback associated with other types of vaccines. Moreover, it has been reported that recombinant subunit vaccines such as the hepatitis B surface antigen vaccine (HBsAg) stimulate a more specific protective T helper and humoral immune response against a single antigen. See also Hansson et al. Biotechnol. Appl. Biochem. 32:95-107 (2000).

Absent an active infection, however, a complete immune response may not be elicited. Subunit vaccines, which comprise discrete components of a pathogen, do not undergo an infective cycle and often do not elicit the CTL arm of the cellular immune response. Absent the CTL arm, the immune response elicited by a subunit vaccine may be insufficient to adequately protect an individual. In addition, subunit vaccines have the additional drawback of being both expensive to produce and purify. Such considerations are known to skilled practitioners and would, accordingly, be considered with respect to aspects of the present invention directed to the use of subunit vaccines.

Accordingly, the most effective vaccines for invoking a strong and complete immune response carry the most risk for harming the individual, while the safer alternatives may induce an incomplete, and therefore, less effective immune response. Furthermore, many subunit vaccines and recombinant vaccines using non-virulent vectors to produce target proteins are most useful if a single antigenic component can be identified which is singularly protective against live challenge by a pathogen. Both technologies, therefore, work optimally when such a protective component(s) has been identified, but identification of such targets is often both laborious and time-consuming.

Nucleic Acid Vaccines. The direct introduction of a normal, functional gene into a living animal has been studied as a means for replacing defective genetic information. In such studies, DNA is introduced directly into cells of a living animal. The following references pertain to methods for the direct introduction of nucleic acid sequences into a living animal: Nabel et al., (1990) Science 249:1285-1288; Wolfe et al., (1990) Science 247:1465-1468; Acsadi et al. (1991) Nature 352:815-818; Wolfe et al. (1991) BioTechniques 11(4):474-485; and Felgner and Rhodes, (1991) Nature 349:351-352.

For some applications, the present invention relates to the use of genetic material (e.g., nucleic acid sequences) as immunizing agents. In one aspect, the present invention relates to the introduction of exogenous or foreign DNA or RNA molecules into a subject's tissues or cells, wherein these molecules encode an exogenous protein capable of eliciting an immune response to the protein. See, for example, Hansson et al. Biotechnol. Appl. Biochem. 32:95-107 (2000). The exogenous nucleic acid sequences may be introduced alone or in the context of an expression vector wherein the sequences are operably linked to promoters and/or enhancers capable of regulating the expression of the encoded proteins. The introduction of exogenous nucleic acid sequences may be performed in the presence of a cell stimulating agent capable of enhancing the uptake or incorporation of the nucleic acid sequences into a cell. Such exogenous nucleic acid sequences may be administered in a composition comprising a biologically compatible or pharmaceutically acceptable carrier. The exogenous nucleic acid sequences may be administered by a variety of means, as described herein, and well known in the art.

Such methods may be used to elicit immunity to a pathogen, absent the risk of infecting an individual with the pathogen. The present invention may be practiced using procedures known in the art, such as those described in PCT International Application Number PCT/US90/01515, wherein methods for immunizing an individual against pathogen infection by directly injecting polynucleotides into the subject's cells in a single step procedure are presented.

In one aspect, the present invention relates to methods for eliciting immune responses in an individual or subject which can protect the individual from pathogen infection. Accordingly, genetic material that encodes an immunogenic protein is introduced into a subject's cells either in vivo or ex vivo. The genetic material is expressed by these cells, thereby producing immunogenic target proteins capable of eliciting an immune response. The resulting immune response is broad based and involves activation of the humoral immune response and both arms of the cellular immune response.

This approach is useful for eliciting a broad range of immune responses against a target protein. Target proteins may be proteins specifically associated with pathogens or the individual's own “abnormal” or infected cells. Such an approach may be used advantageously to immunize a subject against pathogenic agents and organisms such that an immune response against a pathogen protein provides protective immunity against the pathogen. This approach is particularly useful for protecting an individual against infection by non-encapsulated intracellular pathogens, such as a virus, which produce proteins within the host cells. The immune response generated against such proteins is capable of eliminating infected cells with CTLs.

The immune response elicited by a target protein produced by vaccinated cells in a subject is broad-based and includes B and T cell activation, including cytotoxic T cell (CTL) activation. In brief, target antigen produced within the cells of the host is processed intracellularly into small peptides, which are bound by Class I MHC molecules and presented in the context of cell surface Class I. The Class I MHC-target antigen complexes are capable of stimulating CD8⁺ T cells, which are predominantly CTLs. This property of genetic immunization, namely the ability to elicit CTL responses (killer cell responses), renders approaches involving genetic immunization particularly effective.

The CTL response is crucial for conferring protection against pathogens such as viruses and other intracellular pathogens which produce proteins within infected cells. Similarly, the CTL response can be utilized for the specific elimination of deleterious cell types which, during their production of proteins, display antigens bound by Class I MHC molecules. Therefore, genetic immunization may be more likely to result in anti-pathogen protection and clinical improvement in patients than standard immunization methods directed to use of killed, inactivated or protein- or peptide-based subunit vaccines.

Genetic vaccines may be administered to cells in conjunction with compounds that stimulate cell division and facilitate uptake of genetic constructs. This step provides an improved method of direct uptake of genetic material. Administration of cell stimulating compounds results in a more effective immune response against the target protein encoded by the genetic construct.

According to the present invention, DNA or RNA that encodes a target protein is introduced into the cells of an individual wherein it is expressed, thus producing the target protein. The DNA or RNA is linked to regulatory elements necessary for expression in the cells of the subject. Regulatory elements may include a promoter and a polyadenylation signal. Other elements known to skilled artisans may also be included in genetic constructs of the invention, depending on the application.

As used herein, the term “genetic construct” refers to a DNA or RNA molecule that comprises a nucleic acid sequence which encodes a target protein and which includes initiation and termination signals operably linked to regulatory elements including a promoter and polyadenylation signal capable of directing expression in the cells of a vaccinated individual. As used herein, the term “expressible form” refers to gene constructs which contain the necessary regulatory elements operably linked to a coding sequence of a target protein, such that when present in the cell of the individual, the coding sequence is expressed. As used herein, the term “genetic vaccine” refers to a pharmaceutical preparation that comprises a genetic construct.

The present invention provides genetic vaccines, which include genetic constructs comprising DNA or RNA which encodes a target protein. As used herein, the term “target protein” refers to a protein capable of eliciting an immune response. The target protein is an immunogenic protein derived from the pathogen or undesirable cell-type such as an infected or transformed cell. In accordance with the invention, target proteins may be pathogen-associated proteins, tumor associated proteins, or allergens. The immune response directed against the target protein protects the individual against the specific infection or disease with which the target protein is associated. For example, a genetic vaccine comprising a DNA or RNA molecule that encodes a pathogen-associated target protein is used to elicit an immune response that will protect the individual from infection by the pathogen.

A genetic construct may comprise a nucleotide sequence that encodes a target protein operably linked to regulatory elements needed for gene expression. Accordingly, incorporation of the DNA or RNA molecule into a living cell results in the expression of the DNA or RNA encoding the target protein and thus, production of the target protein.

Following introduction into a cell, a genetic construct comprising a nucleic acid sequence encoding a target protein operably linked to the regulatory elements may be maintained episomally or may be integrated into the cell's chromosomal DNA. DNA may be introduced into cells as a plasmid or as linearized DNA. When introducing DNA into a cell, reagents which promote DNA integration into chromosomes may be added. DNA sequences capable of promoting integration may also be included in the DNA molecule. Since integration into the chromosomal DNA necessarily requires manipulation of the chromosome, maintaining the DNA construct as an episome is generally preferred. This reduces the risk of damaging the recipient cell by chromosomal insertion and does not adversely alter the effectiveness of the vaccine. Alternatively, RNA may be administered to the cell and DI particle supplementation would serve as an adjuvant only in this context.

The necessary elements of a genetic construct include a nucleotide sequence that encodes a target protein and the regulatory elements necessary for expression of that sequence in the cells of the vaccinated individual. The regulatory elements are operably linked to the DNA sequence that encodes the target protein to enable expression. The nucleotide sequence that encodes the target protein may be cDNA, genomic DNA, synthesized DNA or a hybrid thereof or an RNA molecule such as mRNA. Accordingly, as used herein, the terms “DNA construct”, “genetic construct” and “nucleotide sequence” may refer to constructs comprising DNA or RNA.

The regulatory elements necessary for gene expression include, but are not limited to, a promoter, an initiation codon, a stop codon, and a polyadenylation signal. These elements must be operable in the vaccinated individual and be operably linked to the nucleotide sequence that encodes the target protein in such a way as to facilitate expression of the target protein in cells of a vaccinated individual.

Initiation codons and stop codons are generally components of the nucleotide sequence encoding the target protein. Such sequences may alternatively be derived from different nucleic acid sources so as to optimize functionality and expression of the target protein in cells of a vaccinated individual. Similarly, promoters and polyadenylation signals used must be functional within the cells of the vaccinated individual.

Examples of promoters useful for practicing this aspect of the present invention, (especially for a genetic vaccine intended for use in humans), include, but are not limited to the Mouse Mammary Tumor Virus (MMTV) promoter, Human Immunodeficiency Virus Long Terminal Repeat (HIV LTR) promoter, Moloney virus promoter, Cytomegalovirus (CMV) promoter, human actin promoter, human myosin promoter, Rous sarcoma virus (RSV) promoter, human hemoglobin promoter, human muscle creatine promoter, and Epstein Barr virus (EBV) promoter.

In order to be a functional genetic construct, the regulatory elements must be operably linked to the nucleotide sequence that encodes the target protein. Accordingly, it is necessary for the promoter and polyadenylation signal to be in frame with the coding sequence. In order to maximize protein production, regulatory sequences may be selected which are well suited for gene expression in the vaccinated cells. Moreover, codons may be selected which are most efficiently transcribed in the vaccinated cell. Examples of polyadenylation signals useful for practicing this aspect of the present invention (especially for a genetic vaccine intended for use in humans), include, but are not limited to SV40 polyadenylation signals and LTR polyadenylation signals.

In addition to the regulatory elements required for DNA expression, other elements may also be included in the DNA molecule. Such additional elements include enhancers. The enhancer may be selected from the group including, but not limited to, the human actin enhancer, human myosin enhancer, CMV enhancer, RSV enhancer, human hemoglobin enhancer, human muscle creatine enhancer, and EBV enhancer.

Genetic constructs may comprise a mammalian origin of replication, the activity of which serves to produce multiple copies of the construct in the recipient cell and thereby, maintain the construct extrachromosomally. Plasmids pCEP4 and pREP4 (Invitrogen, San Diego, Calif.) comprise the EBV origin of replication and nuclear antigen EBNA-1 coding region and achieve high copy episomal replication in the relative absence of integration. Such plasmids may be used in accordance with the invention.

An additional element may be added which serves as a target for cell destruction if it is desirable to eliminate cells receiving the genetic construct for any reason. A herpes thymidine kinase (tk) gene in an expressible form can be included in the genetic construct. When the construct is introduced into the cell, tk will be produced. The drug gangcyclovir can subsequently be administered to the individual, administration of which causes the selective killing of any cell producing tk. Thus, a system is provided which allows for the selective destruction of vaccinated cells.

The immunogenicity of genetic vaccines may also be augmented by rendering them “self-replicating”. RNA vectors encoding an RNA replicase, a polypeptide derived from alphaviruses (such as, e.g., Sindbis virus), are significantly more immunogenic than conventional plasmids. Cells into which a construct comprising an antigen and an RNA replicase has been introduced briefly produce large amounts of antigen before undergoing apoptotic death. DNA and RNA-based vaccines and methods of use are described in detail in several publications, including Leitner et al. (1999, Vaccines 18:765-777), Nagashunmugam et al. (1997, AIDS 11:1433-1444), and Fleeton et al. (2001, J Infect Dis 183:1395-1398).

DNA and RNA vaccines may also be rendered more effective by enhancing their uptake into antigen presenting cells, which in turn leads to activation of the cellular immune response, including killer T cells.

In order to test expression, genetic constructs can be tested for expression levels in vitro using tissue culture cells of the same type as those intended to be vaccinated. For example, if the genetic vaccine is to be administered into human muscle cells, muscle cells grown in culture such as solid muscle tumor cells of rhabdomyosarcoma may be used as an in vitro model to measure expression level. One of ordinary skill in the art could readily identify a model in vitro system which may be used to measure expression levels of an encoded target protein.

In accordance with the invention, multiple inoculants can be delivered to different cells, cell types, or tissues in an individual. Such inoculants may comprise the same or different nucleic acid sequences of a pathogenic organism. This allows for the introduction of more than a single antigen target and maximizes the chances for development of immunity to the pathogen in a vaccinated subject.

According to the invention, a genetic vaccine may be introduced in vivo into cells of an individual to be immunized or ex vivo into cells of the individual which are re-implanted after incorporation of the genetic vaccine. Either route may be used to introduce genetic material into cells of an individual. Preferred routes of administration include intramuscular, intraperitoneal, intradermal, and subcutaneous injection. Alternatively, the genetic vaccine may be introduced by various means into cells isolated from an individual. Such means include, for example, transfection, electroporation, and microprojectile bombardment. These methods and other protocols for introducing nucleic acid sequences into cells are known to and routinely practiced by skilled practitioners. After the genetic construct is incorporated into the cells, they are re-implanted into the individual. Prior to re-implantation, the expression levels of a target protein encoded by the genetic vaccine may be assessed. It is contemplated that otherwise non-immunogenic cells that have genetic constructs incorporated therein can be implanted into autologous or heterologous recipients.

Genetic vaccines generally comprise about 0.1 to about 1000 micrograms of nucleic acid sequences (i.e., DNA or RNA). In particular embodiments, the vaccines contain about 1 to about 500 micrograms of nucleic acid sequences. In more particular embodiments, the vaccines contain about 25 to about 250 micrograms of nucleic acid sequences. Most particularly, the vaccines contain about 100 micrograms nucleic acid sequences.

The genetic vaccines are formulated according to the intended mode of administration. A skilled practitioner can readily formulate a genetic vaccine that comprises a genetic construct. In cases where intramuscular injection is the chosen administration mode, an isotonic formulation is usually used. Generally, additives for isotonicity can include sodium chloride, dextrose, mannitol, sorbitol and lactose. Isotonic solutions such as phosphate buffered saline are preferred. Stabilizers can include gelatin and albumin.

According to the present invention, prior to or contemporaneously with administration of a genetic construct, cells may be treated with a “cell stimulating” or “cell proliferative” agent. As used herein, the terms “cell stimulating agent” or “cell proliferative agent” are used interchangeably and refer to compounds which stimulate cell division. Such compounds facilitate DNA and RNA uptake. Contemplated cell stimulating agents include lectins, growth factors, cytokines and lymphokines such as platelet derived growth factor (PDGF), granulocyte-colony stimulating factor (G-CSF), granulocyte/macrophage-colony stimulating factor (GM-CSF), epidermal growth factor (EGF) and IL-4.

A genetic construct may be combined with collagen as an emulsion and delivered intraperitoneally. The collagen emulsion provides a means for sustained release of DNA. 50 μl to 2 ml of collagen are used. About 100 μg DNA are combined with 1 ml of collagen in a particular embodiment using this formulation.

In some embodiments of the invention, the individual is administered a series of vaccinations to produce a comprehensive immune response. According to this method, at least two and preferably four injections are given over a period of time. The period of time between injections may include from 24 hours apart to two weeks or longer between injections, preferably one week apart. Alternatively, at least two and up to four separate injections may be administered simultaneously at different parts of the body.

In some embodiments of the invention, a complete vaccination includes injection of two or more different inoculants into different tissues. For example, in a viral vaccine according to the invention, the vaccine comprises two inoculants in which each one comprises genetic material encoding a different viral protein(s). This method of vaccination allows the introduction of a spectrum of viral genes into the individual without the risk of assembling an infectious viral particle. Thus, an immune response against most or all of the immunogenic components of a virus can be invoked in the vaccinated individual. Injection of each inoculant may be performed at different sites, preferably at a distance, to ensure that different genetic constructs are not introduced into the same cell.

The present invention relates to a method of immunizing an individual against a pathogen comprising the steps of administering to cells of the individual a DNA molecule that comprises a nucleic acid sequence encoding a pathogen antigen operatively linked to regulatory sequences, wherein the nucleic acid sequence is capable of being expressed in the cells. The method may further comprise contacting the cells with a cell stimulating agent. In one embodiment, the individual is a human. In particular embodiments, the nucleic acid molecule is administered to cells in vivo. The nucleic acid molecule may be administered intramuscularly.

Virus-like particles. In the context of the present application, virus-like particles (VLPs) are membrane-surrounded structures comprising at least one viral surface protein embedded within the membrane of the host cell in which the VLPs are produced. VLPs do not contain intact viral nucleic acid and are, therefore, non-infectious. Exemplary VLPs of the invention include virus-like particles of any pathogenic virus of interest. Preferably, there is sufficient viral surface protein on the surface of the VLP so that when a VLP preparation is formulated into an immunogenic composition and administered to an animal or human, an immune response (cell-mediated and/or humoral) is elicited. The viral surface protein may be a full length polypeptide, or a truncate, variant, modified polypeptide thereof. Such polypeptides should retain at least one surface antigenic determinant against which an immune response may be generated, preferably a protective immune response.

Methods for the production of VLPs carrying immunogenic viral surface proteins (or truncates, variants, or modified polypeptides thereof) of pathogenic viruses are known in the art. Viruses whose cell surface protein coding sequences can be adapted for expression in baculovirus expression systems, preferably Autographa californica nuclear polyhedrosis virus (AcNPV) expression vectors in which the viral protein(s) is expressed under the regulatory control of a late or late/very late hybrid promoter, include any pathogenic virus capable of infecting a subject of the invention.

Modified Virus

According to the present invention, any clinical isolate of at least one strain of a particular virus may be used as starting material from which to derive modified viruses, and nucleic and amino acid sequences.

According to methods for replicating modified viruses of the present invention, suitable mammalian host cells can be used, including Vero cells or other mammalian cells suitably excluding adventitious agents, preferably of a suitable passage number that can be certified according to the WHO requirements for vaccine production (Mizrahi, ed., Viral Vaccines, Wiley-Liss, New York (1990), pp. 39-60). Non-limiting examples of cell lines that are suitable for methods, viruses and compositions used in the present invention, include, but are not limited to, mammalian fibroblast or cultured epithelial cells as continuous cell lines. Further non-limiting examples include Vero (e.g., Vero E6), MDBK, BK-21 and CV-1 cells, readily available from commercial sources (e.g., ATCC, Rockville, Md.). Vero cells of passage number less than 191 are preferred, or any range or value therein.

Continuous cell lines (CCLs), derived from primary diploid cells, may be used for replicating a virus according to the present invention. CCLs possess advantages over primary diploid cells, such as suitability for large-scale cultivation; high sensitivity to different viral variants; unrestricted and stable growth; and low cost (relative to primary diploid cell cultures). Montagnon et al., Dev. Biol. Stand. 47:55 (1987); Grachev, Virol. 4:44 (1983); Smith et al., J. Clin. Microbiol. 24:265 (1986); Grachev et al., in Guidance for the Production of Vaccines and Sera, Burgamov, ed., Medicine, Moscow p. 176 (1978)).

WHO certified, or certifiable, continuous cell lines are preferred for producing virus vaccines of the present invention. The requirements for certifying such cell lines include characterization with respect to at least one of genealogy, growth characteristics, immunological markers, virus susceptibility, tumorigenicity and storage conditions, as well as by testing in animals, eggs, and cell culture. Such characterization is used to confirm that the CCLs are free from detectable adventitious agents. In some countries, karyology may also be required. In addition, tumorigenicity is preferably tested in cells that are at the same passage level as those used for vaccine production. The replicated virus is preferably purified by a process proven to give consistent results, before being inactivated or attenuated for vaccine production (see, e.g., World Health Organization TRS No. 673 (1982)).

In general, a complete characterization of the continuous cell line to be used is established, so that appropriate tests for purity of the final product can be included. Data of utility for the characterization of a continuous cell line for use in the present invention include (a) information on its origin, derivation, and passage history; (b) information on its growth and morphological characteristics; (c) results of tests of adventitious agents; (d) distinguishing features, such as biochemical, immunological, and cytogenetic patterns which allow the cells to be definitively recognized among other cell lines; and (e) results of tumorigenicity tests. Preferably, the passage level, or population doubling, of the cell line used is as low as possible.

It is preferred that the replicated virus produced in continuous cell lines is highly purified prior to vaccine formulation. Purification procedures generally result in the extensive removal of cellular DNA, other cellular components, and adventitious agents. Procedures that extensively degrade or denature DNA can also be used. See, e.g., Mizrahi, ed., Viral Vaccines, Wiley-Liss, New York pp. 39-67 (1990).

Identifying Modified Virus of Utility in Vaccine Compositions

The screening of modified viruses for use in vaccine production, can be performed using any known and/or suitable assay, as is known in the art. Such assays (alone or in any combination) that are suitable for screening include, but are not limited to, viral replication, quantitative and/or qualitative measurement of inactivation (e.g., by antisera), transcription, replication, translation, virion incorporation, virulence, viral yield, and/or morphogenesis, using such methods as reverse genetics, reassortment, complementation, and/or infection. For example, virus replication assays can be used to screen for attenuation or inactivation of the virus.

A resulting modified virus may be concentrated and/or purified (e.g., by centrifugation or column chromatography) and then inactivated or attenuated using known method steps.

Vaccine Formulations Using Modified Virus

The invention encompasses vaccine formulations comprising wildtype or modified nucleic acid sequences encoding a viral polypeptide or a truncate or antigenic epitope thereof; expression vectors comprising such nucleic acid sequences; the amino acid sequences of a wildtype or modified polypeptide or a truncate or antigenic epitope thereof; VLPs; and modified viruses. The invention encompasses the use of at least one of the above in vaccine formulations to confer protection against a virus infection.

Vaccine formulations of the invention may be administered in combination to confer protection against viral infection. Such combination therapy may employ simultaneous or temporally divergent administration protocols.

Combination therapy also encompasses the administration of a vaccine formulation of the invention with nucleic acid sequences encoding proteins with immunopotentiating activities, expression vectors comprising such nucleic acid sequences, or an immunopotentiating polypeptide or a functional fragment thereof. Examples of immunopotentiating proteins include, but are not limited to, cytokines, interferon type 1, gamma interferon, colony stimulating factors, and interleukin-1, -2, 4, -5, -6, -12.

The invention encompasses vaccine formulations to be administered to humans and animals. In particular, the invention encompasses vaccine formulations to be administered to humans, primates, horses, cows, sheep, pigs, goats, dogs, cats, avian species, fish, and rodents and other animals.

Vaccines

Recombinant vaccines provide an additional means for immunizing against pathogens. There are two basic types of recombinant vaccines: one is a pathogen in which specific genes are deleted in order to render the resulting agent non-virulent. Essentially, this type of recombinant vaccine is attenuated by design and generally requires the administration of an active, non-virulent infective agent which, upon establishing itself in a host, produces or causes to be produced antigens used to elicit the immune response. The second type of recombinant vaccine employs non-virulent vectors which carry genetic material encoding target antigens. This type of recombinant vaccine similarly requires the administration of an active infective non-virulent agent which, upon establishing itself in a host, produces or causes to be produced, the antigen used to elicit the immune response. Such vaccines essentially employ non-virulent agents to produce pathogen antigens that can then serve as targets for an anti-pathogenic immune response. For example, the development of vaccinia as an expression system for vaccination has theoretically simplified the safety and development of infectious vaccination strategies with broader T-cell immune responses.

In addition to vaccinia viral vectors, a number of other non-virulent live viral vectors have been used successfully as vehicles for vaccination. See, for example, Hansson et al. Biotechnol. Appl. Biochem. 32:95-107 (2000). Such live viral vectors include, but are not limited to: adenovirus, adenovirus associated virus (AAV), paramyxovirus, and poxvirus.

Recombinant vaccines require the introduction of an active infective agent which, in some cases, may lead to undesirable effects. Furthermore, in cases where the recombinant vaccine is the result of deletion of genes essential for virulence, such functionally isolatable genes must exist and be identified. In vaccines in which pathogen genes are inserted into non-virulent vectors, many problems exist which are related to the immune response elicited against the vector antigens. Such reactions can negatively impact the immune response elicited against the target antigen. A recombinant vaccine may, for example, introduce a number of vector antigens against which the immune system may respond. Moreover, a vector can only be used once per individual since, after the first exposure, the individual will develop immunity to the vector. These problems are both present, for example, in recombinant vaccines that employ adenoviral vectors. Moreover, once vaccinated with an adenoviral vector, the vaccine cannot be effectively vaccinated using the adenoviral vector again. Such potentially adverse aspects associated with recombinant vectors are well known in the art and would be taken into consideration when designing a recombinant viral vector of the present invention.

Active Immunization

Either a live recombinant viral vaccine or an inactivated recombinant viral vaccine can be formulated. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification. The extent of viral modification required to ensure its safety for use as a live vaccine depends on the pathogenicity of the virus for the animal species intended as the subject for immunization.

In this regard, genetically engineered or modified viruses and/or vectors may be used effectively to screen for and identify target sequences for mutation, whose presence in mutated form would confer attenuation characteristics to a particular strain. The introduction of appropriate mutations (e.g., deletions) into templates used for transfection may provide the novel viruses with attenuation characteristics. For example, specific missense mutations which are associated with temperature sensitivity or cold adaptation can be incorporated into deletion mutations. These mutations should be more stable than the point mutations associated with cold or temperature sensitive mutants and reversion frequencies should be extremely low.

Alternatively, modified viruses with “suicide” characteristics may be constructed. Such viruses are capable of only one or a few rounds of replication within the host. When used as a vaccine, such an impaired recombinant virus undergoes a limited number of replication cycle(s), sufficient to produce enough viral particles to induce a productive immune response, but incapable of causing disease in the human host. Recombinant viruses lacking one or more of the essential viral genes or possessing mutated viral genes, for example, could be identified that are unable to undergo successive rounds of replication. Defective viruses can be produced in cell lines which permanently express the defective gene(s). Viruses lacking an essential gene(s) will be replicated in these cell lines but when administered to the human host will not be able to complete a round of replication. Such preparations may, during this abortive cycle, transcribe and translate a sufficient number of genes to induce an immune response. Alternatively, larger quantities of the strains could be administered, so that these preparations serve as inactivated (killed) virus vaccines. For inactivated vaccines, it is preferred that a modified gene product be expressed as a viral component, so that the gene product is associated with the virion. The advantage of such preparations is that they contain native proteins and do not undergo inactivation by treatment with formalin or other agents generally used in the manufacture of killed virus vaccines. Alternatively, mutated virus made from cDNA may be highly attenuated so that it replicates for only a few rounds.

Inactivated Vaccines. In another embodiment of the invention, inactivated vaccine formulations may be prepared using conventional techniques to “kill” the modified viruses. Inactivated vaccines are “dead” in the sense that their infectivity has been destroyed. Ideally, the infectivity of the virus is destroyed without affecting its immunogenicity. In such vaccines, the pathogen is either killed or otherwise inactivated using means such as, for example, heat or chemicals. The administration of killed or inactivated pathogen into an individual presents the pathogen to the individual's immune system in a noninfective form and the individual can thereby mount an immune response against it. Killed or inactivated pathogen vaccines provide protection by directly generating T-helper and humoral immune responses against the pathogenic immunogens. Because the pathogen is killed or otherwise inactivated, there is little threat of infection.

In order to prepare inactivated vaccines, a modified virus may be grown in cell culture or in the allantois of the chick embryo, purified by zonal ultracentrifugation, inactivated by formaldehyde or beta-propiolactone, and pooled. The resulting vaccine is usually inoculated intramuscularly. Inactivated vaccine types that can be used in the invention can include whole-virus (WV) vaccine or subvirion (SV) virus vaccine. The WV vaccine contains intact, inactivated virus, while the SV vaccine contains purified virus disrupted with detergents that solubilize the lipid-containing viral envelope, followed by chemical inactivation of residual virus.

It has been observed, however, that absent an active infection, a complete immune response may not be elicited. Killed and inactivated vaccines, for example, do not reproduce or otherwise undergo an infective cycle, and thus, do not generally elicit the CIL arm of the cellular immune response. Absent the CIL arm, the immune response elicited by a killed or inactivated vaccine may be insufficient to adequately protect an individual. Additionally, killed and inactivated vaccines are sometimes altered by the means used to render them inactivated. These changes can sometimes affect the immunogenicity of the antigens. Such considerations are known to skilled practitioners and would, accordingly, be considered with respect to aspects of the present invention.

Inactivated viruses may be formulated with a suitable adjuvant in order to enhance the immunological response. Such adjuvants may include but are not limited to mineral gels, e.g., aluminum hydroxide; surface active substances such as lysolecithin, pluronic polyols, polyanions; peptides; oil emulsions; and potentially useful human adjuvants such as BCG and Corynebacterium parvum.

Live Attenuated Virus Vaccines. Another method for vaccinating against pathogens is to provide an attenuated vaccine. Attenuated vaccines are essentially live vaccines which exhibit a reduced infectivity. Attenuated vaccines are often produced by passaging several generations of the pathogen through a permissive host until the progeny agents are no longer virulent. By using an attenuated vaccine, an agent that displays limited infectivity may be employed to elicit an immune response against the pathogen. By maintaining a certain level of infectivity, the attenuated vaccine produces a low level infection and elicits a stronger immune response than killed or inactivated vaccines. For example, live attenuated vaccines, such as the poliovirus and smallpox vaccines, stimulate protective T-helper, T-cytotoxic, and humoral immunities during their nonpathogenic infection of the host.

Live, attenuated virus may be used to advantage in vaccines for preventing or treating virus infection, according to known method steps. Attenuation may be achieved in a single step by transfer of attenuating genes from an attenuated donor virus to a replicated isolate or reasserted virus according to known methods (see, e.g., Murphy, Infect. Dis. Clin. Pract. 2:174-181 (1993)).

Attenuated viruses generated by reverse genetics approaches, for example, may be used in the vaccine and pharmaceutical formulations described herein. Reverse genetics techniques can also be used to engineer additional mutations to other viral genes important for vaccine production, for example, the epitopes of useful vaccine strain variants can be engineered into the attenuated virus. Alternatively, epitopes that alter the cellular tropism of the virus in vivo can be engineered into an attenuated virus of the invention.

Other attenuating mutations can be introduced into particular virus genes by site-directed mutagenesis to rescue infectious viruses bearing these mutant genes. Attenuating mutations can be introduced into non-coding regions of the genome, as well as into coding regions. Thus, new donor viruses can also be generated bearing attenuating mutations introduced by site-directed mutagenesis, and such new donor viruses can be used in the production of live attenuated reassortants.

It is preferred that such attenuated viruses maintain the genes from the replicated virus that encode antigenic determinants substantially similar to those of the original clinical isolates. This is because the purpose of the attenuated vaccine is to provide substantially the same antigenicity as the original clinical isolate of the virus, while at the same time lacking infectivity to the degree that the vaccine causes minimal chance of inducing a serious pathogenic condition in the vaccinated mammal.

As described hereinabove, attenuated vaccines often make very effective vaccines because they are capable of a limited, non-virulent infection and result in immune responses involving a humoral response and both arms of the cellular immune response. There are, however, several problems associated with attenuated vaccines. Firstly, it is difficult to test attenuated vaccines to determine when they are no longer pathogenic. The risk of the vaccine being virulent is often too great to properly test for effective attenuation. Secondly, attenuated vaccines carry the risk of reverting into a virulent form of the pathogen. Thus, there is a risk of infecting individuals with a virulent form of the pathogen when using an attenuated vaccine. A skilled practitioner would be aware of these issues and incorporate precautions to address these issues into the design of an attenuated viral vaccine.

The replicated virus can thus be attenuated or inactivated, formulated and administered, according to known methods, as a vaccine to induce an immune response in a mammal. Methods are well known in the art for determining whether such attenuated or inactivated vaccines have maintained similar antigenicity to that of the clinical isolate or variant strain derived therefrom. Such known methods include the use of antisera or antibodies to eliminate viruses expressing antigenic determinants of the donor virus; chemical selection (e.g, amantadine or rimantidine); surface protein activity and/or inhibition; and DNA screening (such as probe hybridization or PCR) to confirm that donor genes encoding the antigenic determinants are not present in the attenuated viruses. See, e.g., Kilbourne, Bull. M2 World Health Org. 41:643-645 (1969); Aymard-Henry et al., Bull. World Health Org. 481:199-202 (1973); Mahy et al., J. Biol. Stand. 5:237-247 (1977); Barrett et al., Virology: A Practical Approach, Oxford IRL Press, Oxford, pp. 119-150 (1985); Robertson et al., Biologicals 20:213-220 (1992).

Pharmaceutical Compositions

Many methods may be used to introduce the vaccine formulations described above, these include but are not limited to oral, topical, intradermal, intramuscular, intraperitoneal, intravenous, subcutaneous, intra-pulmonary, rectal, vaginal and intranasal routes. All such routes are suitable for administration of these compositions, and may be selected depending on the patient and condition treated if there is a condition present, and similar factors by an attending physician. It may be preferable to introduce a vaccine formulation via the natural route of infection of the pathogen for which the vaccine is designed. According to the desired route for administration, the compositions of the invention are prepared in the form of, for example, liquids, powders, aerosols, tablets, capsules, enteric coated tablets or capsules, or suppositories.

In general, selection of the appropriate dosage for the priming compositions of the present invention will be based upon the physical condition of the mammal, most especially including the general health and weight of the immunized mammal. Such selection and upward or downward adjustment of the effective dose is within the skill of the art.

Pharmaceutical compositions of the present invention, suitable for inoculation or for parenteral or oral administration, comprise attenuated or inactivated forms of mammalian viruses, for example, optionally further comprising sterile aqueous or non-aqueous solutions, suspensions, and emulsions. The composition can further comprise auxiliary agents or excipients, as known in the art. See, e.g, Berkow et al., eds., The Merck Manual, 15th edition, Merck and Co., Rahway, N.J. (1987); Goodman et al., eds., Goodman and Gilman's The Pharmacological Basis of Therapeutics, 8th edition, Pergamon Press, Inc., Elmsford, N.Y. (1990); Avery's Drug Treatment: Principles and Practice of Clinical Pharmacology and Therapeutics, 3rd edition, ADIS Press, LTD., Williams and Wilkins, Baltimore, Md. (1987); Osol, A., ed., Remington's Pharmaceutical Sciences, Mack Publishing Co, Easton, Pa. pp. 1324-1341 (1980); Katzung, ed. Basic and Clinical Pharmacology, Fifth Edition, Appleton and Lange, Norwalk, Conn. (1992), which references and references cited therein, are entirely incorporated herein by reference as they show the state of the art.

A virus vaccine composition of the present invention can comprise from about 10²-10⁹ plaque forming units (PFU)/ml, or any range or value therein, where the virus is attenuated. A vaccine composition comprising an inactivated virus can comprise an amount of virus corresponding to about 0.1 to 200 micrograms of an antigenic protein/ml or combinations thereof, or any range or value therein.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow, infra, Goodman, infra, Avery's, infra, Osol, infra and Katzung, infra, which are incorporated in their entirety herein by reference.

A vaccine composition of the present invention, used for administration to an individual, may further comprise salts, preservatives, chemical stabilizers, buffers, adjuvants, or other substances which are desirable for improving the efficacy of the composition. Typically, stabilizers, adjuvants, and preservatives are optimized to determine the best formulation for efficacy in the target human or animal. Suitable exemplary preservatives include chlorobutanol potassium sorbate, sorbic acid, sulfur dioxide, propyl gallate, the parabens, ethyl vanillin, glycerin, phenol, and parachlorophenol. Suitable stabilizing ingredients which may be used include, for example, casamino acids, sucrose, gelatin, phenol red, N-Z amine, monopotassium diphosphate, lactose, lactalbumin hydrolysate, and dried milk. Adjuvants are substances that can be used to attract leukocytes or augment a specific immune response. Normally, the adjuvant and the composition are mixed prior to presentation to the immune system, or presented separately, but into the same site of the mammal being immunized. Such adjuvants include, among others, MPL™ (3-O-deacylated monophosphoryl lipid A; RIBI ImmunoChem Research, Inc., Hamilton, Mont.), mineral oil and water, aluminum hydroxide, Amphigen, Avridine, L121/squalene, D-lactide-polylactide/glycoside, pluronic plyois, muramyl dipeptide, killed Bordetella, saponins, such as Quil A or Stimulon™ QS-21 (Aquila Biopharmaceuticals, Inc., Framingham, Mass.) and cholera toxin (either in a wild-type or mutant form, e.g., wherein the glutamic acid at amino acid position 29 is replaced by another amino acid, preferably a histidine, in accordance with International Patent Application No. PCT/US99/22520, incorporated herein by reference). Additional examples of materials suitable for use in vaccine compositions are provided in Osol, A., ed., Remington's Pharmaceutical Sciences, Mack Publishing Co, Easton, Pa. (1980), pp. 1324-1341, which reference is incorporated in its entirety herein by reference.

Heterogeneity in the vaccine may be provided by mixing different modified viruses of the invention, such as 2-50 modified viruses or any range or value therein.

A pharmaceutical composition according to the present invention may further or additionally comprise at least one viral chemotherapeutic compound, including, but not limited to, gamma globulin, amantadine, ribavirin, guanidine, hydroxybenzimidazole, interferon-alpha, interferon-beta, interferon-gamma, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir (neuraminidase inhibiting drugs oseltamivir, zanamivir). See, e.g., Katzung, infra, and the references cited therein on pages 798-800 and 680-681, respectively, which references are herein entirely incorporated by reference.

The vaccine can also contain variable but small quantities of endotoxin, free formaldehyde, and preservative, which have been found safe and not contributing to the reactogenicity of the vaccines for humans.

Pharmaceutical Purposes

The administration of a vaccine composition may be for either a “prophylactic” or “therapeutic” purpose. When provided prophylactically, the compositions are provided before any symptom of viral infection becomes manifest. The prophylactic administration of the composition serves to prevent or attenuate any subsequent infection. When provided therapeutically, the attenuated or inactivated viral vaccine is provided upon the detection of a symptom of actual infection. The therapeutic administration of the compound(s) serves to attenuate any actual infection. See, e.g, Berkow, infra, Goodman, infra, Avery, infra and Katzung, infra, which are entirely incorporated herein by reference.

An attenuated or inactivated vaccine composition of the present invention may thus be provided either before the onset of infection (so as to prevent or attenuate an anticipated infection) or after the initiation of an actual infection.

A composition is said to be “pharmacologically acceptable” if its administration can be tolerated by a recipient patient. Such an agent is said to be administered in a “therapeutically effective amount” if the amount administered is physiologically significant. A vaccine or composition of the present invention is physiologically significant if its presence results in a detectable change in the physiology of a recipient patient that enhances at least one primary or secondary humoral or cellular immune response against at least one strain of an infectious virus.

The “protection” provided need not be absolute, i.e., the viral infection need not be totally prevented or eradicated, if there is a statistically significant improvement compared with a control population or set of patients. Protection may be limited to mitigating the severity or rapidity of symptom onset of viral infection.

Pharmaceutical Administration

In general, a vaccine may confer resistance to one or more viral strains by either passive immunization or active immunization. In active immunization, an inactivated or attenuated live vaccine composition is administered prophylactically, according to a method of the present invention.

In a second embodiment, the vaccine is provided to a mammalian female (at or prior to pregnancy or parturition), under conditions of time and amount sufficient to cause the production of antisera which serve to protect both the female and the fetus or newborn (via passive incorporation of the antibodies across the placenta or in the mother's milk).

The present invention thus includes methods for preventing or attenuating infection by at least one virus strain. As used herein, a vaccine is said to prevent or attenuate a disease if its administration results either in the total or partial attenuation (i.e., suppression) of a symptom or condition of the disease, or in the total or partial immunity of the individual to the disease.

At least one inactivated or attenuated virus, or composition thereof, of the present invention may be administered by any means that achieve the intended purpose, using a pharmaceutical composition as previously described.

For example, administration of such a composition may be by various parenteral routes such as subcutaneous, intravenous, intradermal, intramuscular, intraperitoneal, intranasal, oral or transdermal routes. Parenteral administration can be by bolus injection or by gradual perfusion over time. A preferred mode of using a pharmaceutical composition of the present invention is by intramuscular or subcutaneous application. See, e.g., Berkow, infra, Goodman, infra, Avery, infra and Katzung, infra, which are incorporated in their entirety herein by reference.

A typical regimen for preventing, suppressing, or treating a virus related pathology, may comprise administration of an effective amount of a vaccine composition as described herein, administered as a single treatment, or repeated as enhancing or booster dosages, over a period up to and including between one week and about 24 months, or any range or value therein.

According to the present invention, an “effective amount” of a vaccine composition is one that is sufficient to achieve a desired biological effect. It is understood that the effective dosage may be determined by a medical practitioner based on a number of variables including the age, sex, health, and weight of the recipient, kind of concurrent treatment, if any, frequency of treatment, and the nature of the desired outcome. The ranges of effective doses provided below are not intended to limit the invention, but are provided as representative preferred dose ranges. However, the most preferred dosage will be tailored to the individual subject, as is understood and determinable by one of skill in the art, without undue experimentation. See, e.g., Betkow et al., eds., The Merck Manual, 16th edition, Merck and Co., Rahway, N.J., 1992; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, Mass. (1985); and Katzung, infra, which references and references cited therein, are entirely incorporated herein by reference.

The dosage of an attenuated virus vaccine for a mammalian (e.g., human) adult can be from about 10³-10⁷ plaque forming units (PFU)/kg, or any range or value therein. The dose of inactivated vaccine can range from about 1 to 50 micrograms of an antigenic protein. However, the dosage should be a safe and effective amount as determined by conventional methods, using existing vaccines as a starting point.

The dosage of immunoreactive protein in each dose of virus or modified virus vaccine can be standardized to contain a suitable amount, e.g., 1-50 micrograms or any range or value therein, or an amount recommended by the U.S. Public Health Service (PHS). Each 0.5-ml dose of vaccine preferably contains approximately 1-50 billion virus particles, and preferably 10 billion particles.

While the disclosure herein is directed primarily to methods for immunizing humans, the methods of the present invention can be applied to veterinary medical uses too. It is within the scope of the present invention to provide methods of immunizing non-human as well as human subjects against pathogens and pathogen protein related disorders and diseases. Accordingly, the present invention relates to genetic immunization of mammals, birds and fish. The methods of the present invention are particularly useful for mammalian species including human, bovine, ovine, porcine, equine, canine and feline species.

While this disclosure generally discusses immunization in the context of prophylactic methods of protection, the term “immunizing” is meant to refer to both prophylactic and/or therapeutic methods. Thus, a method of immunizing includes both methods of protecting an individual from pathogen challenge, as well as methods for treating an individual suffering from pathogen infection. Accordingly, the present invention may be used as a vaccine for prophylactic protection and/or in a therapeutic manner; that is, as a reagent for immunotherapeutic methods and preparations.

Methods for Screening to Identify RIG-I Modulators and Agents Identified Thereby

The present inventors further demonstrate that retinoic acid inducible gene I (RIG-I), a dsRNA binding protein involved in innate immune responses, is one of the molecules that mediates TLR-independent recognition of viral components. Its identification in this pathway presents a molecular target for the manipulation of anti-viral immunity and the improvement of vaccine design and anti-viral therapy. The description of viral elements that enhance the activation of this pathway also suggests methods for improving vaccination efficiency.

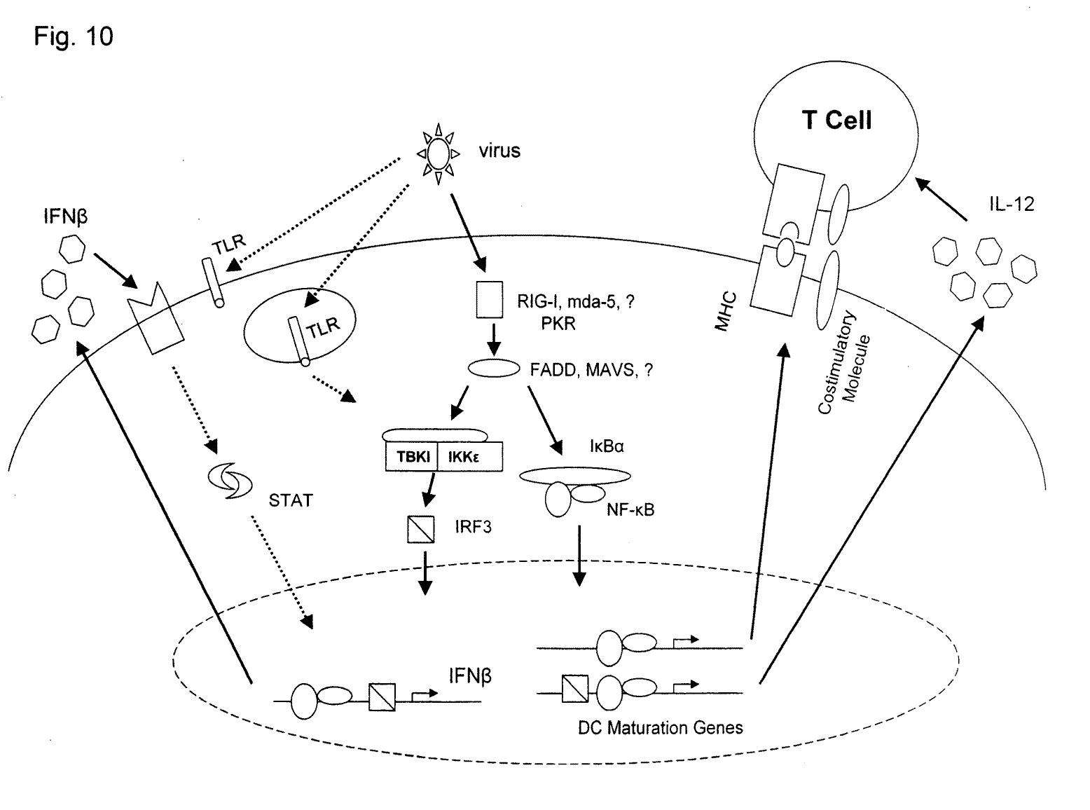

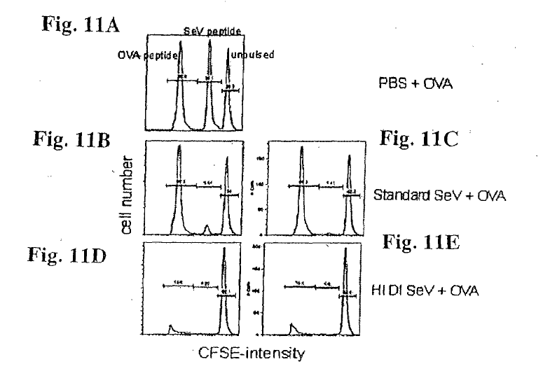

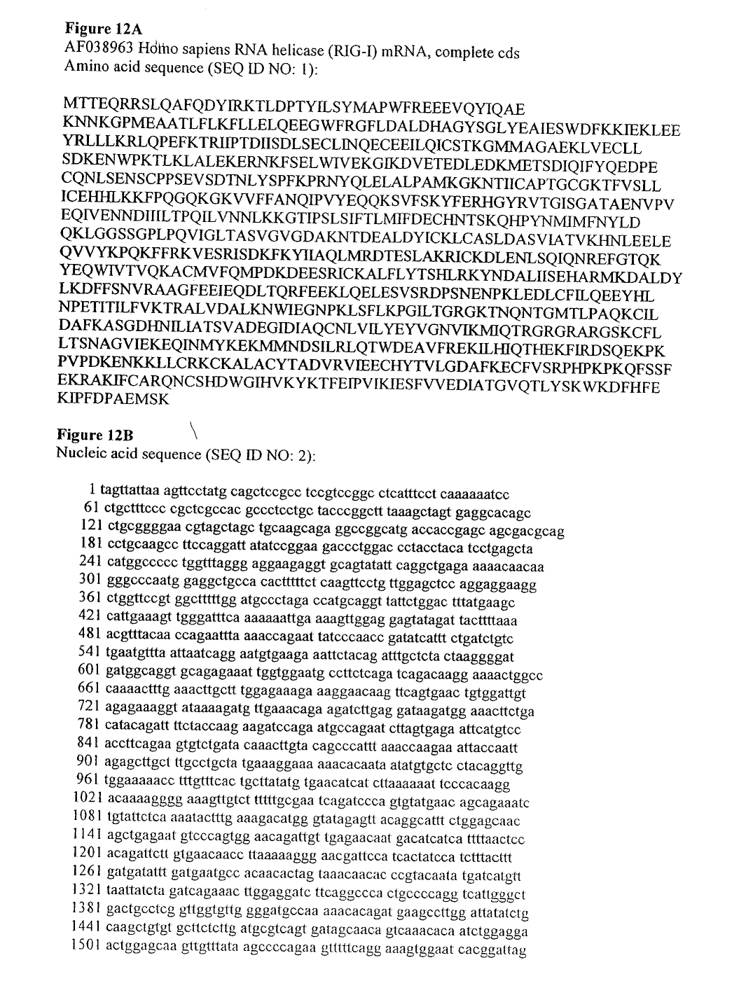

Little is presently known regarding the mechanism of RIG-I activation or molecules that act downstream of RIG-I. It is, however, known to be activated by synthetic or viral double-stranded RNA. Moreover, RIG-I induces the activation of interferon regulatory factor-3 (IRF-3), a transcription factor that participates in the induction of type I IFN. See FIG. 10 for schematic diagram of implicated signaling pathways downstream of dsRNA.

Methods for screening to identify agents capable of modulating RIG-I activity are also envisioned in the present invention. Such methods may be used to advantage to identify agents capable of either enhancing or inhibiting RIG-I activity. Identification of agents capable of enhancing RIG-I activity, which would essentially mimic TLR-independent signaling responsive to recognition of viral components, may be used to advantage to promote DC maturation. Such agents are useful in any clinical scenario in which promotion of a robust immune response is desirable. Clinical scenarios for which such agents may be used to advantage include, but are not limited to enhancement of immune response to vaccination, infectious agents, and aberrant host cells (e.g., transformed host cells).

Modulators of retinoic acid inducible gene I (RIG-I) can be identified by testing a panel of chemical compounds in a cell based system utilizing overexpression of RIG-I. Overexpression of RIG-I in cells either stably or transiently does not result in enhanced type I IFN induction until further stimuli (dsRNA/virus) is added. Adding a chemical compound capable of activating RIG-I would result in increased type I IFN induction. Thus, this assay would involve adding chemical compounds to cells overexpressing RIG-I and using type I IFN as a readout for the activation of RIG-I. Control experiments for the specificity of activation of RIG-I would involve co-expressing a RIG-I dominant negative protein to suppress the type I IFN induction effect of the chemical compound. Further confirmation of specificity can be achieved using siRNA to knock down the levels of RIG-I. Ultimately chemical modulators of RIG-I will be tested in dendritic cells expressing natural levels of RIG-I and the levels of DC maturation and type I IFN will be measured. The above protocol can be used to advantage to identify RIG-I modulators.

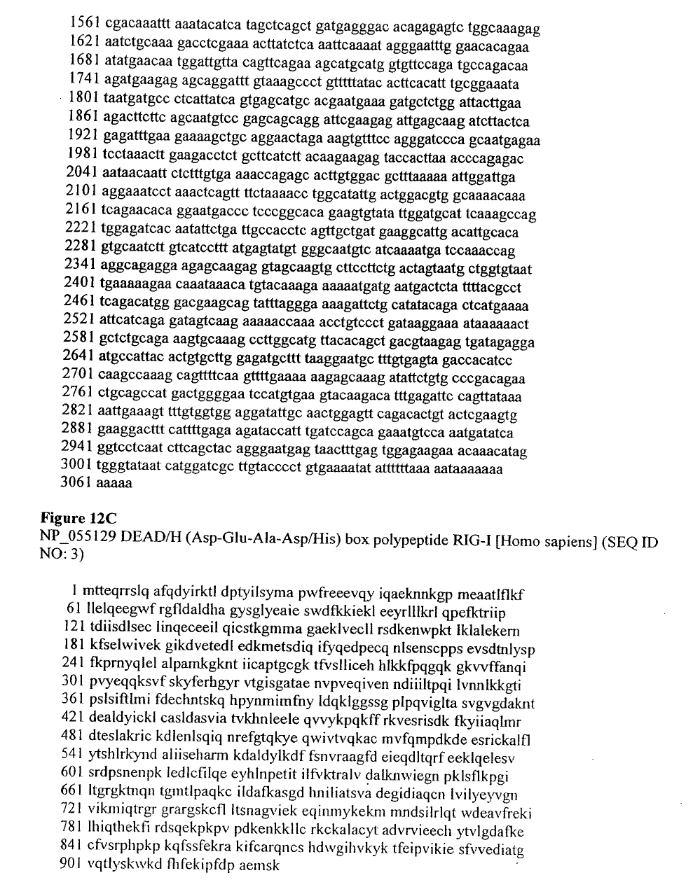

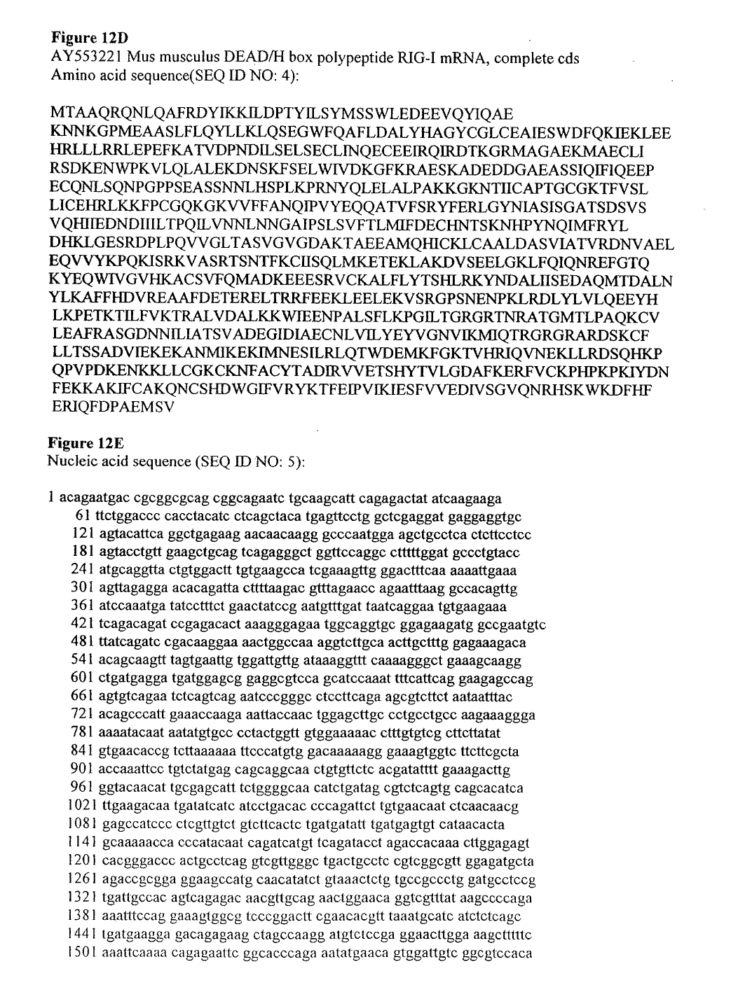

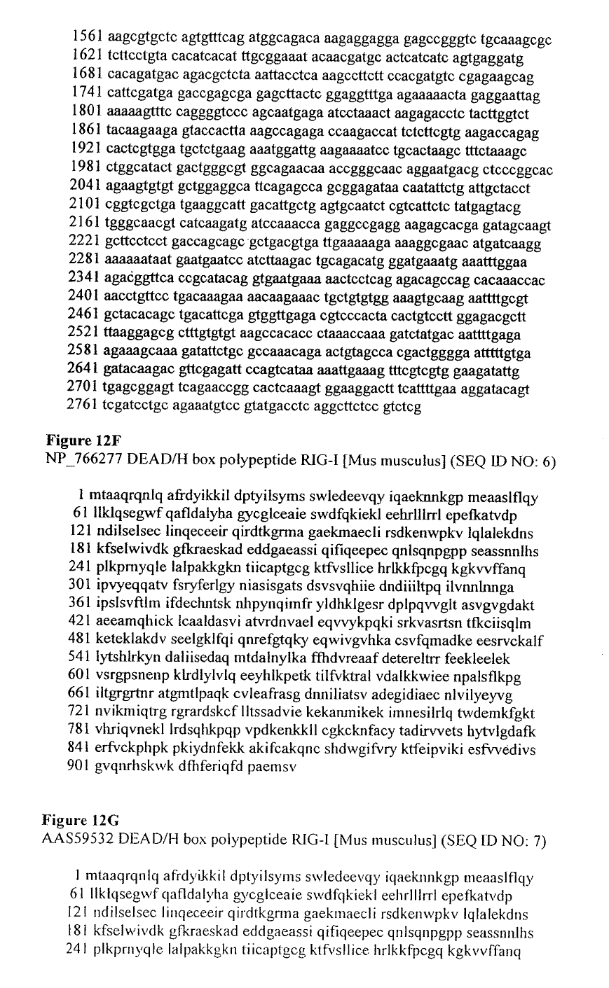

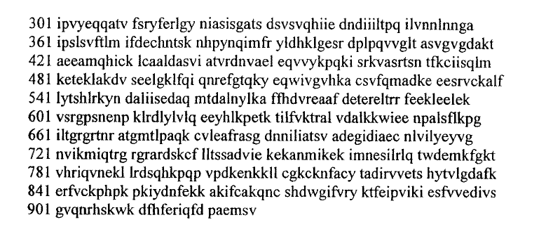

GenBank Accession numbers for full length human and mouse RIG-I sequences are as follows: full length human Rig-I cds: AF038963 (SEQ ID NOs: 1 and 2), nucleotides: NP_{—}055129 (SEQ ID NO: 3); full length mouse Rig-I cds: AY553221 (SEQ ID NOs: 4 and 5), nucleotides: Np_{—}766277 (SEQ ID NO: 6), AAS59532 (SEQ ID NO: 7). See FIG. 12 for sequences.

The invention further provides pharmaceutical compositions comprising agents capable of modulating RIG-I activity and therapeutic methods of use thereof.

### Example I

Live virus immunization provides the most complete protection against future infections and new vaccines are constantly being sought. The present inventors demonstrate herein that defective interfering (DI) particles provide enhanced immunogenic potential to standard virus preparations. Viruses with low DI particle content mature DCs poorly while DI particle amplification converts a weakly activating virus into a potent DC activator. Enhanced type I interferon induction by virus preparations high in DI particle content correlates with increased dsRNA production and requires signaling through retinoic acid inducible gene I (RIG I). Significantly, T-cell responses are considerably increased against a virus with high DI particle content as compared to its standard virus counterpart. These results demonstrate the value of DI particles in live virus vaccinations.

Materials and Methods

Viruses, Cell Lines and Mice: Influenza PR8 virus and standard SeV stocks were grown from a 1:10³ dilution in 10 day-old chicken embryonated eggs at 37° C. for 40 h. Subsequent SeV-C and SeV-52 passages at dilutions of 0, 1:10⁷, or 1:10⁶ were grown similarly for 40 h. The influenza ANSI strain was grown in 6 day-old embryonated eggs for 40 h. Allantoic fluid containing virus was harvested, snap frozen using a dry ice/ethanol bath, and stored at −80° C. Viruses were tested for bacterial contamination by inoculation on blood agar plates.

DC2.4, NIH-3T3, 293T and LLCMK2 cells were grown in tissue culture medium consisting of Dulbecco's minimum essential medium (Invitrogen, Carlsbad, Calif.), 10% fetal bovine serum (heat inactivated, endotoxin level 0.25 EU/mL; Hyclone, Logan, Utah), 1 mM sodium pyruvate, 2 mM L-glutamine (GibcoBRL, Grand Island, N.Y.), and 50 mg/nL gentamicin (Boehringer Mannheim, Indianapolis, Ind.).

C57BL/6 mice and OT-I OVA TCR transgenic mice were purchased from Taconic Farms (Germantown, N.Y.). The animals were housed in pathogen-free conditions.

Reporter Gene Assays: NIH-3T3 cells were transiently transfected with 2 ug of IFNβ promoter reporter construct [Poole et al. Virology 303, 3346 (2002)] kindly provided by Dr. D. Thanos (Columbia University, New York, N.Y.) driving firefly luciferase production together with 0.2 μg pRL-TK (Promega, Madison, Wis.) constitutively expressing renilla luciferase for normalization. To examine the role of retinoic acid inducible gene I (RIG-I), 250 ng of empty pCAGGS or pCAGGS expressing either RIG-I or the dominant negative RIG-IC [Yoneyama et al. Nat Immunol 5, 730-737 (2004)], (obtained from Drs. Washington B. Cardenas and Christopher F. Basler, Mount Sinai School of Medicine, NY) were added to the transfection mixture. Transfection was performed using Lipofectamine/Lipofectamine Plus (Invitrogen Life Technologies) according to manufacturer's instructions. Twenty four hours later, cells were infected with SeV-C or SeV-C low DI or were mock infected. Cell extracts were obtained 24 h post-infection and examined for expression of firefly and renilla luciferase using a dual luciferase assay (Promega).

To test the ability of the influenza NS1 protein to block SeV activation of the IFNβ promoter, 293T cells were transiently transfected with 1.0 μg of IFNβ promoter reporter construct, 0.1 μg pRL-TK, and 0.25 or 0.5 μg of pCAGGS empty plasmid or pCAGGS expressing the NS11-73 RNA binding domain, kindly provided by Dr. Adolfo Garcfa-Sastre (Mount Sinai School of Medicine, New York, N.Y.). Transfections were performed using TransIT-TKO transfection reagent according to manufacturer's instructions for DNA plasmids (Mirus Bio Corp, Madison, Wis.). Total DNA transfected was equalized using empty pCAGGS vector. Dual luciferase assay was performed.

Quantitative RT-PCR: RNA was extracted from DC2.4 or bone marrow derived (BM)-DCs at various time points after virus infection following manufacturer's instructions for the High Pure RNA Isolation Kit (Roche, Indianapolis, Ind.). Quantitative (q) RT-PCR was performed similarly to a previously published protocol [Yuen et al. Nucleic Acids Res 30, e48 (2002)]. In short, each sample was assayed in triplicate and the fold change values and control changes for each gene were calculated using the median threshold cycle. Primers for housekeeping genes used for normalization (rps11, GAPDH, tubulin, β-actin) were previously described [Wurmbach et al. J Biol Chem 276, 4719547201 (2001); Kislauskis et al. J Cell Biol 136, 1263-1270 (1997)]. Copy number was determined using 2500 as an empirical estimate of the number of β-actin mRNA molecules/cell [Kislauskis et al., supra]. Primer sequences used for murine IEFN and IL-12p35 genes are: IFNα5′-TCCTGAGCCAAAGTGTAGAG-3′ (SEQ ID NO: 8) and 5′-GAGAACAAGTGCCAATTACAG-3′ (SEQ ID NO: 9), IFNβ5′-AGATGTCCTCAACTGCTCTC-3′ (SEQ ID NO: 10) and 5′-AGATTCACTACCAGTCCCAG-3′ (SEQ ID NO: 11), IL-12p35 5′-ACAGCTACCTCAGCATGGTC-3 (SEQ ID NO: 12) and 5′-GACGTCTCCGCCCCTTAACA-3′ (SEQ ID NO: 13). Primer sequences for the SeV NP gene are: 5′-TGCCCTGGAAGATGAGTTAG-3′ (SEQ ID NO: 14) and 5′-GCCTGTTGGTTTGTGGTAAG-3′ (SEQ ID NO: 15).

Western Blots: Antibodies against STAT1 (E-23) and phosphorylated-STAT1 (A-2) were obtained from Santa Cruz Biotechnology (Santa Cruz, Calif.) and used according to the manufacturer's instructions. Total protein extracts were prepared in whole cell extract buffer (50 mM Tris, pH 8.0, 280 mM NaCl, 0.5% IGEPAL, 0.2 mM EDTA, 2 mM EGTA, 10% glycerol, 1 μM dithiothreitol) containing the Complete protease inhibitor cocktail (Roche). Protein content was quantified using a standard Bradford Assay. Proteins were separated by SDS-PAGE and transferred to nitrocellulose filters, immunoblotted by standard procedures, and prepared for chemiluminescent detection using Renaissance reagents according to the manufacturer's protocol (PerkinElmer Life Sciences, Boston, Mass.).

Hemagglutination and Infectivity Assays: Viruses diluted 1:2 in 0.5% chicken RBCs were incubated for 30 min at 4° C. Hemagglutination (HA) of RBCs indicates the presence of virus particles. The presence of infectious particles was evaluated by infecting LLCMK2 cells with 1:10 dilutions of the virus at 37° C. After 1 h of infection, 175 μL of media containing 2 μg/mL trypsin was added and the cells were further incubated for 72 h at 37° C. 50 μL of media was then removed from the plate and tested by HA for the presence of virus particles.

Cytokine Detection: Supernatants from infected DCs were collected 24 h after infection. IFNα secretion was measured by capture ELISA (PBL Biomedical Laboratories, Piscataway, N.J.). IL-6 and TNFα detection kits (DuoSet ELISA Development Systems) were purchased from R&D Systems (Minneapolis, Minn.). Assays were performed according to the manufacturer's protocol. The IL-12p40 ELISA was performed using capture and secondary antibodies (C15.6 and C17.8; PharMingen, San Diego, Calif.) according to the manufacturer's protocol. In some experiments supernatants from cell cultures were collected and analyzed using a multiplex bead assay for cytokines (Upstate Biotechnology, UK).

Generation of BM-DCs aid Flow Cytometry: DCs were prepared according to a standard protocol ensuring the production of immature DCs [Lopez et al. J Infect Dis 187, 1126-1136 (2003)]. Briefly, bone marrow precursors were depleted of cells expressing CD4, CD8, B220, and MHC II by magnetic bead separation and cultured in 25 units/mL GMCSF at a density of 7×10⁵ cells per well in a 24-well plate. After 4 days of incubation, all the cells in the culture expressed the CD11b marker and approximately 30% corresponded to CD11c⁺DEC205+GR1⁻ immature DCs (these cells expressed undetectable to low levels of MHC II and costimulatory molecules). The remaining cells were DC precursors (CD11b⁺ Cd11c⁻GR1⁺). Immature DCs were infected in their original wells in order to minimize maturation due to manipulation of the cells. Infection with different virus stocks was equalized using 200 HA units or MOIs of 0.5, 2 or 10. Cells were collected 24 hours post-infection and stained with FITC conjugated antibodies to CD80, CD86, or MHC II (PharMingen). Flow cytometry was performed on a Cytomics FC 500 machine (Beckman Coulter, Miami, Fla.) and analyzed using Flowjo software.

Viral RNA Isolation and DI Particle Detection by PCR: RNA was directly extracted from virus in allantoic fluid using Trizol reagent according to the manufacturer's instructions (Invitrogen Life Technologies). For detection of DI particle genomes, viral RNA was reverse transcribed using a primer specific for the SeV antigenomic promoter with an added SapI restriction site (5′-CCGGGCTCTTCGGCCACCAGACAAGAGTTTAAGAGATATTTATTC-3′; SEQ ID NO: 16). PCR was performed as described above using this single primer which can amplify in forward and reverse directions on a copy-back DI particle. As a control for the presence of standard virus genomes, a PCR was performed using the primer designed for the antigenomic promoter as well as a second primer with an added NheI restriction site (5′-GCGCGCTAGCTGTCGGTCTAAGGCAGAAAATGTGG-3′; SEQ ID NO: 17) expected to amplify 3400 bp of the L gene.

DI Particle Purification: DI particles were purified as described previously [Johnston. J Gen Virol 56, 175-184 (1981)]. In short, SeV-C was grown at the standard 1:10³ dilution in 100 eggs for 40 h. Allantoic fluid was pooled and concentrated by high speed centrifugation. Pellets were resuspended and incubated overnight at 4° C. in 0.5 mL PBS/2 mM EDTA. The virus suspension was then added to a column containing a 5-45% sucrose gradient and centrifuged at 4° C. for 1.5 h at 28,000 rpm. A pellet described to contain viral aggregates was visible as were bands representing high and low density viral particles [Johnston. supra]. The fractions containing low density viral particles were collected, pelleted, and resuspended in PBS/2 mM EDTA and applied to a second 545% sucrose gradient and centrifuged at 4° C. for 1.5 h at 28,000 rpm. Fractions were collected and analyzed by HA assay and for replication ability. The fractionated DI particles were characterized as described in the results section.

Electron Microscopy: SeV-C and pDI particles were adsorbed onto formvar-coated copper grids and negatively stained with 10 g/L phosphotungstic acid at pH 7.0. Viral particles were visualized using a Hitachi H-7000 transmission electron microscope.

In Vitro Stimulation of OT-I, OVA Specific TCR Transgenic Cells: CD8⁺ T cells were purified from the spleens of OT-I mice by negative selection. Briefly, the cells were incubated with antibodies against CD4, MHC II and B220 (BD Biosciences) followed by goat anti-rat-coated magnetic beads (Polysciences Inc., Warrington, Pa.) and magnetic separation. Enriched OT-I cells were labeled with 5,6-carboxy-succinimidyl-fluorescein-ester (CFSE) for analysis of proliferation. In brief, 10⁷ cells/ml were incubated with CFSE (Molecular Probes) at 5 μM for 10 min at 37° C. Extra CFSE was neutralized with an equal volume of FCS and the cells were washed in PBS.

To establish the co-cultures, BM-DCs were infected in the presence of OVA SIINFEKL peptide (SEQ ID NO: 18; 10 μM final concentration) for 1 h and incubated in a 1:10 or 1:20 ratio with labeled OT-I cells for 3 days. T cells were stained with anti-CD25 and anti-CD69 antibodies (BD Biosiences) and their activation and proliferation was determined by flow cytometry measuring the dilution of the CFSE dye and the expression of CD25 and CD69.

Introduction

Adaptive immune responses against viruses and other pathogens are initiated by dendritic cells (DCs) upon their interaction with pathogen-associated stimuli [Reis e Sousa. Immunity 14, 495-8 (2001); Barton & Medzhitov. Curr Opin Immunol 14, 380-3 (2002)]. This encounter triggers DC maturation leading to a change in the expression pattern of molecules including the major histocompatibility complexes, costimulatory molecules, chemokines and numerous pro-inflammatory cytokines. These changes confer upon the mature DC an enhanced ability to migrate to the lymph nodes, present antigen to T cells, and initiate adaptive immunity [Mellman & Steinman. Cell 106, 255-8 (2001); Banchereau & Steinman. Nature 392, 245-52 (1998)].

Upon virus infection, type I interferon (IFN), a family of cytokines that includes IFNα and β, plays an essential role in the innate control of virus growth and spread [Colonna et al. Curr Opin Immunol 14, 373-9 (2002)]. The production and release of type I IFN in response to virus infection is induced by the binding of the cellular transcription factors ATF-2/c-jun [Du et al. Cell 74, 887-98 (1993)], IRF3 [Juang et al. Proc Natl Acad Sci USA 95, 983742 (1998); Schafer et al. J Biol Chem 273, 2714-20 (1998); Wathelet et al. Mol Cell 1, 507-18 (1998); Sato et al. FEBS Lett 425, 112-6 (1998)], and NF-κB [Lenardo et al. Cell 57, 287-94 (1989); Visvanathan & Goodbourn. Embo J 8, 1129-38 (1989)) to the IFNβ promoter. Secreted type I IFN binds to its receptor resulting in the phosphorylation of signal transducers and activators of transcription (STAT) proteins that promote the subsequent expression of numerous antiviral genes and further amplification of type I IFN synthesis (Stark et al. Annu Rev Biochem 67, 227-64 (1998)].

Viruses are capable of inducing type I IFN and DC maturation by at least two distinct mechanisms. One, independent of virus replication, involves the recognition of viral components by Toll-like receptors (TLRs) localized in the cell membrane or in endosomal compartments [Matsumoto et al. Microbiol Immunol 48, 147-54 (2004); Heil et al. Science 303, 1526-9 (2004); Hochrein et al. Proc Natl Acad Sci USA (2004); Lund et al. Proc Natl Acad Sci USA 101, 5598-603 (2004)]. Signaling through these molecules leads to the expression of type I IFN as well as the induction of genes involved in DC maturation [Mazzoni & Segal. J Leukoc Biol 75, 721-30 (2004)]. A second mechanism involving intracellular recognition of a viral component, such as dsRNA, requires viral replication, is independent of TLR signaling and does not rely on type I IFN signaling [Honda et al. Proc Natl Acad Sci U S A 100, 10872-7 (2003); Lopez et al. J Immunol 173, 6882-9 (2004); Lopez et al. J Infect Dis 187, 1126-36 (2003)]. The TLR-independent mechanism is sufficient for the efficient maturation of DCs and the subsequent initiation of immunity [Lopez et al. supra (2004)].

Sendai virus Cantell (SeV-C) is a paramyxovirus strain known to potently induce type I IFN synthesis. Though its early history is unclear, this strain has been used extensively in type I IFN research [Cantell & Valle. Ann Med Exp Biol Fenn 43, 614 (1965); Saksela et al. Prog Med Virol 30, 78-86 (1984); Basler et al. J Virol 77, 7945-56 (2003); Izaguirre et al. J Leukoc Biol 74, 1125-38 (2003)] and for the production of purified IFNα [Cantell et al. Methods Enzymol 78, 29-38 (1981)]. Research from the laboratory of the present inventors has demonstrated that SeV-C is a much stronger inducer of both DC maturation and type I IFN production than SeV-52 or influenza virus, revealing a high correlation between the ability of a virus to induce DC maturation and the strength with which it triggers the synthesis of type I IFN [Lopez et al. supra (2003)]. The potent induction of DC maturation by SeV-C does not depend on secreted type I IFN or TLR signaling but does require intracellular virus replication [Lopez et al. supra (2004); Lopez et al. supra (2003)]. These characteristics make SeV-C especially valuable for the study of the host molecules and viral elements necessary for the efficient triggering of the TLR-independent induction of DC maturation and type I IFN by viruses.

Taking advantage of the differential abilities of the SeV strains C and 52 to induce DC maturation and type I IFN production, the present inventors investigated the properties of SeV-C that confer its unique ability to stimulate DCs. As shown herein, defective interfering (DI) particles in combination with standard virus account for the potent DC activating ability of SeV-C, likely through enhancing production of replication intermediaries. Moreover, as shown herein for the first time, facilitating the DI particle production of a virus that weakly induces DC maturation significantly enhances its ability to mature DCs. These results demonstrate that DI particles act as enhancers of viral associated molecular patterns that signal through retinoic acid inducible gene I (RIG-I), a dsRNA binding protein involved in innate immune responses [Yoneyama et al. supra (2004)], suggesting that this molecule may be involved in DC maturation pathways in addition to its known role in type I IFN induction. The enhanced DC maturation provided by DI particle-rich virus preparations culminates in significantly improved antibody and T-cell responses in vivo demonstrating the value of DI particles in enhancing the immunogenicity of viral vaccines.

### Results

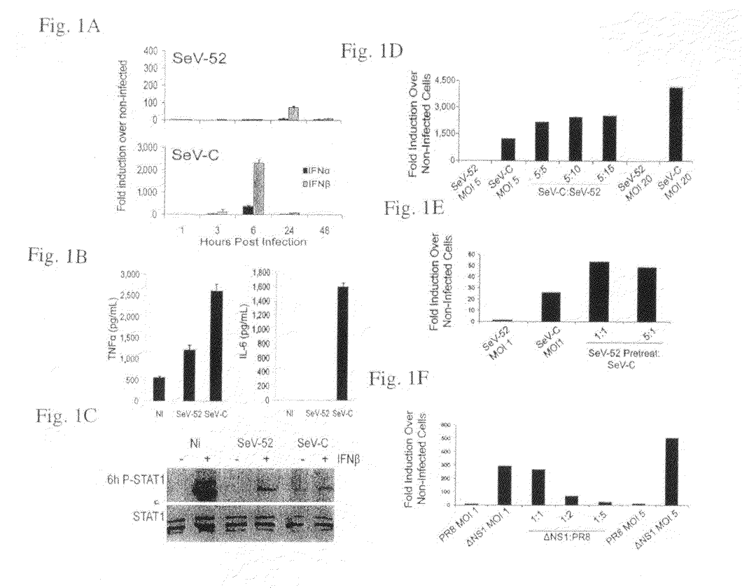

Strong DC maturation by SeV-C is not antagonized by a SeV with weak DC maturation ability: In agreement with a previous publication by the present inventors documenting the remarkable ability of SeV-C to induce IFN α secretion by BM-DCs [Lopez et al. J Infect Dis 187, 1126-1136 (2003)], SeV-C induces IFNs α and β mRNA transcription at earlier timepoints and at significantly higher levels than SeV strain 52 (FIG. 1A). Concurrently, proinflammatory cytokines indicative of DC maturation are secreted at higher concentrations by DCs infected with SeV-C than by those infected with SeV-52 (FIG. 1B and [Lopez et al. supra (2003)]). To gain insight into the mechanism by which viruses differentially trigger TLR-independent DC maturation, the present inventors focused on identifying the viral elements responsible for the different abilities of the SeV strains C and 52 to induce type I IFNs and DC maturation.

Although type I IFNs can affect various aspects of DC maturation [Gallucci et al. Nat Med 5, 1249-1255 (1999); Luft et al. J Immunol 161, 1947-1953 (1998); Santini et al. J Exp Med 191, 1777-1788 (2000)], the strong maturation induced by SeV-C occurs even in DCs lacking the type I IFN receptor [Lopez et al. supra (2003)]. In concordance with a type I IFN independent induction of DC maturation, these viruses encode four proteins, C, C′, Y1 and Y2, collectively known as the C proteins, that are able to interfere with the signaling necessary for the responsiveness to type I IFNs [Gotoh et al. Rev Med Virol 12, 337-357 (2002); Garcin et al. J Virol 77, 2321-2329 (2003)]. Accordingly, the phosphorylation of STAT1, an essential step in type I IFN signaling, is similarly inhibited by SeV-C and SeV-52 (FIG. 1C), and their C protein amino acid sequences share more than 99% identity (GenBank Accession AY909550 and AY909543). Thus, the difference between SeV-52 and SeV-C does not result from variations in the signaling of type I IFNs.

In addition to interfering with type I IFN signaling, many single-stranded RNA viruses encode antagonists able to block type I IFN synthesis. The NS1 protein of influenza virus and the V protein of SeV inhibit the induction of type I IFNs by blocking intracellular dsRNA signaling [Poole et al. Virology 303, 33-46 (2002); Garcia-Sastre et al. Virology 252, 324-330 (1998); Wang et al. J Virol 74, 11566-11573 (2000); Komatsu et al. Virology 325, 137-148 2004); Andrejeva et al. Proc Natl Acad Sci USA 101, 17264-17269 (2004). The influenza virus NS1 protein has also been shown to inhibit viral induction of DC maturation [Lopez et al. supra (2003); Fernandez-Sesma et al. J Virol In Press (2006). Thus, the present inventors hypothesized that SeV-C may encode an impaired type I IFN antagonist allowing stronger DC stimulation by viral dsRNA than SeV-52. Though divergence was found in sequences of other viral proteins, there is 100% amino acid identity between the V proteins from the 52 and C strains (GenBank Accession AY909550 and AY909543). Moreover, co-infection of the DC line DC2.4 with SeV-C and 52 did not reduce the ability of SeV-C to induce the production of IFNβ mRNA even when high MOIs of SeV-52 were used (FIG. 1D). To provide sufficient time for the synthesis of the SeV-52 type I IFN antagonist, DCs were pre-infected with SeV-52 for 18 h prior to the infection with SeV-C. SeV-52 was unable to inhibit IFNβ induction by SeV-C even under these conditions (FIG. 1E). In a similar co-infection experiment, influenza virus strain PR8, coding for the type I IFN antagonist NS1, inhibited the production of type I IFNs triggered by the antagonist deleted influenza virus ANSI (FIG. 1F). Thus, the difference between SeV-52 and SeV-C's ability to induce type I IFN and DC maturation is not explained by a mutation in the SeV-C V protein or the absence of a type I IFN antagonist similar to the influenza virus NS1.

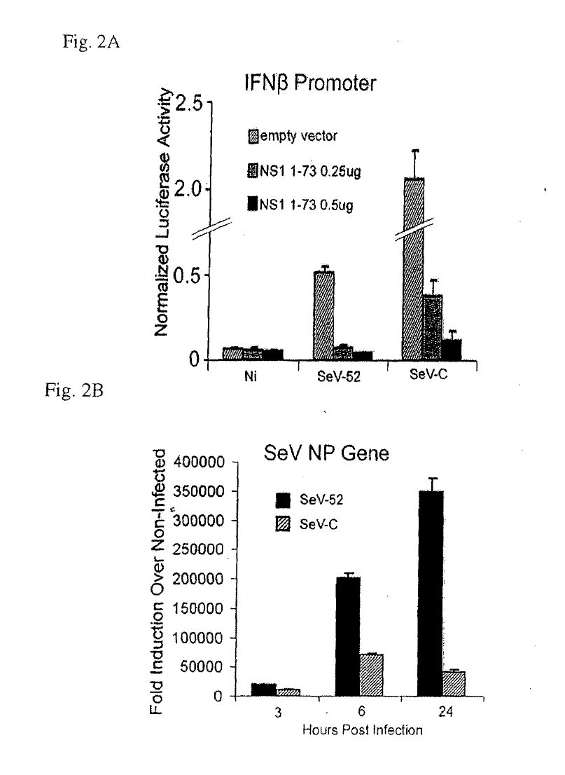

SeV-C produces more activating stimulus than SeV-52: The virus replication intermediary dsRNA has been shown to induce type I IFN synthesis through the direct or indirect activation of cellular proteins such as the dsRNA-dependent protein kinase (PKR) [Iordanov et al. Mol Cell Biol 21, 61-72 (2001], RIG-I [Yoneyama et al. supra (2004)], melanoma differentiation-associated gene 5 (mda-5) [Andrejeva et al. Proc Natl Acad Sci USA 101, 17264-17269 (2004)], the TANK binding kinase 1 (TBK1), and the IkappaB kinase (IKK)ε [Fitzgerald et al. Nat Immunol 4, 491-496 (2003); Sharma et al. Science 300, 1148-1151 (2003)] that subsequently lead to activation of IRF-3 and other transcription factors. As the intracellular triggering of DC maturation by viruses requires virus replication [Lopez et al. supra (2003); Lopez et al. J Immunol 173, 6882-6889 (2004)], the present inventors evaluated whether an increased amount of dsRNA produced during viral replication might account for the potent induction of type I IFNs by SeV-C. To this end, the ability of the influenza NS1 protein to bind dsRNA thereby blocking the induction of type I IFN expression was used to advantage [Donelan et al. J Virol 77, 13257-13266 (2003)]. NS1 contains an N-terminal dsRNA binding domain between amino acids 1-73 and an effector domain at residues 74-237. Transfection of the NS1 dsRNA binding domain inhibited the induction of IFNβ by both SeV-52 and SeV-C. However, SeV-C required a higher amount of NS1 to reduce the IFNβ promoter induction to a level similar to that of SeV-52 (FIG. 2A). These data suggest that the disparity between SeV-52 and SeV-C may be explained by differences in the activating dsRNA produced by the viruses.

To evaluate if the rate of transcription and protein synthesis of SeV-C is a major determinant in its generation of highly activating replication intermediates, the expression of viral proteins was examined at various times after infection. SeV genomes contain six major proteins: NP, P, M, F, HN, and L expressed in a gradient with NP having the highest level of expression [Kolakofsly et al. Virology 318, 463-473 (2004)]. Cells were infected with equal MOIs of SeV-52 or SeV-C for 3, 6, or 24 h. Quantitative analysis of mRNA extracted from these cells revealed that, at all time points, the cells infected with SeV-52 produced more viral NP mRNA than cells infected with SeV-C (FIG. 2B). These results agree with the present inventors' previously reported expression of the HN and F proteins 24 h after infection with these viruses [Lopez et al. supra (2003)]. Since SeV-C produces viral NP mRNA and HN and F proteins more slowly than its counterpart SeV-52, neither an increase in viral mRNA nor viral protein production is responsible for the amplification of DC-activating stimulus in SeV-C infected cells. Therefore, the most probable source of the strong ability of SeV-C to induce DC maturation and type I IFN production is uniquely stimulatory dsRNA intermediates formed during SeV-C replication.

SeV-C stocks contain higher proportions of DI particles than SeV-52: Viral DI particles contain incomplete genomes that replicate only in the presence of standard virus. DI particles are enriched in co-infected cells as they replicate more efficiently than standard virus genomes due to their smaller length and differential promoter sensitivities [Calain & Roux. Virology 212, 163-173 (1995); Tapparel & Roux. Virology 225, 163-171 (1996)]. Thus, they interfere with standard virus protein production by monopolizing the viral replication machinery. Undiluted passages of SeV can result in enhanced DI particle production and their presence has been shown to increase the induction of type I IFNs [Poole et al. supra (2002); Johnston. supra (1981)]. Based upon the observations that SeV-C produces proteins less efficiently than SeV-52 and has potentiated type I IFN induction abilities, the present inventors hypothesized that SeV-C stocks may have high levels of DI particles that could be responsible for their superior DC maturing ability (FIGS. 1A, 2B, and Lopez et al. supra (2003)).

The presence of DI particles can be detected using a well characterized method of calculating the ratio between infectious viral particles and total viral particles (DI particles plus standard infectious virus) [Johnston. supra (1981)]. SeV-52 and SeV-C stocks were analyzed for their total particle titers using a hemagglutination (HA) assay based on the binding of chicken red blood cells (RBCs) to the external HN protein of SeV particles. Infectious particle quantifications (TCID₅₀) were obtained by titration in LLCMK2 cells. As shown in Table 1, SeV-52 and SeV-C stocks have equivalent TCID₅₀ values. However, the SeV-C stock contained twice as many hemagglutinating particles as SeV-52. This lower infectivity to total HA particle (I/HA) ratio of SeV-C compared to SeV-52 (Table 1) suggests a higher content of DI particles in SeV-C.

TABLE 1
I/HA ratios indicate higher levels of DI particles in standard SeV-C stocks than in
standard SeV-52 stocks. High dilution passages of SeV-C increase its I/HA ratio,
indicating a lowered proportion of DI particles while SeV-52 can be grown to
enhance its DI particle content.

| | | | HA Titer | | | |
|---|---|---|---|---|---|---|
| | | | (Total | TCID₅₀/25 uL (Log10) | | Referred to |
| Virus | Passage | Dilution | Particles) | (Infectious Particles) | I/HA | as |
| 52 | Standard | 10³ | 512 | 6.9 | 1.5 × 10⁴ | |
| 52 | 2 | 0 | 8192 | 7.7 | 6.1 × 10³ | 52 hi DI |
| C | Standard | 10³ | 1024 | 6.9 | 7.8 × 10³ | |
| C | 1 | 10⁷ | 32 | 6.4 | 7.8 × 10⁴ | |
| C | 2 | 10⁶ | 128 | 6.4 | 2.0 × 10⁴ | C low DI |

DI particles are necessary for enhanced DC activation abilities of SeV-C: To test the hypothesis that DI particles are responsible for the strong DC maturation ability of SeV-C, the present inventors manipulated its DI particle content by modifying the virus growth conditions. To purify standard virus, SeV-C was inoculated into eggs at a dilution of 1:10⁷. At this dilution only 33% of infected eggs tested positive for virus at 40 hours post infection (hpi) indicating that the average viral inoculum was less than one infectious dose per egg. By pooling the positive eggs, a viral stock was created that was decreased in its DI particle content as indicated by its raised I/HA ratio compared to the original SeV-C stock (Table 1). This stock was then further passaged at a 1:10⁶ dilution creating the stock herein referred to as SeV-C low DI (Table 1).

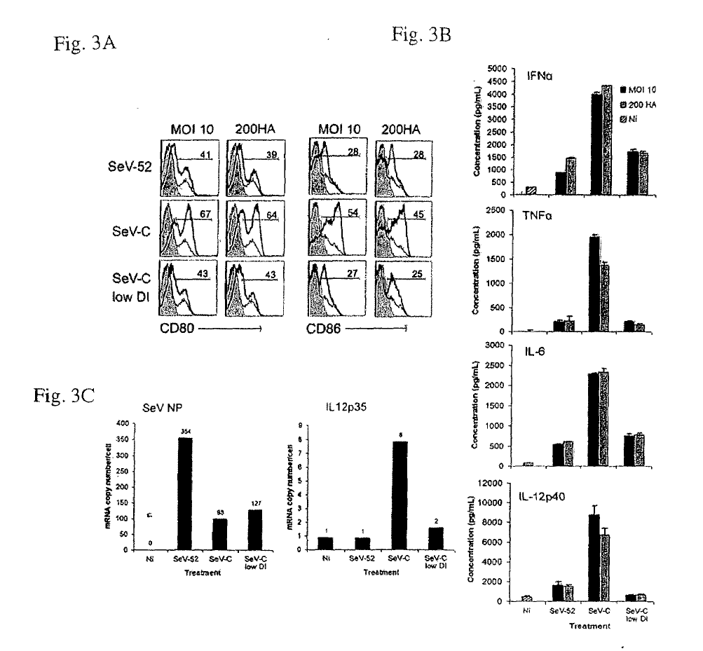

Virus preparations with different DI particle content were tested for their abilities to induce maturation of mouse BM-DCs. SeV-C low DI had a significantly reduced ability to mature DCs as measured by the up-regulation of costimulatory molecules (FIG. 3A), cytokine secretion (FIG. 3B), and IL-12p35 mRNA production (FIG. 3C). Measurement of viral NP mRNA in cells infected with equal MOIs indicated that all viruses tested were actively replicating (FIG. 3C) though, as seen previously, levels of viral mRNA production do not correlate with DC maturation ability. Additionally, equalizing HA titers (200 HA units) of the various virus preparations used to infect BM-DCs demonstrated that the total number of particles is not the factor responsible for the potent induction of DC maturation by SeV-C (FIG. 3A,B). Rather, the DC maturation ability of SeV-C is directly influenced by its DI particle to standard virus ratio. These results demonstrate that DI particles contribute a unique stimulus for DC maturation that is not mimicked by equilibrating the total number of viral particles.

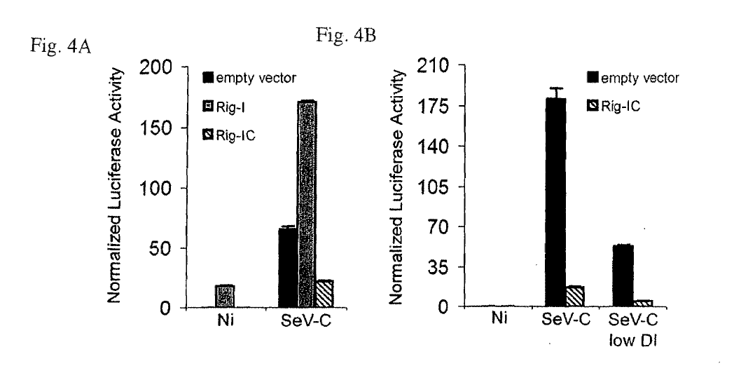

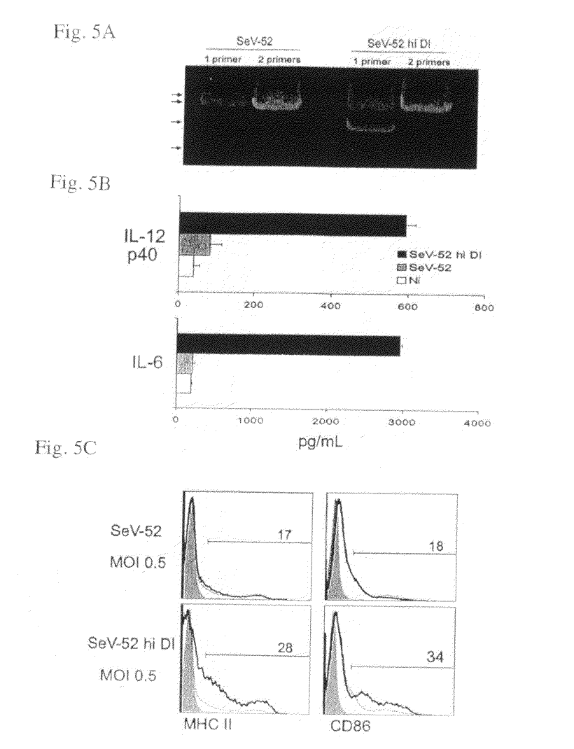

DI particle-mediated DC activation requires signaling through RIG-I: RIG-I is a dsRNA binding protein known to be involved in the innate immune response to virus infection [Yoneyama et al. supra 2004)]. Taking into consideration the potentially increased dsRNA production of viruses high in DI particle content as compared to standard virus (FIG. 2A), the present inventors tested RIG-I for its involvement in signaling leading to the enhanced activating abilities of viruses high in DI particle content. As shown in FIG. 4A, overexpression of RIG-I results in superior IFNβ promoter induction by SeV-C while the presence of RIG-IC [Yoneyama et al. supra (2004)], a dominant negative form of RIG-I, eliminates IFNβ promoter induction. Comparison of SeV-C and SeV-C low DI further illustrates the requirement of RIG-I signaling for the enhanced activation ability of the DI particle-rich viruses. Both SeV-C and SeV-C low DI lose their type I IFN induction ability in the presence of a RIG-I dominant negative protein (FIG. 4B). This suggests that the enhanced production of dsRNA by virus in the presence of DI particles results in a heightened activation of the RIG-I signaling pathway with the consequent increase in the expression of type I IFN.

Increasing DI particle content enhances DC maturation by a weakly stimulatory virus: As previously described, SeV-52 is both a weak inducer of type I IFN and of DC maturation. Two undiluted passages of this virus significantly decrease its I/HA ratio reflecting an increased proportion of DI particles in this preparation (Table 1). It has been reported that DI particles with genomes flanked by the viral antigenomic promoter are responsible for enhancing type I IFN induction in virus infection [Marcus & Gaccione. Virology 171, 630-633 (1989)]. A standard RT-PCR assay can be performed to specifically amplify this type of DI species using only one primer complementary to the antigenomic promoter of the virus. Using this assay, four distinct bands can be detected from amplified SeV-52 RNA (FIG. 5a). When an equivalent amount of SeV-52 hi DI RNA was reverse transcribed and amplified, bands representing DI particle species are increased in intensity with a single DI species predominating (FIG. 5A). Thus, confirming the increase in DI particle content indicated by its I/HA ratio, SeV-52 hi DI contains higher levels of antigenomic promoter-flanked DI species than SeV-52.

To test whether an increase in DI particle content affects the ability of SeV-52 to mature DCs, BM-DCs were infected with equal MOIs of SeV-52 and SeV-52 hi DI. Interestingly, SeV-52 hi DI increased the secretion of pro-inflammatory cytokines, IL-6 and IL-12, from infected mouse DCs relative to standard SeV-52 (FIG. 5B). Additionally, upregulation of CD86 and MHC II surface expression were significantly increased by SeV-52 hi DI as compared to cells infected with SeV-52 (FIG. 5C). Therefore, these data provide further evidence that a high DI particle content including antigenomic promoter-flanked DI species can be utilized to enhance the DC maturation ability of SeVs.

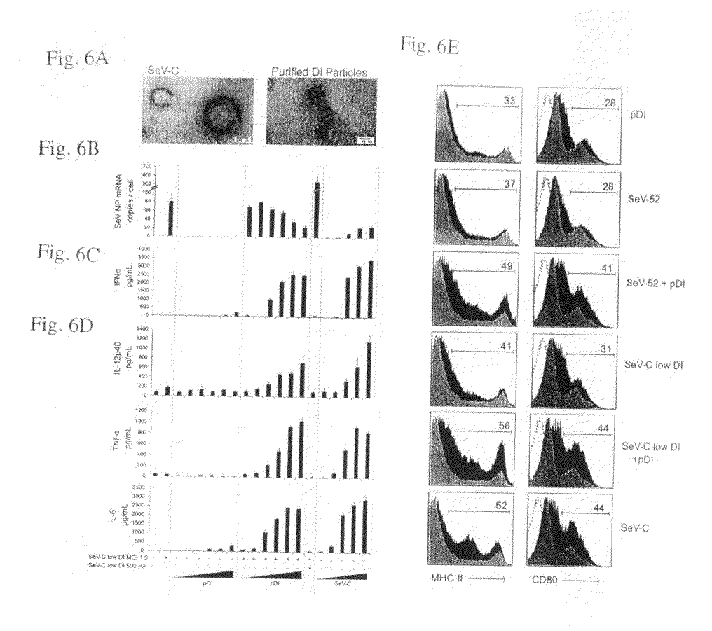

Purified DI particles enhance the DC maturation ability of SeVs: Purified (p) DI particles were isolated from SeV-C using sucrose gradients as previously described [Johnston. supra (1981)]. SeV-C and pDI particles were visualized by electron microscopy. SeV-C stocks were found to contain a polymorphic population of particles ranging in size from 50 to 250 nm in diameter, whereas pDI particles are at the smaller end of this size range with the majority having a diameter less than 100 nm (FIG. 6A). The pDI particle preparation had an I/HA ratio of 80 indicating a dramatic reduction in the proportion of infectious particles compared to SeV-C (Table I). The pDI particles were assessed by three criteria: 1) inability to replicate in the absence of standard virus, 2) interference with viral protein production, and 3) enhancement of type I IFN induction by standard virus. BM-DCs were treated with increasing doses of pDI particles from 5 to 500 HA units and even at the highest dose tested, no significant viral replication was detected as measured by qRT-PCR of NP mRNA transcripts (FIG. 6B). In contrast, infection with increasing doses of SeV-C, the parental virus, shows an increase in NP mRNA correlating with the dose. Likewise, infection with SeV-C low DI results in robust transcription of NP mRNA. Conversely, NP mRNA production by SeV-C low DI is inhibited when cells are co-infected with pDI particles. As expected, type I IFN was not induced by the pDI particles alone, but co-infection with standard replicative virus allowed high induction of IFNα similar to levels induced by SeV-C (FIG. 6C). Thus, the pDI particles conform to the criteria for DI particles.

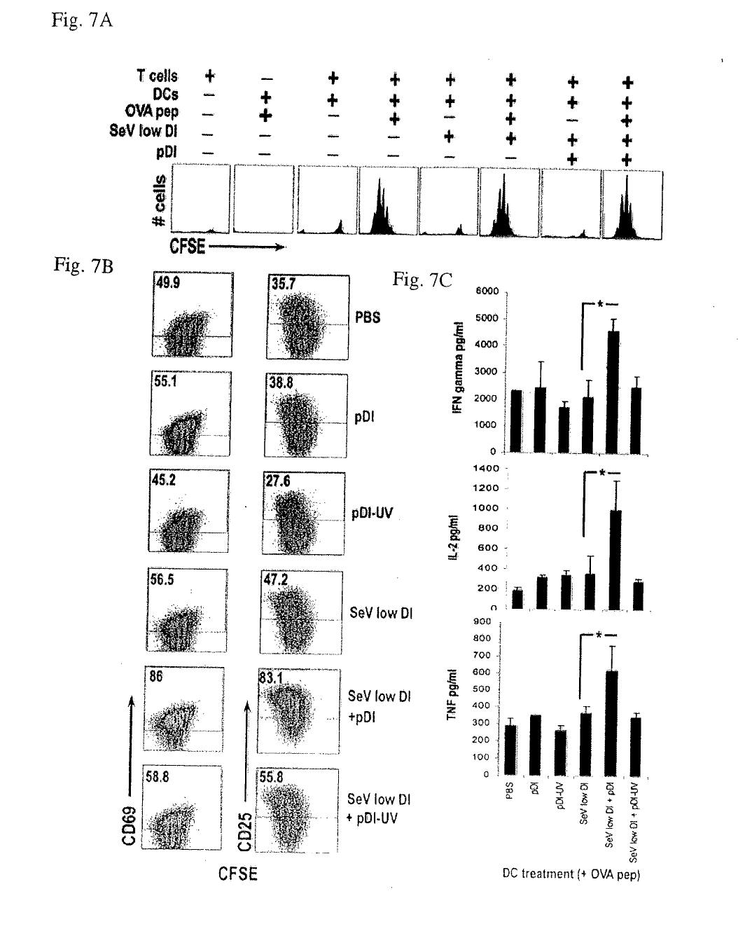

Confirming the effect of DI particles on DC maturation, pDI particles enhanced the secretion of the pro-inflammatory cytokines IL-6, TNFα and IL-12 p40 (FIG. 6D), as well as surface expression of CD80 and MHC II (FIG. 6E), in BM-DCs co-infected with standard virus. Similar results were obtained using SeV-52 as the standard virus (FIG. 6E), demonstrating that a virus that weakly stimulates DCs can gain potent DC maturation ability with the addition of pDI particles. Furthermore, DCs matured by SeV in the presence of pDI particles are better able to activate naive TCR transgenic OT-I cells (FIG. 7). An enhanced expression of the T cell activation markers CD69 and CD25 (FIG. 7B), as well as secretion of IFNγ, IL-2 and TNFαα(FIG. 7C), was observed when OT-I cells were primed by DCs activated in the presence of pDI particles. The activation ability of pDI particles was hindered by their UV inactivation (FIGS. 7B and C) showing that the generation of DI particle replication intermediaries is essential for their activity. FIG. 7A shows a control experiment demonstrating that DCs presenting the OVA SIINFEKL peptide (SEQ ID NO: 18) specifically stimulated T cell proliferation. These experiments demonstrated that DCs matured in the presence of DI particles show an enhanced ability to prime naive CD8⁺ T cells.

Discussion

Prior studies by the present inventors demonstrated that the strong type I IFN induction ability of SeV-C correlates with its potent ability to induce DC maturation Lopez et al. supra (2003)]. In order to gain further insight into the mechanism responsible for the intracellular triggering of type I IFN and DC maturation by viruses, the features of SeV-C that endow it with these unique properties were delineated. In the present study, comparisons of SeV-C and SeV-52 demonstrate that the former induces IFN, more quickly and potently than SeV-52 (FIG. 1A). Notably, SeV-C does not seem to lack an IFN induction antagonist (FIG. 2A and B) and it maintains its ability to block STAT1 phosphorylation (FIG. 1F). Instead, SeV-C provides more activation stimuli or molecular patterns capable of triggering IFNβ promoter induction than SeV-52 (FIG. 2D).

By calculating I/HA ratios for SeV-52 and SeV-C grown at different dilutions and time points, the present inventors show that SeV-C contains more naturally occurring DI particles than SeV-52 (FIG. 3). Although the passage histories of SeV-52 and SeV-C have been identical for many years in the laboratory of the present inventors, the original passage histories of these viruses are not documented. Because SeV-C may have been successively passaged without dilution, DI particles may have been propagated from earlier passages. Alternatively, the present studies do not exclude the possibility that SeV-C possesses a genetic predisposition to produce higher levels of DI particles, although results presented herein demonstrate that it is possible to generate SeV-C stocks with much lower levels of these particles.

SeVs are known to produce several species of DI particles. Internal deletion DI particles are missing internal sequences, making them replication incompetent even though they contain both genomic and antigenomic promoters allowing transcription and replication. Copy-back DI particles lack the genomic promoter which is replaced by the antigenomic promoter preventing transcription but allowing replication. It has been shown that copy-back DI particles replicate up to 20 times faster than species having both genomic and antigenomic promoters due to specific sequences within the antigenomic promoter that confer higher replication abilities [Calain & Roux. supra (1995); Tapparel & Roux. supra (1996); Re & Kingsbury. J Virol 58, 578-82 (1986)). The present studies reveal that SeVs naturally produce antigenomic promoter-flanked DI particles, most probably of the copy-back type, and that viruses with enhanced DC activation abilities have increased levels of these DI particles. The faster replication of DI genomes provides a mechanism by which they act as enhancers of virus-associated molecular patterns by supplying higher levels of replication intermediaries that trigger type I IFN and DC maturation pathways.

These findings confirm the present inventors' previously reported correlation between type I IFN induction and DC maturation, even in the absence of secreted type I IFN signaling [Lopez et al. supra (2003)] and TLR signaling [Lopez et al. supra (2004)], providing evidence that the type I IFN and DC maturation pathways induced by intracellular viral replication share common molecules. The protein RIG-I has been shown to interact directly with dsRNA leading to induction of type I IFN [Yoneyama et al. supra (2004)] and is a likely candidate molecule for involvement in DC maturation in response to virus since other known dsRNA binding proteins, TLR3 and PKR have been shown to be dispensible for DC maturation [Honda et al. supra (2003); Lopez et al. supra (2004); Lopez et al. supra (2003); Barchet et al. Eur J Immunol 35, 23642 (2005)]. Indeed, RIG-I is necessary for efficient type I IFN induction by DI particle-rich viruses (FIG. 6). These results, in combination with previous evidence that type I IFN induction and DC maturation pathways share common molecules and that RIG-I has been shown to be involved in viral activation of NF-κB and induction of the cytokines IL-12 and IL-6 [Foy et al. Proc Natl Acad Sci USA 102, 2986-91 (2005)], strongly suggest that RIG-I plays a role in pathways leading to virus-induced DC maturation.

The present demonstration that addition of DI particles to a weak activator of DCs dramatically enhances its immunogenicity and ability to promote DC maturation represents a considerable advance in vaccine development. Indeed, SeVs have recently been shown to be promising vaccine virus candidates as they are generally well tolerated in humans and provide cross reactive immunity against human parainfluenza virus type I [Slobod et al. Vaccine 22, 3182-3186 (2004)]. Addition of DI particles to these live-virus vaccine preparations would enhance their immunogenic potential. Vesicular stomatitis virus has also been shown to acquire the ability to induce type I IFN specifically in the presence of copy-back DI particles, suggesting that this mechanism is applicable to other pathogenic viruses [Marcus & Gaccione. Virology 171, 630-3 (1989)]. Similar effects remain to be studied in other paramyxoviruses of clinical relevance such as measles and RSV, which also naturally produce DI particles [Whistler et al. Virology 220, 480-4 (1996); Valdovinos & Gomez. Intervirology 46, 190-8 (2003)]. DI particles enhance the viral stimulus for DC activation, while decreasing viral protein synthesis and thus replication, and thereby enhance the safety of a live virus vaccine.

DI particle enhancement of viral molecular patterns also has the advantage of maturing only infected DCs. This stands in contrast to existing adjuvants, such as CpG oligonucleotides, which nonspecifically activate DCs. Another advantageous feature of using DI particles as adjuvants is that DI particles are natural, biodegradable adjuvants. Thus, knowledge of DI particle enhancement of virus immunogenicity has a dramatic impact on the rational design of viral vaccines. Furthermore, the robust immune response generated by DI particle-rich SeV, which is well tolerated in humans, suggests its possible use as an adjuvant for other types of immunization.

### Example II

The present inventors have also demonstrated that a DI-rich virus preparation may be used to advantage as an adjuvant for enhancing immune responses to a specific antigen in vivo. As shown herein, ovalbumin (OVA) was used as an exemplary antigen against which an immune response was elicited in an animal model system.

Methods and Materials

C57BL/6 mice were immunized in the footpad with 20 ug ovalbumin (OVA) mixed with either PBS, Standard SeV (strain 52, 2000 TCID50) or hi DI-SeV (200 TCID50). Two weeks after infection, splenocytes from naive mice were pulsed at 4×10⁷ cells/ml with 20 μM OVA peptide, 20 μM SeV NP₃₂₄₋₃₃₂ peptide, or PBS (without peptide) for 15 minutes at room temperature. The cells were subsequently labeled at 2×10⁷ cells/ml with different concentrations of CFSE (Molecular Probes, Eugene, Oreg.) (0.05, 1 or 5 μM respectively). The cells were then washed, mixed, and injected intravenously (iv) into the immunized mice. Twenty hours (20 h) after the injections, spleens were harvested and single cell suspensions were prepared and analyzed by flow cytometry.

As shown in the histograms depicted in FIGS. 11A-E, the disappearance of a peak indicates that CTLs against that specific peptide have been generated in the immunized animals and those CTLs lysed the CFSE-labeled target cells. This experiment shows that the presence of hi-DI virus (i.e., a DI-enriched population) acts as an effective adjuvant for enhancing immune responses to an antigen (e.g., OVA), as evidenced by the induction of anti-OVA CTLs. The response to SeV was used as a negative control for the purposes of this experiment.

### Example III

To explore further the effect of DI particles on DC maturation, the present inventors performed experiments using human monocyte-derived DCs. Briefly, human DCs were grown from CD14+ monocytes in the presence of GMCSF. The cells were infected with SeV at an MOI=0.5, treated with 100 ng LPS, or mock infected with PBS (NI) and cultured for 24 h. As shown in FIG. 14A, the presence of SeV-C (hi DI particle population) induced human DCs to produce higher levels of cytokines (IL-12, L-10, and TNFα) relative to those levels observed for DCs infected with SeV-52. Cytokines in the supernatants were measured by capture ELISA. As shown in FIG. 14B, expression of the SeV F and HN proteins was also assessed. Consistent with results presented in Example I, viral protein levels in SeV-52 infected DCs were significantly higher than those of SeV-C infected DCs. The level of costimulatory molecule CD86 in the DC surface was also measured by flow cytometry 24 h post-infection. As shown in FIG. 14C, CD86 expression is highest in DCs infected with SeV-C and LPS treated DCs. CD86 expression was demonstrably lower in DCs infected with SeV-52. LPS was used as a positive control in these experiments as indicated.

In summary, therefore, human monocyte-derived DCs mature more efficiently upon infection with SeV with a high content of DI particles (SeV-C) than with a virus containing a low content of DI particle (SeV-52). Moreover, the data also show that DI particles have the added benefit of inhibiting viral protein production while enhancing DC maturation.

### Example IV

The effect of DI particle-induced maturation of DCs was further investigated to determine if T cells stimulated with such matured DCs were activated in an enhanced manner in the absence of type I IFN and if the ratio of DC to T cell played a role in the enhancement. In brief, mouse bone marrow-derived DCs were treated with PBS, SeV low DI (CB), CB plus 100 HA purified DI particles (DI), LPS, or left untreated (alone), in the presence of 10 μg/ml of OVA₂₅₇₋₂₆₄ peptide for 1 h. Treated DCs were cultured in a 1:10 or 1:80 ratio with CSFE-labeled CD8 T cells transgenic for the OVA₂₅₇₋₂₆₄ peptide T cell receptor. After two days in culture the cells were analyzed for proliferation and the supernatant analyzed for the presence of IFN gamma by capture ELISA.

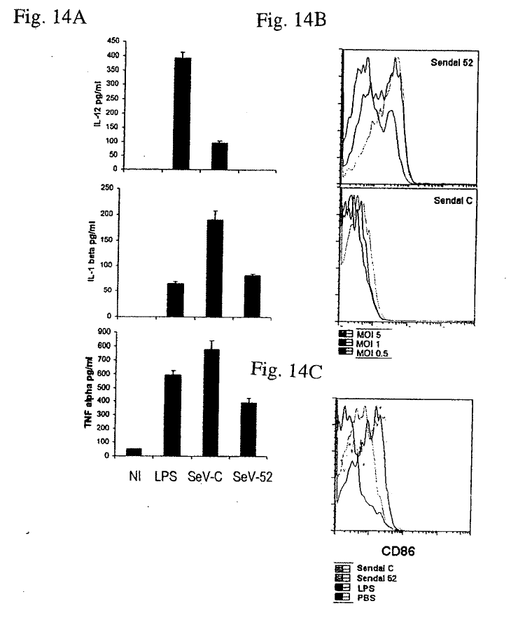

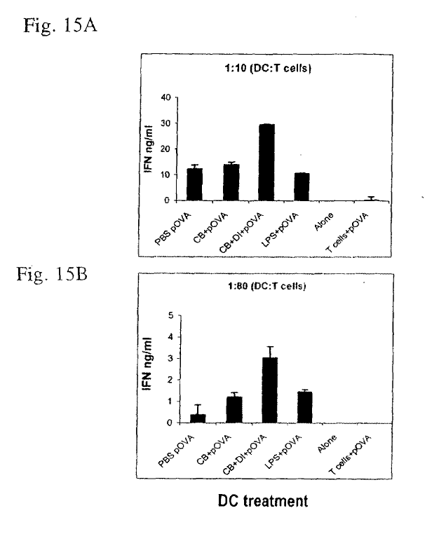

As shown in FIGS. 15A and B, DCs infected with SeV in the presence of DI particles induce more efficient secretion of IFN gamma from T cells than DCs infected in the absence of DI particles. Moreover, the ratio of DC to T cell impacts the degree of enhancement. These data corroborate those of FIG. 7 and demonstrate that the enhanced maturation of DCs provided by DI particles correlates with an improvement in their ability to stimulate CD8 T cells.

### Example V

The effect of supplementing a low DI SeV population (e.g., SeV-52 or SeV-Cb) with purified DI particles was also investigated. In short, mouse bone marrow-derived DCs were grown from type I IFN receptor knock out (KO) mice or wild type (wt) Sv129 controls and treated with PBS (NI), purified DI particles (pDI), SeV-52, or SeV-C low DI (Cb), or combinations thereof, as indicated. SeV-C was used as a control. Cytokines in the supernatants were measured by capture ELISA. Determination of IL-6 and TNF gave similar results.

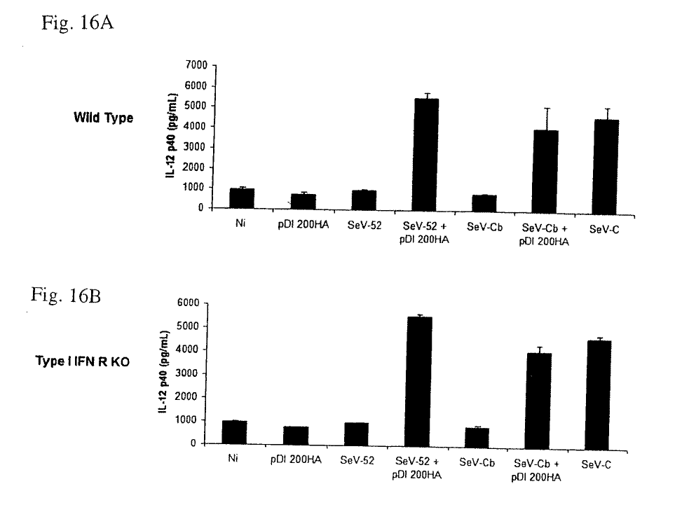

As shown in FIGS. 16A and B, DI particles purified from SeV-C enhance the DC maturation ability of other SeV strains. As such, these results demonstrate the utility of a heterologous DI particle-enriched viral population. These results also reveal that the mechanism for the enhancement of DC maturation afforded by DI particles is independent of secreted type I IFN.

### Example VI

The contribution of virus and DI particle replication to the process of DC maturation were next evaluated. Briefly, mouse bone marrow-derived DCs were treated as indicated with PBS (NI), purified DI particles (pDI), SeV-52, SeV-C low DI (CB) or UV-inactivated viruses or pDI particles. Cytokines in the supernatants were measured by capture ELISA 24 h after treatment. Quantification of viral NP protein expression from mRNA extracted from infected cells was performed by qPCR.

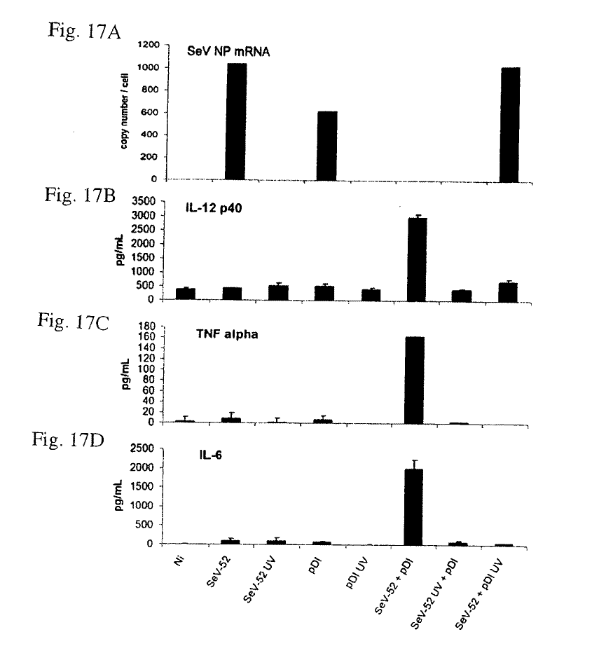

As shown in FIGS. 17A-D, DI particles purified from SeV-C enhance DC maturation through a mechanism dependent on the generation of virus replication intermediaries. These findings are consistent with those presented in Example I, FIG. 6.

### Example VII

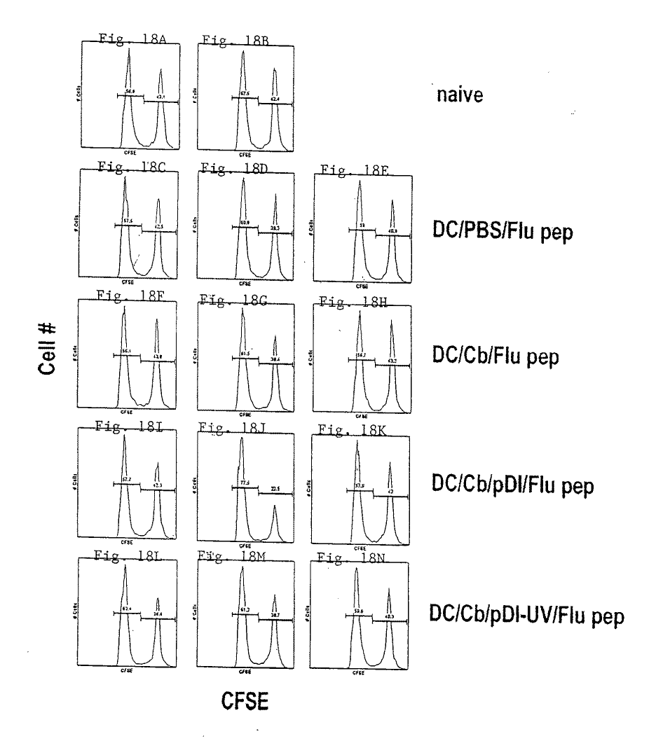

To investigate further the ability of DCs activated ex vivo to augment immune responses upon introduction into a subject, the present inventors performed the following experiment. C57BL/6 mice were immunized with bone marrow-derived DCs treated as indicated: Cb, SeV-C low DI at a multiplicity of infection (moi) of 2; pDI, purified DIs 100 HA units; Flu pep, ASENENMETM (SEQ ID NO: 58); and/or pDI-UV, UV inactivated pDIs. Seven days post-infection, 3 animals per group were injected with CFSE-labeled splenocytes pulsed with the Flu peptide (high CFSE dose, second pick) or non pulsed (low CFSE dose, first pick). Twenty-four hours later, animals were sacrificed and splenocytes analyzed for the presence of labeled cells by flow cytometry. Reduction in the pick size indicates labeled splenocyte cell death, which is an indicator of an enhanced immune response. As shown in FIG. 18J, DCs activated ex vivo with Cb, pDI, and Flu peptide triggered a T cell mediated immune response in one of the treated mice as evidenced by a reduced pick size (right peak in flow cytometry histogram) corresponding to the number of labeled, intact splenocytes. Similar results were not observed in any of the animals treated with PBS and Flu peptide (FIG. 18C-E); Cb and Flu peptide (FIG. 18F-H); or Cb, pDI-UV, and Flu peptide (FIG. 18L-N). The results, in particular those noted for animals treated with Cb and Flu peptide (FIG. 18F-H); and Cb, pDI-UV, and Flu peptide (FIG. 18L-N) reveal that the presence of functional DI particles during ex vivo activation of DCs augments their activation levels as reflected in their ability to trigger effector cells in vivo.

These results corroborate the evidence presented in the above examples by demonstrating that DCs activated with viruses in the presence of DI particles are more efficient in priming cytotoxic T cells than viruses lacking DI particles. This DI particle-mediated effect is dependent on their replication capacity, since UV inactivation renders them essentially ineffectual with respect to inducing an enhanced immune response to antigen.

This experiment evaluated the generation of primary effector cytotoxic T cells (CTLs). These cells are generated when a previously non-immune animal is exposed to the antigen for the first time, and are responsible for the clearance of infected cells. This is, therefore, not a memory response. The primary CTL population constricts in the absence of stimuli and is eventually so reduced in number as to be functionally non-existent. The experiment performed used DCs bearing a Flu peptide as antigen. Since this is a non-replicative stimuli, the primary CTL response may have been evaluated too late (day 8) to detect primary killing. To address this issue, the experiment will be performed at an earlier time point to ensure detection of CTL cells. Indeed, the standard protocol for evaluation of primary CTLS is at day 6 post-infection.

The number of DCs could also affect the outcome of immunization. Higher numbers and even repeated injections may be used to improve the immunization procedure.

Other parameters of the immune response may also be optimized, such as protection from challenge with a Flu virus or the generation of memory CTL precursors. These methods may prove more sensitive than the direct evaluation of primary CTLs.

Multiple peptides representing a boarder range of epitopes may also be used to improve the efficiency of the immunization.

While certain of the particular embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for stimulating an immune response in a subject, comprising:
administering to a subject at least one antigen, wherein said at least one antigen is administered in conjunction with a defective interfering (DI) particle-enriched viral population and said at least one antigen and said DI particle-enriched viral population are administered in an effective amount capable of inducing an antigen specific immune response in said subject.

2. The method of claim 1, wherein said at least one antigen comprises a peptide, a polypeptide, a cell, a cell extract, a polysaccharide, a polysaccharide conjugate, a lipid, a glycolipid, a carbohydrate, a virus, a virus vaccine, a viral extract or a polypeptide encoded by a nucleic acid.

3. The method of claim 1, wherein said at least one antigen is a tumor cell antigen, or an allergen, or is isolatable from an infectious agent, wherein said infectious agent is a virus, bacterium, fungus, or parasite.

4. The method of claim 1, wherein the subject is infected with a virus, bacteria, fungus or parasite.

5. The method of claim 1, wherein the subject is afflicted with a neoplastic disorder.

6. The method of claim 1, wherein the subject is a vertebrate.

7. The method of claim 1, wherein said at least one antigen is encoded by a recombinant standard virus or a recombinant DI particle present in said DI particle-enriched viral population.

8. The method of claim 1, wherein said DI particle-enriched viral population is a homologous DI particle-enriched viral population or a heterologous DI particle-enriched viral population, wherein said DI particle-enriched viral population comprises standard virus capable of complementing DI particles in said DI particle-enriched viral population.

9. A method for stimulating an immune response in a subject, comprising:
administering to a subject a homologous or heterologous DI particle-enriched viral population, wherein said DI particle-enriched viral population comprises a recombinant standard virus encoding at least one antigen and DI particles, and wherein said recombinant standard virus is capable of complementing said DI particles and said homologous or heterologous DI particle-enriched viral population is administered in an effective amount capable of inducing an antigen specific immune response in said subject.

10. The method of claim 9, wherein recombinant standard virus encoding at least one antigen is a recombinant paramyxovirus or a recombinant orthomyxovirus.

11. The method of claim 9, wherein said at least one antigen is a peptide or a polypeptide.

12. The method of claim 9, wherein said at least one antigen is a tumor cell antigen, or an allergen, or is isolatable from an infectious agent, wherein said infectious agent is a virus, bacterium, fungus, or parasite.

13. The method of claim 9, wherein the subject is infected with a virus, bacteria, fungus or parasite.

14. The method of claim 9, wherein the subject is afflicted with a neoplastic disorder.

15. The method of claim 9, wherein the subject is a vertebrate.

16. A method for activating a dendritic cell, comprising:
contacting a dendritic cell with at least one antigen, wherein said at least one antigen is administered in conjunction with a DI particle-enriched viral population, in an effective amount to activate a dendritic cell.

17. The method of claim 16, wherein the dendritic cell is activated ex vivo.

18. The method of claim 17, further comprising administering the activated dendritic cell to a subject to promote an immune response to the at least one antigen.

19. The method of claim 16, wherein the at least one antigen is encoded by a recombinant standard virus or recombinant DI particle of said DI particle-enriched viral population.

20. The method of claim 16, wherein said antigen is a tumor cell antigen, or an allergen, or is isolatable from an infectious agent.

21. A method for activating a dendritic cell, comprising:
contacting a dendritic cell with at least one antigen, wherein said at least one antigen is administered in conjunction with a plurality of recombinant packaging defective DI particles, in an effective amount to activate a dendritic cell.

22. The method of claim 21, wherein the dendritic cell is isolated from a subject and activated ex vivo.

23. The method of claim 22, further comprising administering at least one activated dendritic cell to a subject in an effective amount to promote an immune response to the at least one antigen.

24. The method of claim 21, wherein the at least one antigen is encoded by the recombinant packaging defective DI particles.

25. The method of claim 21, wherein said antigen is a tumor cell antigen, or an allergen, or is isolatable from an infectious agent.

26. A method for activating a dendritic cell, comprising:
transfecting a dendritic cell with a plurality of recombinant packaging defective DI constructs, wherein the recombinant packaging defective DI constructs comprise a nucleic acid sequence encoding at least one antigen, in an effective amount to activate a dendritic cell.

27. The method of claim 26, wherein the dendritic cell is isolated from a subject and activated ex vivo.

28. The method of claim 27, further comprising administering an activated dendritic cell to a subject in an effective amount to promote an immune response to the at least one antigen.

29. The method of claim 26, wherein said antigen is a tumor cell antigen, or an allergen, or is isolatable from an infectious agent.

30. A mixture of a DI particle-enriched viral population and a conventional vaccine, wherein the conventional vaccine is a subunit vaccine, a recombinant live viral-delivery vector, a bacterial vaccine-delivery vector, a nucleic acid vaccine, virus-like particles (VLPs), a modified virus vaccine, an inactivated virus vaccine, or a live attenuated virus vaccine.

31. A composition comprising the mixture of claim 30 and a pharmaceutically acceptable carrier.

32. A heterologous DI particle-enriched viral population.

33. The heterologous DI particle-enriched viral population of claim 32, wherein said heterologous DI particle-enriched viral population comprises standard virus of a first paramyxovirus strain and DI particles of a second complementary paramyxovirus strain.

34. A composition comprising the heterologous DI particle-enriched viral population of claim 32 and a pharmaceutically acceptable carrier.

35. A recombinant packaging defective paramyxovirus or orthomyxovirus comprising a complementary antigenomic promoter.

36. A recombinant packaging defective paramyxovirus or orthomyxovirus of claim 35, further comprising a nucleic acid sequence encoding an antigen.

37. A composition comprising the recombinant packaging defective paramyxovirus or orthomyxovirus of claim 35 and a pharmaceutically acceptable carrier.

38. A method for stimulating an immune response in a subject, comprising:
administering to a subject a packaging defective recombinant virus, wherein said packaging defective recombinant virus is derived from a virus capable of generating DI particles, wherein said packaging defective recombinant virus comprises a complementary antigenomic promoter and a nucleic acid sequence encoding at least one antigen, and said packaging defective recombinant virus is administered in an amount capable of inducing an antigen specific immune response in said subject.
